SEQUENCE LISTING (73 sequences)

SEQ ID NO: 1 - DNA, 990 bp, Synthetic construct
  source 1..990: mol_type = other DNA
cacggtaatgttatggtgggtttcttgctttcagcctcgcttttataaacttcccctaacgttttactgctcgaatttactccacatgcactactgtatgactcataaaacatgctaatcattgtgaatatcgggacagcttaagagcaagaggaaatgcgccctctgcgcactgacggagctgggttaggtatgggagctccgaggctgtcagtccaccaagggaagcttttgtccttccccttgagctgaagtcgcttcctgcaggccgtggaatagaagcctggcccccctcactctgcatcagctccttgttcctcaccttcaccttcctgtcgtctgcggtgatttcatcgaattccacgcccagtttgaaggaaatctcggtgtttttaaaagtactctctgaccggatggtgaccaaatccccatttacgctgatgatcatgttgggcttggccatgcctgccactttccttgtggcaaagcccactcctacagggagaaagagaacgcatgtaaagctaacatttgtgtgtgtgatgaagaagacaccagggtgccggaggctcctttgatgctctcagaacctgtcatccatccagatttactgtgcctgtctgctaactacactccttcagataagtccttaatttcaacaagaatgtattaagagattaaatcttcatatctcgatgtctatgtgttttatataggtctgtatgtctatatttattataatatgctaataataagtgtatgtatattatctctattttctatttgtgattgaggcagtttgaccattttattctttcttaattactaaaagttttttttttttttaaaatctaagtcaaaggccaggcctcacttagagttattgtgttaaaaggtcatctatttttgagtgaacaaaaatcacatttcacaacacttgttgccaaatttaattttctaggcttctttcccatattactccctcatgaa

SEQ ID NO: 2 - DNA, 1260 bp, Synthetic construct
  source 1..1260: mol_type = other DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 3 - DNA, 30 bp, Synthetic construct
  source 1..30: mol_type = other DNA
cacggtaatgttatggtgggtttcttgctt

SEQ ID NO: 4 - DNA, 33 bp, Synthetic construct
  source 1..33: mol_type = other DNA
ttcatgagggagtaatatgggaaagaagcctag

SEQ ID NO: 5 - DNA, 20 bp, Synthetic construct
  source 1..20: mol_type = other DNA
ttgtccttccccttgagctg

SEQ ID NO: 6 - DNA, 20 bp, Synthetic construct
  source 1..20: mol_type = other DNA
caggaaggtgaaggtgagga

SEQ ID NO: 7 - DNA, 18 bp, Synthetic construct
  source 1..18: mol_type = other DNA
gtgctaacaaagacggca

SEQ ID NO: 8 - DNA, 18 bp, Synthetic construct
  source 1..18: mol_type = other DNA
gcgtagaagccttttggc

SEQ ID NO: 9 - DNA, 35 bp, Synthetic construct
  source 1..35: mol_type = other DNA
ccggcttgtccttccccttgagctgaagtcgccgg

SEQ ID NO: 10 - DNA, 16 bp, Synthetic construct
  source 1..16: mol_type = other DNA
cccttgagctgaagtc

SEQ ID NO: 11 - DNA, 40 bp, Synthetic construct
  source 1..40: mol_type = other DNA
gggtttttttttttttttcctttttttttttccttttttt

SEQ ID NO: 12 - DNA, 42 bp, Synthetic construct
  source 1..42: mol_type = other DNA
gggtttttccctttttttcctttttttttttccttttttttt

SEQ ID NO: 13 - DNA, 40 bp, Synthetic construct
  source 1..40: mol_type = other DNA
gggtttttccctttttttccttttcctttttccttttttt

SEQ ID NO: 14 - DNA, 40 bp, Synthetic construct
  source 1..40: mol_type = other DNA
gggtttttccctttctttccttttcctttttccttttttt

SEQ ID NO: 15 - DNA, 40 bp, Synthetic construct
  source 1..40: mol_type = other DNA
gggtttttccctttctttccttttcctttttccttccttt

SEQ ID NO: 16 - DNA, 40 bp, Synthetic construct
  source 1..40: mol_type = other DNA
gggttttttcttttttttcctttttttttttccttttttt

SEQ ID NO: 17 - DNA, 40 bp, Synthetic construct
  source 1..40: mol_type = other DNA
gggttcttccctttctttccttttcctttttccttccttt

SEQ ID NO: 18 - DNA, 40 bp, Synthetic construct
  source 1..40: mol_type = other DNA
gagtttttttttcttttttcttttttttcttctttttttt

SEQ ID NO: 19 - DNA, 36 bp, Synthetic construct
  source 1..36: mol_type = other DNA
gagtttttcttttttcttttttttcttctttttttt

SEQ ID NO: 20 - DNA, 40 bp, Synthetic construct
  source 1..40: mol_type = other DNA
gagtttttcttttatttaaaaattttcttattttaaaatt

SEQ ID NO: 21 - DNA, 41 bp, Synthetic construct
  source 1..41: mol_type = other DNA
ccccctttttttttttttttttttttttttttttttttttt

SEQ ID NO: 22 - DNA, 41 bp, Synthetic construct
  source 1..41: mol_type = other DNA
ccccctttttttttttttttttccctttttttttttttttt

SEQ ID NO: 23 - DNA, 41 bp, Synthetic construct
  source 1..41: mol_type = other DNA
cccccttttttcctttttttttccctttttttttttttttt

SEQ ID NO: 24 - DNA, 41 bp, Synthetic construct
  source 1..41: mol_type = other DNA
cccccttttttccttcttctttccctttttttttttttttt

SEQ ID NO: 25 - DNA, 41 bp, Synthetic construct
  source 1..41: mol_type = other DNA
cccccttctttccttcttctttccctttttttttttctttt

SEQ ID NO: 26 - DNA, 41 bp, Synthetic construct
  source 1..41: mol_type = other DNA
cccccttttttttttttttcttttttttttttctttttttt

SEQ ID NO: 27 - DNA, 41 bp, Synthetic construct
  source 1..41: mol_type = other DNA
cccccttctttccttcttctttccctttttttttttctggt

SEQ ID NO: 28 - DNA, 41 bp, Synthetic construct
  source 1..41: mol_type = other DNA
tcacctttttttttttttttttttttttttttttttttttc

SEQ ID NO: 29 - DNA, 36 bp, Synthetic construct
  source 1..36: mol_type = other DNA
tcaccttttttttttttttttttttttttttttttc

SEQ ID NO: 30 - DNA, 40 bp, Synthetic construct
  source 1..40: mol_type = other DNA
ttcaccttttttttttatatattttttttttttttttttc

SEQ ID NO: 31 - DNA, 60 bp, Synthetic construct
  source 1..60: mol_type = other DNA
gggtttttttttttttttcctttttttttttcctttttttttgtccttccccttgagctg

SEQ ID NO: 32 - DNA, 60 bp, Synthetic construct
  source 1..60: mol_type = other DNA
gggtttttccctttttttcctttttttttttcctttttttttgtccttccccttgagctg

SEQ ID NO: 33 - DNA, 60 bp, Synthetic construct
  source 1..60: mol_type = other DNA
gggtttttccctttttttccttttcctttttcctttttttttgtccttccccttgagctg

SEQ ID NO: 34 - DNA, 60 bp, Synthetic construct
  source 1..60: mol_type = other DNA
gggtttttccctttctttccttttcctttttcctttttttttgtccttccccttgagctg

SEQ ID NO: 35 - DNA, 60 bp, Synthetic construct
  source 1..60: mol_type = other DNA
gggtttttccctttctttccttttcctttttccttcctttttgtccttccccttgagctg

SEQ ID NO: 36 - DNA, 60 bp, Synthetic construct
  source 1..60: mol_type = other DNA
gggttttttcttttttttcctttttttttttcctttttttttgtccttccccttgagctg

SEQ ID NO: 37 - DNA, 60 bp, Synthetic construct
  source 1..60: mol_type = other DNA
gggttcttccctttctttccttttcctttttccttcctttttgtccttccccttgagctg

SEQ ID NO: 38 - DNA, 60 bp, Synthetic construct
  source 1..60: mol_type = other DNA
gagtttttttttcttttttcttttttttcttcttttttttttgtccttccccttgagctg

SEQ ID NO: 39 - DNA, 56 bp, Synthetic construct
  source 1..56: mol_type = other DNA
gagtttttcttttttcttttttttcttcttttttttttgtccttccccttgagctg

SEQ ID NO: 40 - DNA, 60 bp, Synthetic construct
  source 1..60: mol_type = other DNA
gagtttttcttttatttaaaaattttcttattttaaaattttgtccttccccttgagctg

SEQ ID NO: 41 - DNA, 58 bp, Synthetic construct
  source 1..58: mol_type = other DNA
gggtttttttttttttttcctttttttttttcctttttttgtgctaacaaagacggca

SEQ ID NO: 42 - DNA, 60 bp, Synthetic construct
  source 1..60: mol_type = other DNA
gggtttttccctttttttcctttttttttttcctttttttttgtgctaacaaagacggca

SEQ ID NO: 43 - DNA, 58 bp, Synthetic construct
  source 1..58: mol_type = other DNA
gggtttttccctttttttccttttcctttttcctttttttgtgctaacaaagacggca

SEQ ID NO: 44 - DNA, 58 bp, Synthetic construct
  source 1..58: mol_type = other DNA
gggtttttccctttctttccttttcctttttcctttttttgtgctaacaaagacggca

SEQ ID NO: 45 - DNA, 58 bp, Synthetic construct
  source 1..58: mol_type = other DNA
gggtttttccctttctttccttttcctttttccttcctttgtgctaacaaagacggca

SEQ ID NO: 46 - DNA, 58 bp, Synthetic construct
  source 1..58: mol_type = other DNA
gggttttttcttttttttcctttttttttttcctttttttgtgctaacaaagacggca

SEQ ID NO: 47 - DNA, 58 bp, Synthetic construct
  source 1..58: mol_type = other DNA
gggttcttccctttctttccttttcctttttccttcctttgtgctaacaaagacggca

SEQ ID NO: 48 - DNA, 58 bp, Synthetic construct
  source 1..58: mol_type = other DNA
gagtttttttttcttttttcttttttttcttcttttttttgtgctaacaaagacggca

SEQ ID NO: 49 - DNA, 54 bp, Synthetic construct
  source 1..54: mol_type = other DNA
gagtttttcttttttcttttttttcttcttttttttgtgctaacaaagacggca

SEQ ID NO: 50 - DNA, 58 bp, Synthetic construct
  source 1..58: mol_type = other DNA
gagtttttcttttatttaaaaattttcttattttaaaattgtgctaacaaagacggca

SEQ ID NO: 51 - DNA, 60 bp, Synthetic construct
  source 1..60: mol_type = other DNA
cccccttttttttttttttttttttttttttttttttttttcaggaaggtgaaggtgagg

SEQ ID NO: 52 - DNA, 60 bp, Synthetic construct
  source 1..60: mol_type = other DNA
ccccctttttttttttttttttcccttttttttttttttttcaggaaggtgaaggtgagg

SEQ ID NO: 53 - DNA, 60 bp, Synthetic construct
  source 1..60: mol_type = other DNA
cccccttttttcctttttttttcccttttttttttttttttcaggaaggtgaaggtgagg

SEQ ID NO: 54 - DNA, 60 bp, Synthetic construct
  source 1..60: mol_type = other DNA
cccccttttttccttcttctttcccttttttttttttttttcaggaaggtgaaggtgagg

SEQ ID NO: 55 - DNA, 60 bp, Synthetic construct
  source 1..60: mol_type = other DNA
cccccttctttccttcttctttccctttttttttttcttttcaggaaggtgaaggtgagg

SEQ ID NO: 56 - DNA, 60 bp, Synthetic construct
  source 1..60: mol_type = other DNA
cccccttttttttttttttcttttttttttttcttttttttcaggaaggtgaaggtgagg

SEQ ID NO: 57 - DNA, 60 bp, Synthetic construct
  source 1..60: mol_type = other DNA
cccccttctttccttcttctttccctttttttttttctggtcaggaaggtgaaggtgagg

SEQ ID NO: 58 - DNA, 60 bp, Synthetic construct
  source 1..60: mol_type = other DNA
tcacctttttttttttttttttttttttttttttttttttccaggaaggtgaaggtgagg

SEQ ID NO: 59 - DNA, 56 bp, Synthetic construct
  source 1..56: mol_type = other DNA
tcaccttttttttttttttttttttttttttttttccaggaaggtgaaggtgagga

SEQ ID NO: 60 - DNA, 60 bp, Synthetic construct
  source 1..60: mol_type = other DNA
ttcaccttttttttttatatattttttttttttttttttccaggaaggtgaaggtgagga

SEQ ID NO: 61 - DNA, 59 bp, Synthetic construct
  source 1..59: mol_type = other DNA
cccccttttttttttttttttttttttttttttttttttttgcgtagaagccttttggc

SEQ ID NO: 62 - DNA, 59 bp, Synthetic construct
  source 1..59: mol_type = other DNA
ccccctttttttttttttttttcccttttttttttttttttgcgtagaagccttttggc

SEQ ID NO: 63 - DNA, 59 bp, Synthetic construct
  source 1..59: mol_type = other DNA
cccccttttttcctttttttttcccttttttttttttttttgcgtagaagccttttggc

SEQ ID NO: 64 - DNA, 59 bp, Synthetic construct
  source 1..59: mol_type = other DNA
cccccttttttccttcttctttcccttttttttttttttttgcgtagaagccttttggc

SEQ ID NO: 65 - DNA, 59 bp, Synthetic construct
  source 1..59: mol_type = other DNA
cccccttctttccttcttctttccctttttttttttcttttgcgtagaagccttttggc

SEQ ID NO: 66 - DNA, 59 bp, Synthetic construct
  source 1..59: mol_type = other DNA
cccccttttttttttttttcttttttttttttcttttttttgcgtagaagccttttggc

SEQ ID NO: 67 - DNA, 59 bp, Synthetic construct
  source 1..59: mol_type = other DNA
cccccttctttccttcttctttccctttttttttttctggtgcgtagaagccttttggc

SEQ ID NO: 68 - DNA, 59 bp, Synthetic construct
  source 1..59: mol_type = other DNA
tcacctttttttttttttttttttttttttttttttttttcgcgtagaagccttttggc

SEQ ID NO: 69 - DNA, 54 bp, Synthetic construct
  source 1..54: mol_type = other DNA
tcaccttttttttttttttttttttttttttttttcgcgtagaagccttttggc

SEQ ID NO: 70 - DNA, 58 bp, Synthetic construct
  source 1..58: mol_type = other DNA
ttcaccttttttttttatatattttttttttttttttttcgcgtagaagccttttggc

SEQ ID NO: 71 - DNA, 12 bp, Synthetic construct
  source 1..12: mol_type = other DNA
gtcccaatagca

SEQ ID NO: 72 - DNA, 11 bp, Synthetic construct
  source 1..11: mol_type = other DNA
atagcaactct

SEQ ID NO: 73 - DNA, 12 bp, Synthetic construct
  source 1..12: mol_type = other DNA
aaaatagcaggg

(19) United States
(12) Patent Application Publication
(10) Pub. No.: US 2026/0218281 A1
(43) Pub. Date: Jul. 30, 2026

(54) HIGHLY MULTIPLEXED DIGITAL NUCLEIC ACID DETERMINATION USING MELTING TEMPERATURE

(71) Applicant: President and Fellows of Harvard College, Cambridge, MA (US)

(72) Inventors: David A. Weitz, Cambridge, MA (US); Xing Zhao, Cambridge, MA (US)

(73) Assignee: President and Fellows of Harvard College, Cambridge, MA (US)

(21) Appl. No.: 19/147,251

(22) PCT Filed: Jan. 12, 2024
(86) PCT No.: PCT/US2024/011384

§ 371 Date: Jul. 10, 2025

Related U.S. Application Data
(60) Provisional application No. 63/439,059, filed on Jan. 13, 2023.
(60) Provisional application No. 63/481,892, filed on Jan. 27, 2023.

(51) Int. Cl. C12Q 1/6844; C12Q 1/6806; C12Q 1/6813; C12Q 1/6876; C40B 40/06; C40B 70/00

(52) U.S. Cl. CPC C12Q 1/6844; C12Q 1/6806; C12Q 1/6813; C12Q 1/6876; C40B 40/06; C40B 70/00; C12Q 2600/16

(57) ABSTRACT

Methods, systems, and articles for multiplexed determination of target nucleic acids using thermal barcodes are provided. The thermal barcodes may, in some cases, be used along with one or more signaling entities for improved multiplexing. At least some methods described herein may be performed with a relatively high throughput, without sequencing of the nucleic acids, which can be expensive and slow. In some cases, the methods may be implemented using a system or article comprising a plurality of droplets comprising the target nucleic acids.

### RELATED APPLICATIONS

This application claims priority under 35 U.S.C. § 119(e) to U.S. Provisional Application No. 63/439,059, filed Jan. 13, 2023, and entitled “HIGHLY MULTIPLEXED DIGITAL NUCLEIC ACID DETERMINATION USING MELTING TEMPERATURE,” and to U.S. Provisional Application No. 63/481,892, filed Jan. 27, 2023, and entitled “HIGHLY MULTIPLEXED DIGITAL NUCLEIC ACID DETERMINATION USING MELTING TEMPERATURE,” which are incorporated herein by reference in its entirety for all purposes.

### REFERENCE TO AN ELECTRONIC SEQUENCE LISTING

The contents of the electronic sequence listing (H049870771WO00-SEQ-TC.xml; Size: 69.471 bytes; and Date of Creation: Jan. 12, 2024) is herein incorporated by reference in its entirety.

### TECHNICAL FIELD

Systems and methods for thermally barcoding and determining target nucleic acids are generally described.

### BACKGROUND

Conventional technologies permit highly specific targeting of genomic regions. However, the limitations of multiplexed amplification and barcoding constrain the size of the target panel and/or the number of samples detectable in a single assay. Targeted regions can be determined by sequencing and computational analysis, but such assays require considerable infrastructure and remain time-consuming, expensive, and complex to interpret. Simpler, faster alternatives are desirable.

### SUMMARY

Methods, systems, and articles for multiplexed determination of target nucleic acids using thermal barcodes are provided. The thermal barcodes may, in some cases, be used along with one or more signaling entities for improved multiplexing. At least some methods described herein may be performed with a relatively high throughput, without sequencing of the nucleic acids, which can be expensive and slow. In some cases, the methods may be implemented using a system or article comprising a plurality of droplets (or other compartments) comprising the target nucleic acids. The subject matter of the present disclosure involves, in some cases, interrelated products, alternative solutions to a particular problem, and/or a plurality of different uses of one or more systems and/or articles.

In one aspect, a method is provided. According to some embodiments, the method, comprises: amplifying a target nucleic acid using a barcoded primer comprising a primer sequence and a thermal barcode sequence to produce a barcoded nucleic acid, the barcoded nucleic acid comprising a sequence identical to the thermal barcode sequence and a sequence identical to the target nucleic acid, wherein the thermal barcode sequence exhibits a first peak melting point and the target nucleic acid exhibits a second peak melting point, the first peak melting point and the second peak melting point differing by at least 0.1° C.

In another aspect, a method is provided. According to some embodiments, the method comprises: amplifying a target nucleic acid using a barcoded primer comprising a primer sequence and a thermal barcode sequence to produce a barcoded nucleic acid, the barcoded nucleic acid comprising a sequence identical to the thermal barcode sequence and a sequence identical to the target nucleic acid, wherein the thermal barcode sequence has a first ratio of total nearest-neighbor binding enthalpy to total nearest-neighbor binding entropy and the target nucleic acid has a second ratio of total nearest-neighbor binding enthalpy to total nearest-neighbor binding entropy, wherein the first ratio differs from the second ratio by at least 1% of the second ratio.

In yet another aspect, a method is provided. According to some embodiments, the method comprises: amplifying a target nucleic acid using a barcoded primer comprising a primer sequence and a thermal barcode sequence to produce a barcoded nucleic acid, the barcoded nucleic acid comprising a sequence identical to the thermal barcode sequence and a sequence identical to the target nucleic acid, wherein the thermal barcode sequence comprises less than or equal to 500 nucleotides and wherein at least 55% of the nucleotides of the thermal barcode sequence are A or T or wherein at least 45% of the nucleotides of the thermal barcode are G or C, and wherein the thermal barcode sequence exhibits a peak melting temperature first peak melting point and the target nucleic acid exhibits a second peak melting point, the first peak melting point and the second peak melting point differing by at least 0.1° C.

In still another aspect, an article is provided. In some embodiments, the article comprises: a plurality of droplets comprising a first droplet and a second droplet, wherein: the first droplet comprises a first target nucleic acid and a first barcoded primer comprising a first primer sequence for the first target nucleic acid and a first thermal barcode sequence; and the second droplet comprises a second target nucleic acid and a barcoded primer comprising a second primer sequence for the second target nucleic acid and a second thermal barcode sequence; wherein a peak melting temperature of the first thermal barcode sequence differs from a peak melting temperature of the second thermal barcode sequence by at least 0.1° C.

In another aspect, an article is provided. According to some embodiments, the article comprises: a plurality of droplets comprising a first droplet and a second droplet, wherein: the first droplet comprises a first target nucleic acid and a first barcoded primer comprising a first primer sequence for the first target nucleic acid and a first thermal barcode sequence; and the second droplet comprises a second target nucleic acid and a second barcoded primer comprising a second primer sequence for the second target nucleic acid and a second thermal barcode sequence; wherein the first thermal barcode sequence has a first ratio of total nearest-neighbor binding enthalpy to total nearest-neighbor binding entropy and the second thermal barcode sequence has a second ratio of total nearest-neighbor binding enthalpy to total nearest-neighbor binding entropy, wherein the first ratio differs from the second ratio by at least 1% of the second ratio.

In yet another aspect, a method of determining a target substrate is provided. According to some embodiments, the method comprises: exposing a library of barcoded nucleic acids to the target substrate such that at least some of the barcoded nucleic acids are able to bind to the target substrate; removing barcoded nucleic acids that do not bind to the target substrate; and identifying at least some of the barcoded nucleic acids that are able to bind to the target substrate using a melting profile of at least one thermal barcode of the barcoded nucleic acids.

In still another aspect, an article is provided. According to some embodiments, the article comprises: a plurality of compartments comprising a first compartment and a second compartment, wherein: the first compartment comprises a first target nucleic acid and a first barcoded primer comprising a first primer sequence for the first target nucleic acid and a first thermal barcode sequence; and the second compartment comprises a second target nucleic acid and a barcoded primer comprising a second primer sequence for the second target nucleic acid and a second thermal barcode sequence; wherein a peak melting temperature of the first thermal barcode sequence differs from a peak melting temperature of the second thermal barcode sequence by at least 0.1° C.

In yet another aspect, an article is provided. According to some embodiments, the article comprises: a plurality of compartments comprising a first compartment and a second compartment, wherein: the first compartment comprises a first target nucleic acid and a first barcoded primer comprising a first primer sequence for the first target nucleic acid and a first thermal barcode sequence; and the second compartment comprises a second target nucleic acid and a second barcoded primer comprising a second primer sequence for the second target nucleic acid and a second thermal barcode sequence; wherein the first thermal barcode sequence has a first ratio of total nearest-neighbor binding enthalpy to total nearest-neighbor binding entropy and the second thermal barcode sequence has a second ratio of total nearest-neighbor binding enthalpy to total nearest-neighbor binding entropy, wherein the first ratio differs from the second ratio by at least 1% of the second ratio.

In one aspect, a method is provided. According to some embodiments, the method comprises: selectively barcoding a target nucleic acid using a thermal barcode to form a barcoded target nucleic acid, and amplifying the barcoded target nucleic acid, wherein the thermal barcode exhibits a first peak melting point and the target nucleic acid exhibits a second peak melting point, the first peak melting point and the second peak melting point differing by at least 0.1° C.

In another aspect, a method is provided. According to some embodiments, the method comprises: selectively barcoding a target nucleic acid using a thermal barcode to form a barcoded target nucleic acid, and amplifying the barcoded target nucleic acid, wherein the thermal barcode sequence has a first ratio of total nearest-neighbor binding enthalpy to total nearest-neighbor binding entropy and the target nucleic acid has a second ratio of total nearest-neighbor binding enthalpy to total nearest-neighbor binding entropy, wherein the first ratio differs from the second ratio by at least 1% of the second ratio.

In still another aspect, a method is provided. According to some embodiments, the method comprises: selectively barcoding a target nucleic acid using a thermal barcode to form a barcoded target nucleic acid, and amplifying the barcoded target nucleic acid, wherein the thermal barcode sequence comprises less than or equal to 500 nucleotides and wherein at least 55% of the nucleotides of the thermal barcode sequence are A or T or wherein at least 45% of the nucleotides of the thermal barcode are G or C, and wherein the thermal barcode sequence exhibits a peak melting temperature first peak melting point and the target nucleic acid exhibits a second peak melting point, the first peak melting point and the second peak melting point differing by at least 0.1° C.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A presents a schematic illustration of a plurality of primers, according to some embodiments;

FIG. 1B presents a schematic illustration of a plurality of primers and a target nucleic acid, according to some embodiments;

FIG. 1C presents a schematic illustration of a plurality of primers and a target nucleic acid, according to some embodiments;

FIG. 1D presents a schematic illustration of a plurality of primers and a target nucleic acid according to some embodiments;

FIG. 1E presents a schematic illustration of a plurality of primers and a target nucleic acid according to some embodiments;

FIG. 1F presents a schematic illustration of a plurality of primers and a target nucleic acid according to some embodiments;

FIG. 2A presents a schematic illustration of a plurality of barcodes comprising universal primer sites, thermal barcodes, and ligation sites, according to some embodiments;

FIG. 2B presents a schematic illustration of ligation of a target nucleic acid to form a barcoded target nucleic acid, according to some embodiments;

FIG. 2C presents a schematic illustration of ligation of a target nucleic acid to form a barcoded target nucleic acid, according to some embodiments;

FIG. 2D presents a schematic illustration of amplification of a barcoded target nucleic acid using a universal primer, according to some embodiments;

FIG. 2E presents a schematic illustration of amplification of a barcoded target nucleic acid using a universal primer, according to some embodiments;

FIG. 2F presents a schematic illustration of amplification of a barcoded target nucleic acid using a universal primer, according to some embodiments;

FIG. 3A presents a schematic illustration of a melting profile and of the behavior of target nucleic acids, according to some embodiments;

FIG. 3B presents a schematic illustration of a melting profile, according to some embodiments;

FIGS. 4A-4C provide non-limiting schematic illustrations of a barcoded nucleic acid comprising multiple thermal barcodes, according to some embodiments;

FIGS. 5A-5C present non-limiting, schematic representations of binding of auxiliary barcodes to barcoded nucleic acids, according to some embodiments;

FIG. 6 presents a schematic illustration of a method of determining a target nucleic acid, according to some embodiments;

FIG. 7 presents non-limiting melting profiles of barcoded target nucleic acids, according to some embodiments;

FIG. 8A presents non-limiting melting profiles of barcoded target nucleic acids, according to some embodiments;

FIG. 8B presents non-limiting melting profiles of barcoded target nucleic acids, according to some embodiments;

FIG. 8C presents non-limiting melting profiles of barcoded target nucleic acids, according to some embodiments;

FIG. 8D presents non-limiting melting profiles of barcoded target nucleic acids, according to some embodiments;

FIG. 9 presents non-limiting melting profiles of barcoded target nucleic acids, according to some embodiments;

FIG. 10A presents non-limiting melting profiles of barcoded target nucleic acids, according to some embodiments;

FIG. 10B presents non-limiting melting profiles of barcoded target nucleic acids, according to some embodiments;

FIG. 11A presents non-limiting melting profiles of barcoded target nucleic acids, according to some embodiments;

FIG. 11B presents non-limiting melting profiles of barcoded target nucleic acids, according to some embodiments;

FIG. 11C presents non-limiting melting profiles of barcoded target nucleic acids, according to some embodiments;

FIG. 11D presents non-limiting melting profiles of barcoded target nucleic acids, according to some embodiments;

FIG. 12 presents a non-limiting melting profile of a primer comprising multiple thermal barcodes and optional signaling entities;

FIG. 13 presents a histogram of non-limiting barcoded nucleic acids comprising a plurality of thermal barcodes based on the peak melting temperatures of the first barcode and the second barcode.

FIG. 14 presents a scatter plot of non-limiting barcoded nucleic acids comprising two thermal barcodes based on the peak melting points of the barcodes, as well as identified clusters of points in the scatter plot corresponding distinct thermal barcode combinations.

### DETAILED DESCRIPTION

The identification and treatment of nucleic acid targets is an important area of biotechnology, where improvements can yield better health outcomes for diseased subjects or provide useful genetic information about a subject. Discriminating between specific target nucleic acids and other biomolecules is a difficult task, which can limit the detection efficiency of many detection assays. The present disclosure is, according to some embodiments, directed towards method, systems and articles that may be used to selectively amplify and barcode target nucleic acids. Methods of screening for multiple target nucleic acids by thermal barcoding distinctively are provided herein, and may allow multiplexed testing of a sample for multiple nucleic acid targets. Related methods provided herein may be used for barcoding target nucleic acids based on their point of origin. For example, target nucleic acids may be barcoded to differentiate between subjects suspected of having a disease. A variety of related systems, methods, and articles are also provided.

In one, non-limiting example provided solely for the sake of illustration, a sample is collected from a subject suspected of having a disease. The sample may be a raw fluid sample (e.g., a saliva sample) that includes a first plurality of nucleic acids. Some of the nucleic acids may be off-target nucleic acids, but others may be target nucleic acids. The method may comprise forming a plurality of droplets (or other compartments) from the sample and introducing a second plurality of nucleic acids to the droplets, where the nucleic acids of the second plurality comprise a primer for a target nucleic acid (e.g., where the nucleic acids of the second plurality comprise a barcoded primer for the target nucleic acid), connected to a thermal barcode sequence. The nucleic acids of the second plurality may be configured, in some embodiments, such that the melting profile of each thermal barcode of the second plurality of nucleic acids is distinct from the melting profile of its associated primer and the other melting temperature barcodes of the second plurality of nucleic acids. This may be important, in some embodiments, for allowing a significantly larger number of nucleic acids to be distinguished from each other, e.g., based on the melting profile of the barcodes, as compared to other techniques. As discussed herein, in some cases, such thermal barcodes may allow the nucleic acids to be detected using melting temperature curves, e.g., in combination with different signaling entities or primers, which may enlarge the number of possible nucleic acids that can be distinguished.

This is now described in more detail, for example, in a nucleic acid amplification protocol. For instance, if a target nucleic acid, recognized by one of the primers, is present in the first plurality of nucleic acids, the primer will amplify the target nucleic acid and introduce the thermal barcode into the amplified target nucleic acid as a trailing sequence of nucleotides. Thus, the barcode associated with the target nucleic acid present in the sample will also be amplified during nucleic acid amplification, while any sequence unrecognized by the primer will not. The amplified thermal barcode may then be determined by performing a melting profile analysis of the amplified sample, according to some embodiments. Different droplets (or other compartments) may be used to screen for different target nucleic acids, and since the target nucleic acid within a droplet can be amplified independently of any other target nucleic acids, the assay can be broadly multiplexed to permit high throughput screening of a sample for a target nucleic acid.

Of course it should be recognized that a method described above is included solely for the purpose of illustration, and that the present disclosure is not in any way restricted to such a method. A more general description is provided below.

The present disclosure is directed, according to some embodiments, towards methods of assaying samples that comprise nucleic acids. A target nucleic acid may be present or absent within a sample (e.g., depending on whether or not a subject actually has a disease, etc.). In some embodiments, a target nucleic acid is amplified using a barcoded primer that comprises a thermal barcode and a primer. The primer may be configured to bind to at least a portion of the target nucleic acid, such that the barcoded primer may be used to amplify the target nucleic acid to produce a barcoded target nucleic acid (e.g., a nucleic acid that includes a sequence identical to the thermal barcode sequence and a sequence identical to the target nucleic acid), for example, using amplification techniques such as PCR or the like.

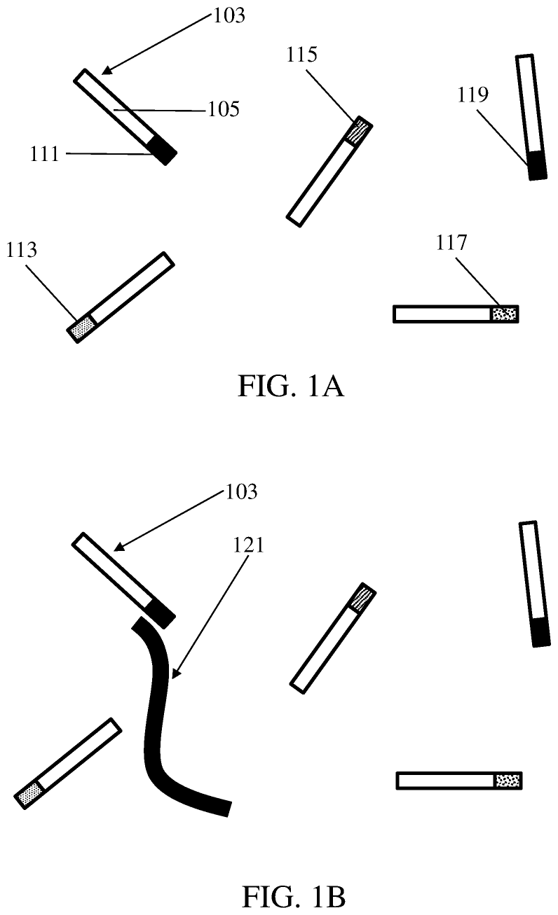

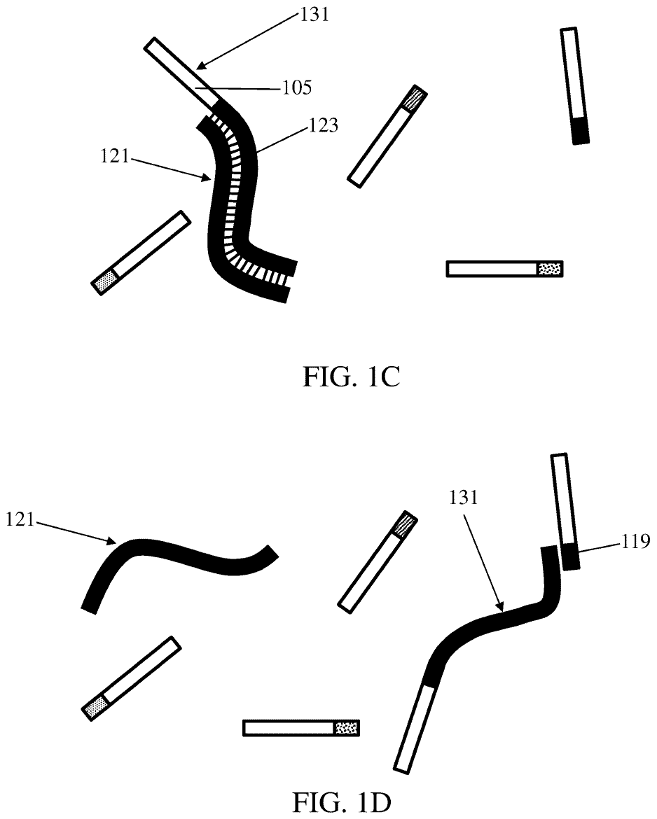

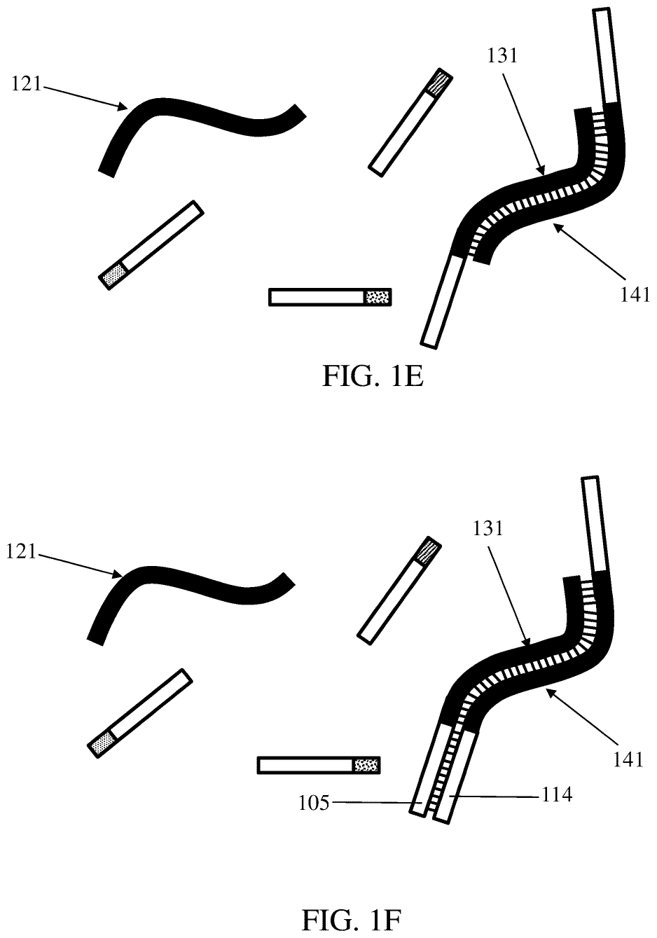

FIGS. 1A-1F present a non-limiting, schematic illustration of one method of assaying a target nucleic acid using a nucleic acid comprising thermal barcode and a primer. FIG. 1A provides a schematic illustration of a plurality of nucleic acids 103, represented as rectangles comprising thermal barcodes 105 (white rectangles), each connected to one of primers 111, 113, 115, 117, and 119 (in FIGS. 1A-1F, primers are schematically represented as shaded rectangles, with different shadings indicating different binding compatibilities with nucleic acids). Nucleic acids 103 are presented as single-stranded, but could, of course, be associated with a nucleic acid binding partner, as the disclosure is not so limited.

In FIG. 1B, nucleic acids 103 are mixed with target nucleic acid 121 (presented as curved rectangle with black shading, indicating its binding compatibility with primers 111). Amplification of target nucleic acid 121 using primer 111 is shown schematically in FIGS. 1C-1F. In FIG. 1C, nucleic acid 131 comprising target nucleic acid 123 (the complement to target nucleic acid 121) and further comprising thermal barcode 105 is formed from target nucleic acid 121 and primer 111. During subsequent steps of the amplification process, the nucleic acids may be melted and subsequently cooled, as shown in FIG. 1D, such that unused nucleic acids comprising appropriate primers and thermal barcodes have the opportunity to bind to target nucleic acids 121 or 123 in the solution. As shown in FIGS. 1E-1F, subsequent amplification steps can be used to produce more of the target nucleic acid (and/or its complement), as well as of the thermal barcode (and/or its complement). For example, FIG. 1E shows a new, barcoded target nucleic acid 141 that formed from primer 119 (as shown in FIG. 1D). During the synthesis, new nucleic acid 141 may continue to develop until it also comprises a thermal barcode strand 114 complementary to thermal barcode strand 105 of nucleic acid 131, as shown in FIG. 1F. The non-limiting example process illustrated in FIGS. 1A-1F may thus be used to synthesize and amplify nucleic acids comprising one or more thermal barcodes that may be useful for determining the target nucleic acid sequence, according to some embodiments.

It should, of course, be understood that while FIGS. 1A-1F present barcoding using a single thermal barcode, it is also possible to ligate a target nucleic acid using a plurality of thermal barcodes, auxiliary barcodes, and/or optical signaling entities, as discussed in greater detail below.

In some aspects, a target nucleic acid is barcoded and subsequently amplified using a universal primer. For example, the barcode may be part of a ligation tag—a nucleic acid configured to be ligated to form a target nucleic acid when bound to a complementary target nucleic acid sequence. Accordingly, a plurality of ligation tags comprising distinct thermal barcodes may be used to selectively barcode a plurality of target nucleic acids, if present, based on the sequences of the target nucleic acids. Thus, according to some embodiments, a plurality of ligations tags may be used to thermally barcode a plurality of target nucleic acids of interest, according to some embodiments. Ligation may be performed using any of a variety of suitable ligation reagents. In particular, in some embodiments, ligation is performed using a T4 enzyme.

According to some embodiments, the target nucleic acid is a DNA. However, in some embodiments the target nucleic acid is an RNA. When the target nucleic acid is an RNA, in some embodiments, the RNA may be barcoded by forming a barcoded target cDNA strand by any of a variety of suitable techniques (e.g., by reverse transcription). For example, in some embodiments a barcoded DNA primer may be incubated with the target RNA in a reverse transcriptase solution to produce a barcoded target cDNA, which may subsequently be determined to determine the target RNA.

In some embodiments, it is advantageous to amplify a barcoded target nucleic acid (e.g., a target nucleic acid formed by ligation or reverse transcription). In some embodiments, it is advantageous to amplify any barcoded target nucleic acids (e.g., using a DNA amplification technique such as PCR as detailed elsewhere herein). Accordingly, in some embodiments, a target nucleic acid is ligated using a ligation tag that comprises a barcode and a primer site configured to permit the barcode to be amplified by a primer. Likewise, in some embodiments, a barcoded target cDNA associated with a primer comprises a primer site configured to permit the barcode to be amplified by a primer. In some embodiments, the primer site is part of the thermal barcode. However, according to some embodiments, it may be advantageous to amplify all barcoded target nucleic acids using a universal primer (i.e., a primer configured to amplify a plurality of barcoded target nucleic acids, if present). It should, of course, be understood that multiple (e.g., complementary) universal primers may be used, depending on the embodiments. Thus, according to some embodiments, the primer site is a universal primer site of the ligation tag. A plurality of ligation tags comprising distinct thermal barcodes may each comprise the same universal primer site. The advantage of using a universal primer site is that, in some embodiments, a single primer may be used to amplify every nucleic acid comprising the universal primer site, regardless of whether every nucleic acid comprising the universal primer site comprises the same thermal barcode.

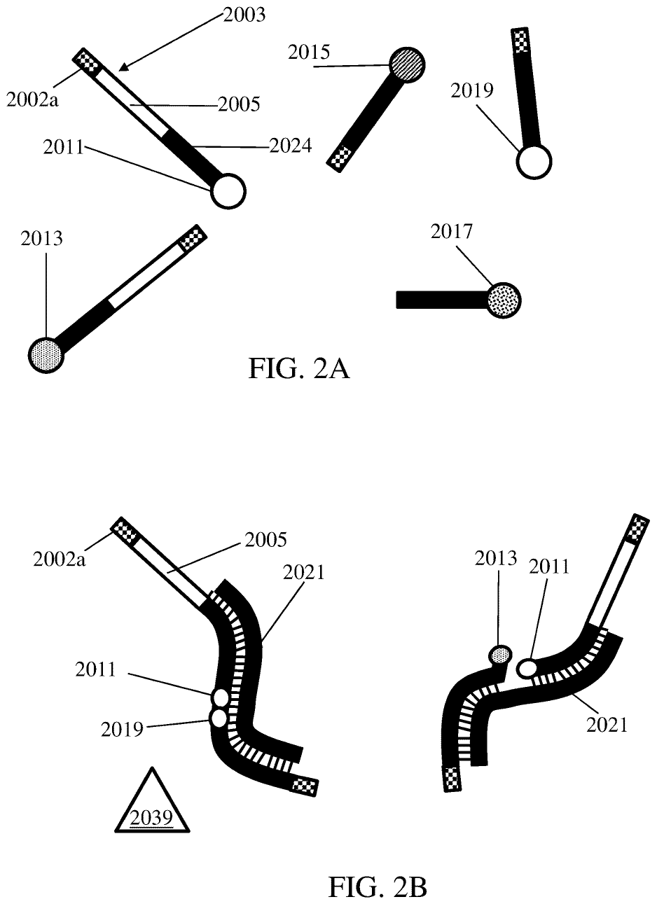

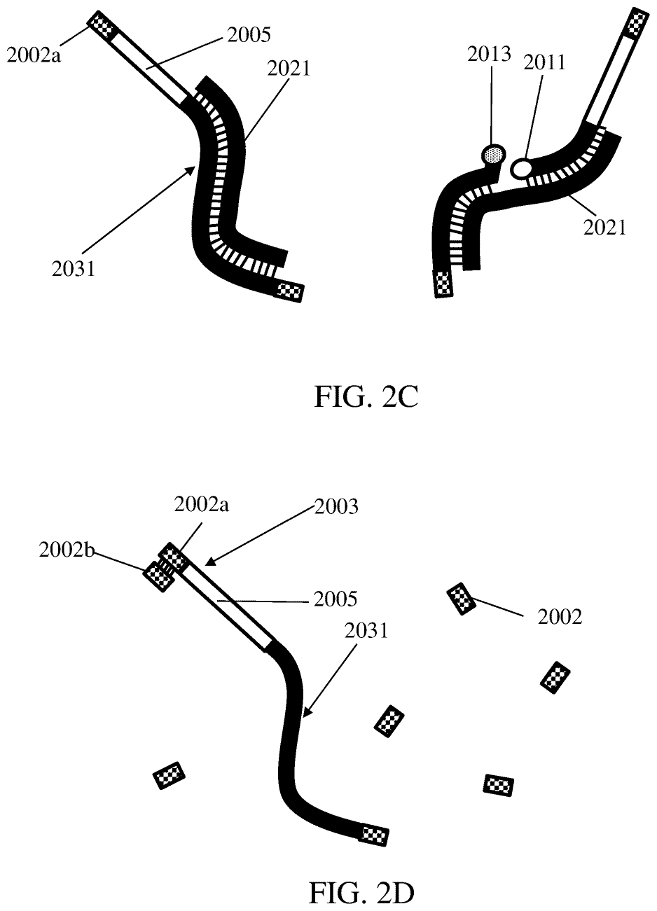

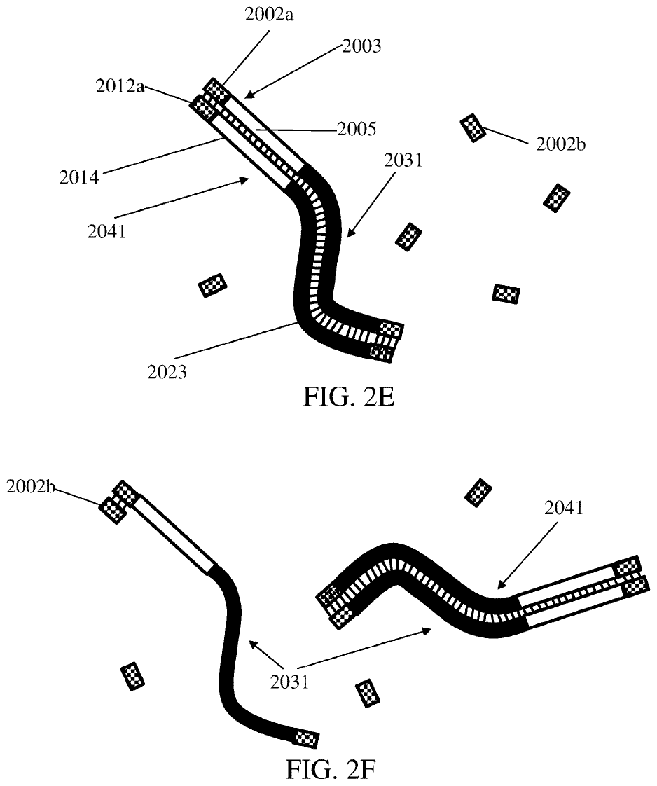

FIGS. 2A-2E present a non-limiting, schematic illustration of one method of assaying a target nucleic acid using ligation tags comprising a thermal barcode and a primer site. FIG. 2A provides a schematic illustration of a plurality of ligation tags 2003. Some of ligation tags 2003 comprise thermal barcodes 2005 (white rectangles), and the ligation tags comprise nucleic acid sequences 2024 (black curved regions) complementary to one or more target nucleic acids. Nucleic acid sequences 2024 may be configured to specifically ligate one or more target nucleic acids, e.g., by binding to a portion of one of the one or more target nucleic acids. Ligation sites 2011, 2013, 2015, 2017, and 2019 (in FIGS. 2A-2B, ligation sites are schematically represented as shaded circles, with different shadings indicating different ligation targets) may be used to specifically distinguish individual mutations in the one or more target nucleic acids to which the ligation tags are configured to bind, resulting in specific ligation of the one or more target nucleic acids. For example, the ligation site may be a terminal nucleotide of a nucleic acid sequence 2024 that is configured to bind to exactly one target nucleic acid (e.g., to exactly one mutant of a target gene), such that if ligation tag 2024 binds to an incorrect target nucleic acid its ligation site is unbound and cannot facilitate ligation.

In FIGS. 2A-2E, some ligation tags 2003 comprise universal primer sites 2002a, which are configured to be amplified using universal primer 2002b (e.g., as illustrated in FIGS. 2C-2E).

In FIG. 2B, ligation tags 2003 are mixed with target nucleic acid 2021 (presented as curved rectangle with black shading). Two target nucleic acids 2021 are shown, the first of which is bound with ligation tags comprising compatible ligation sites 2011 and 2019 and the second bound with ligation tags comprising compatible ligation site 2011 but incompatible ligation site 2013. As indicated schematically, ligation site 2013 is unbound to target nucleic acid 2021 and thus cannot ligate to ligation site 2011. In contrast, ligation tags 2011 and 2019 are both bound to adjacent portions of target nucleic acid 2021 and can thus be ligated using a ligation reagent 2039 (e.g., which may be a T4 enzyme). The result of this mixture is the formation of barcoded target nucleic acid 2031 as shown in FIG. 2C via ligation.

Amplification of barcoded target nucleic acid 2031 using universal primer 2002b is shown schematically in FIGS. 2D-2F. In FIG. 2D, nucleic acid 2031 binds to universal primer 2002b and is amplified. FIG. 2E shows the resulting nucleic acid 2041, comprising target nucleic acid 2023 (the complement to target nucleic acid 2021), thermal barcode 2014 (the compliment to thermal barcode 2005), and universal primer site 2012a is formed from target nucleic acid 2031 and universal primer 2002b. During subsequent steps of the amplification process, the nucleic acids may be melted and subsequently cooled, such that unused universal primers can bind to barcoded target nucleic acids 2031 or 2041 in the solution. As shown in FIG. 2F, subsequent amplification steps can be used to produce more of the barcoded target nucleic acid 2031 and/or its complement 2041, amplifying both the target sequence and its barcode. The non-limiting example process illustrated in FIGS. 2A-2F may thus be used to synthesize and amplify nucleic acids comprising one or more thermal barcodes that may be useful for determining the target nucleic acid sequence, according to some embodiments. It should, of course, be understood that while FIGS. 2A-2F present ligation using a single thermal barcode, it is also possible to ligate a target nucleic acid using a plurality of thermal barcodes, auxiliary barcodes, and/or optical signaling entities, as discussed in greater detail below. It should further be understood that the specific ligation technique described with reference to FIGS. 2A-2F is not limiting—other ligation techniques, such as extension ligation or ligation of complementary target nucleic acid strands with ligation tags is also possible, as the disclosure is not so limited. Likewise, other barcoding techniques may be used in place of the ligation described in FIGS. 2A-2C. For example, target nucleic acid 2031 could be produced as a target cDNA via reverse transcription of a target RNA, or via CRISPR, as described elsewhere herein, and could subsequently be amplified as illustrated in FIGS. 2D-2F.

Thermal barcodes may be used to identify nucleic acids based on their melting profile. A nucleic acid's melting profile generally refers to a distribution of an average rate of melting of the nucleic acid as a function of temperature. The melting profile of a nucleic acid or a plurality of nucleic acids may be obtained by using a binding detection agent (e.g., SYBR Green Dye or Eva Green Dye, discussed in greater detail below) as a proxy for a proportion of the nucleic acid present in a solution that is single-stranded or double-stranded. Without wishing to be bound by any particular theory, the rate of melting of the nucleic acid (e.g., the rate of de-binding of the double-stranded nucleic acid to form the single stranded nucleic acid) may be related to the rate of change in the amount of double stranded DNA and the amount of single stranded DNA present in solution, and thus may be directly related to the intensity of the signal produced by the binding detection agent.

To measure the melting profile of a nucleic acid, according to some embodiments, the nucleic acid may be heated while measuring a signal from the binding detection agent to produce an integrated melting profile. The integrated melting profile may then be differentiated (e.g., using a numerical differentiation algorithm, such as a finite difference algorithm) to determine the melting profile of the nucleic acid. The melting profile may then be rescaled (e.g., normalized to a highest measured value) to eliminate the effects of concentration and/or type of binding detection agent used in the measurement.

Generally, a nucleic acid in the presence of a complementary strand can melt from a double-stranded state to a single-stranded state over a range of temperatures during quasistatic heating. Quasistatic heating is given its ordinary meaning in the art, and may refer, in some instances, to rates of temperature change less than or equal to 10° C./s, less than or equal to 5° C./s, less than or equal to 1° C./s, less than or equal to 0.5° C./s, less than or equal to 0.2° C./s, or less than or equal to 0.1° C./s. Various aspects of the melting profile, such as the extent to which the nucleic acid is dissociated, the temperature distribution of melting rate, and a peak melting temperature (a temperature at which the rate of melting is greatest), may be estimated by methods known to those of ordinary skill in the art, some of which are discussed in the examples below.

A thermal barcode is a portion of a nucleic acid that can be used to label and subsequently determine a nucleic acid based on a melting profile of the thermal barcode. Ordinarily, naturally occurring nucleic acids melt continuously over a single temperature range, since disjointed melting of individual portions of the nucleic acid over separate temperature ranges is improbable in naturally occurring sequences. However, a thermal barcode of a barcoded nucleic acid may be selected such that the thermal barcode melts over a temperature range that is at least partially separated from the temperature range at which another portion of the barcoded nucleic acid melts. As described in greater detail below, the melting profile of a nucleic acid depends in large part on the sequence of the nucleic acid because, without wishing to be bound by any particular theory, nucleic acid melting is primarily related to local binding interactions between the nucleic acid and its complement. Thus, a thermal barcode may be designed to have an atypically high melting point by the disproportionate inclusion of high-binding-energy nucleotides, or to have an atypically low melting point by the disproportionate inclusion of low-binding-energy nucleotides within the sequence of the thermal barcode, relative to another portion of the nucleic acid.

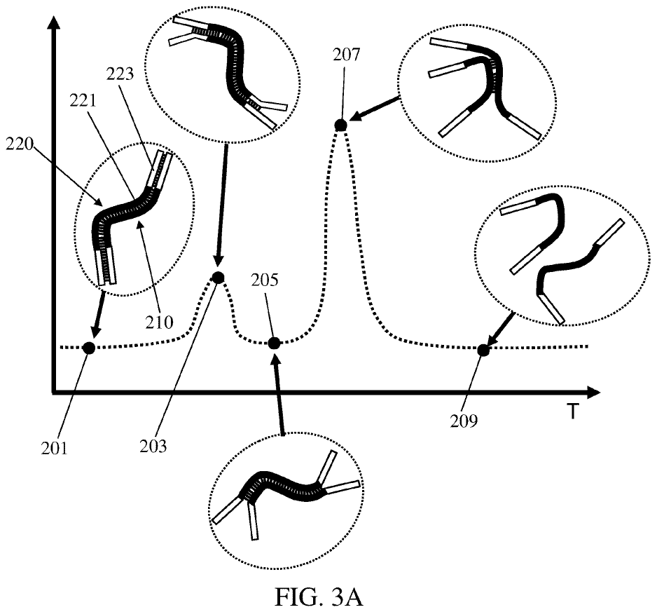

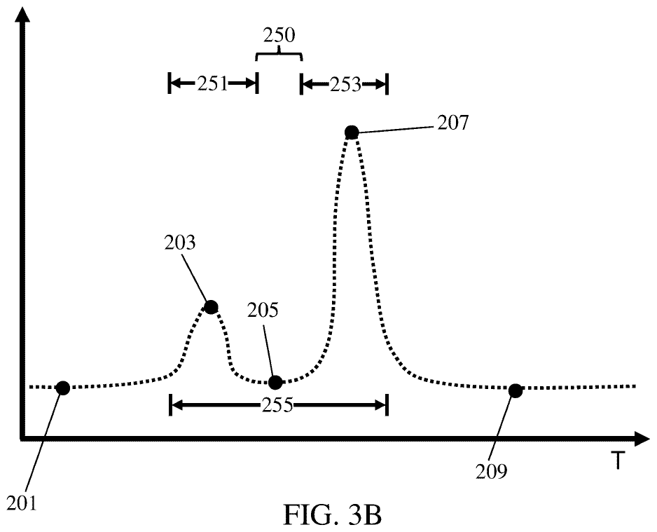

FIGS. 3A and 3B present non-limiting, schematic illustrations of an exemplary melting profile of a nucleic acid comprising a target nucleic acid and a thermal barcode during quasi-static heating, according to some embodiments. FIG. 3A presents a non-limiting melting profile showing signal intensity (corresponding to the rate of melting, along the y-axis) versus temperature (T, along the x-axis). The signal intensity of FIG. 3A is proportional to a derivative of a signal measured during a melting profile measurement, according to some embodiments. Schematic illustrations of amplified nucleic acids 210 and 220 comprising thermal barcodes 223 (curved, white rectangles) and target nucleic acids 221 (curved, black rectangles) are shown for points 201, 203, 205, 207, and 209 along the melting profile, schematically representing the condition of nucleic acids 210 and 220 at points 201, 203, 205, 207, and 209. At point 201, nucleic acids 210 and 220 are completely double-stranded and are not melting-they remain double-stranded and in quasi-steady-state (with a signal intensity of near-zero, corresponding to a melting rate of approximately zero). The thermal separation of barcode melting from target nucleic acid melting explains why the melting profile is two-peaked, rather than one-peaked. At point 205, thermal barcodes 223 have totally melted, but target nucleic acids 221 remain double-stranded, resulting in a near-zero rate of melting, and a near-zero signal intensity. Melting of target nucleic acids 221 remains near-zero at point 201, since thermal barcodes 223 were designed to melt at a lower temperature than most naturally-occurring nucleic acids, according to some embodiments. Of course, in some embodiments a melting peak of a thermal barcode at least partially overlaps with a melting peak of a target nucleic acid, so it should be understood that a point such as 205, where the signal intensity is near zero between two melting peaks, is not a necessary feature for all combinations of thermal barcodes and target nucleic acids. As the solution is heated from point 205 to point 207, the target nucleic acid begins to melt, ultimately reaching point 207, corresponding to the maximum rate of melting of the target nucleic acid. Finally, at point 209, nucleic acids 210 and 220 are fully melted, so that no further melting can occur with additional heating, and the signal intensity is near zero.

The temperature-dependent melting profile of a thermal barcode sequence may differ from a temperature-dependent melting profile of a target nucleic acid (e.g., the melting profiles of the thermal barcode and the target nucleic acid may differ by design). For example, in some embodiments, a thermal barcode sequence melts within a first temperature range and a target nucleic acid melts within a second temperature range different from the first temperature range. For example, referring to FIG. 3B (showing the same melting profile presented in FIG. 3A), the thermal barcode melts within first temperature range 251 and the target nucleic acid melts within second temperature range 253. Range 255 represents a range associated with the entire melting profile, beginning at a temperature where the nucleic acids are substantially double-stranded and ending at a temperature where they have substantially melted.

A first temperature range, wherein a thermal barcode melts, and a second temperature range, wherein a target nucleic acid melts, may at least partially overlap. Alternatively, the first temperature range, and the second temperature range may be separated such that the thermal barcode and the target nucleic acid would not melt simultaneously at any temperature during quasistatic heating. For example, in some embodiments the first temperature range is totally below the second temperature range, such that the thermal barcodes totally melt prior to any melting of the target nucleic acid during quasistatic heating. Referring again to FIG. 3B, first temperature range 251 does not overlap with second temperature range 253, meaning that the proportion of the thermal barcode that has melted and the proportion of the target nucleic acid that has melted does not substantially depend on temperature for at least one temperature between the first temperature range and the second temperature range (e.g., at point 205 between first temperature range 251 and second temperature range 253, the thermal barcode has already substantially melted but the target nucleic acid does not become more melted more as in response to a marginal increase in temperature). In some embodiments the first temperature range is totally above the second temperature range, such that the target nucleic totally melts prior to any melting of the thermal barcode during quasistatic heating. The first temperature range and the second temperature range may be separated by a temperature gap. For example, referring again to FIG. 3B, first temperature range 251 and second temperature range 253 are separated by temperature gap 250. In some embodiments, the first temperature range and the second temperature range are separated by greater than or equal to 0.1° C., greater than or equal to 0.5° C., greater than or equal to 1° C., greater than or equal to 2° C., greater than or equal to 3° C., greater than or equal to 5° C., greater than or equal to 8° C., greater than or equal to 10° C., greater than or equal to 15° C., greater than or equal to 25° C., or greater. In some embodiments, the first temperature range and the second temperature range are separated by less than or equal to 50° C., less than or equal to 25° C., less than or equal to 15° C., less than or equal to 10° C., less than or equal to 8° C., less than or equal to 5° C., less than or equal to 3° C., or less. Combinations of these ranges are possible. For example, in some embodiments, the first temperature range and the second temperature range are separated by greater than or equal to 0.1° C. and less than or equal to 50° C. Other ranges are also possible. For example, in some embodiments, the first temperature range and the second temperature range overlap, such that there is no gap between them

In some embodiments, the melting profile of the target nucleic acid is used in combination with the melting profile of the thermal barcode to identify the target nucleic acid. For example, a first target nucleic acid and a second target nucleic acid may have different peak melting temperatures or different distributions of melting temperatures. In some embodiments, the thermal barcode may act as an additional index that, in combination with the melting profile of the target nucleic acid itself, may help to distinguish between target nucleic acids, permitting multiplexed detection based on combinations of melting profiles. For example, referring again to FIG. 3B, overall melting profile 255, belonging to a first barcoded target nucleic acid, may be distinguished from a second melting profile belong to a second barcoded target nucleic acid (not shown), as long as the second barcoded target nucleic acid does not include both a thermal barcode with a melting profile similar to melting profile 251 and a target nucleic acid with a melting profile similar to melting profile 253. In this way, large numbers of target nucleic acids may be determined, even using only a small plurality of thermal barcodes, according to some embodiments.

The melting profile of a nucleic acid may be characterized in any of a variety of suitable ways. For example, a nucleic acid may have one or more peak melting temperatures. For example, referring again to FIGS. 3A-3B, points 203 and 207 represent a peak melting temperatures of thermal barcodes 223 and target nucleic acids 221, respectively. Generally, a peak melting temperature is a temperature at which a rate of melting is maximized as a function of temperature during quasistatic heating. Commonly, naturally occurring nucleic acids have one melting temperature, sometimes referred to as their melting point. However, since different portions of nucleic acids can melt over different temperature ranges, in some embodiments, a nucleic acid comprises multiple peak melting temperatures corresponding to the melting points of various portions of the nucleic acid. For example, a nucleic acid comprising thermal barcode connected to a target nucleic acid may have a first peak melting temperature corresponding to melting of the thermal barcode and a second peak melting temperature corresponding to melting of the target nucleic acid portion. The first peak melting temperature and the second peak melting temperature may be separated by any of a variety of appropriate temperature gaps (e.g., the temperature gap between points 203 and 207 in FIG. 3A). In some embodiments, the first peak melting temperature and the second peak melting temperature are separated by greater than or equal to 0.1° C., greater than or equal to 0.5° C., greater than or equal to 1° C., greater than or equal to 2° C., greater than or equal to 5° C., greater than or equal to 10° C., greater than or equal to 15° C., greater than or equal to 30° C., or greater. In some embodiments, the first peak melting temperature and the second peak melting temperature are separated by less than or equal to 60° C., less than or equal to 50° C., less than or equal to 40° C., less than or equal to 30° C., less than or equal to 20° C., less than or equal to 10° C., less than or equal to 5° C., less than or equal to 2° C., less than or equal to 1° C., or less. Combinations of these ranges are possible. For example, in some embodiments, the first peak melting temperature and the second peak melting temperature are separated by greater than or equal to 0.1° C. and less than or equal to 60° C. Other ranges are also possible.

The melting point of a nucleic acid sequence has long been understood to be related to the composition and sequence of the nucleic acid itself. For example, without wishing to be bound by any particular theory, the nearest neighbor model may be used to predict the melting point of a particular nucleic acid sequence and its perfect complementary strand based on thermodynamic parameters associated with adjacent pairs of nucleotides within the sequence. For example, Table 1 summarizes the relevant energetic parameters that may be used in calculating the melting point of a DNA sequence.

TABLE 1
Nearest-neighbor model energetic
parameters for DNA.

| Nearest-neighbor | | |
|---|---|---|
| sequence | ΔH° | ΔS° |
| (5′-3'/3'-5') | (kcal/mol) | (kcal/mol·K) |
| AA/TT or TT/AA | −9.1 | −0.0240 |
| AT/TA | −8.6 | −0.0239 |
| TA/AT | −6.0 | −0.0169 |
| CA/GT or TG/AC | −5.8 | −0.0129 |
| GT/CA or AC/TG | −6.5 | −0.0173 |
| CT/GA or AG/TC | −7.8 | −0.0208 |
| GA/CT or TC/AG | −5.6 | −0.0135 |
| CG/GC | −11.9 | −0.0278 |
| GC/CG | −11.1 | −0.0267 |
| GG/CC or CC/GG | −11.0 | −0.0266 |

The nearest-neighbor melting temperature of a sequence can generally be determined by totaling the enthalpy of each pair of nearest neighbors to determine ΔH°ₜₒₜₐₗ, totaling the entropy of each pair of nearest neighbors to determine ΔS°ₜₒₜₐₗ, and determining the melting temperature, Tₘ in temperature (Celsius), by equation (1):

$${T}_{m} = \frac{Δ {H}_{total}^{°}}{A + {S}_{total}^{°} + R ln C / 4} - 273.15 + 16.6 log [ {Na}^{+} ] \qquad ( 1 )$$

where C is the total molar DNA concentration, R is the universal gas constant of 0.00199 kcal/(K·mol), A is an initiation constant of −0.0108 kcal/(K·mol), and [Na⁺] is the molar concentration of sodium ions. For example, the nearest-neighbor melting point of the DNA sequence 5′-CGTTGA-3′ (which binds to complementary strand 3′-GCAACT-5′) can be calculated by finding the total enthalpy (ΔH°ₜₒₜₐₗ=ΔH°_{CG/GC}+ΔH°_{GT/CA}+ΔH°_{TT/AA}+ΔH°_{TG/AC}+ΔH°_{GA/CT} (−11.9)+(−6.5)+(−9.1)+(−5.8)+(−5.6)=−38.9 kcal/mol) and the total entropy (ΔS°ₜₒₜₐₗ ΔS°_{CG/GC}+ΔS°_{GT/CA}+ΔS°_{TT/AA}+ΔS°_{TG/AC}+ΔS°_{GA/CT} (−0.0278)+(−0.0173)+(−0.0240)+(−0.0129)+(−0.0135)=−0.0955 kcal/mol-K). Assuming a sodium ion concentration of 5 M and a 500 nanomolar DNA concentration, the nearest neighbor melting temperature may be determined using equation 1 to be:

$${T}_{m} = \frac{- 38.9}{- 0.0108 + ( - 0.0955 ) + 0.00199 ln \frac{5. \times {10}^{- 7}}{4}} - 273.15 + 16.6 log \qquad ( 5 )$$

The melting point, calculated by this method, is Tₘ=20.5° C.

A nucleic acid or a portion of a nucleic acid may be characterized in terms of parameters of a nearest-neighbor model as described above, in some embodiments. For example, in some embodiments, it may be advantageous to characterize a nucleic acid or nucleic acid portion directly in terms of a ratio of total nearest-neighbor binding enthalpy to total nearest-neighbor binding entropy (ΔH°ₜₒₜₐₗ/ΔS°ₜₒₜₐₗ). Without wishing to be bound by any particular theory, assuming constant salt conditions, this quantity may approximately scale with melting point of the nucleic acid or nucleic acid portion.

A first ratio of total nearest-neighbor binding enthalpy to total nearest-neighbor binding entropy of thermal barcode and a second ratio of total nearest-neighbor binding enthalpy to total nearest-neighbor binding entropy of a target nucleic acid may differ by any of a variety of appropriate amounts, such that the thermal barcode has a different melting profile than the target region. For example, in some embodiments, the first ratio and the second ratio differ by greater than or equal to 1%, greater than or equal to 2%, greater than or equal to 3%, greater than or equal to 5%, greater than or equal to 10%, greater than or equal to 12%, or greater, versus the value of the second ratio. In some embodiments, in some embodiments, the first ratio and the second ratio differ by less than or equal to 15%, less than or equal to 12%, less than or equal to 10%, less than or equal to 5%, less than or equal to 3%, less than or equal to 2%, or less, versus the value of the second ratio. Combinations of these ranges are possible. For example, in some embodiments, in some embodiments, the first ratio and the second ratio differ by greater than or equal to 1% and less than or equal to 15%, versus the value of the second ratio. Other ranges are also possible.

Of course, thermal barcodes need not be perfectly complementary to their binding partner to have predictable melting points, and in general, the melting points and melting profiles of thermal barcodes (or of nucleic acids in general) may be predicted from their sequence using software known to those of ordinary skill in the art. For example, melting profiles may be reliably predicted using software such as uMelt℠. Thus, thermal barcodes may be designed, given a target nucleic acid, such that the melting profile of the thermal barcode differs from the melting profile of the target nucleic acid as described above.

A thermal barcode to have a nucleotide sequence that is atypical of naturally occurring nucleic acids, e.g., because such a sequence could help to distinguish a melting profile of a thermal barcode from a melting profile of a target nucleic acid. For example, the thermal barcode may include a high proportion of A, T, or U, since these nucleobases typically have a weaker bonding energy than G or C.

Without wishing to be bound by any particular theory, the lower bonding energy of A, T, and U make them easier to melt, and their inclusion in an atypically high proportion in the thermal barcode can help separate the melting profile of the thermal barcode to temperatures below the melting profile of most target nucleic acids. For example, in some embodiments, the thermal barcode comprises A, T, or U (e.g., A or T) in an amount greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 98%, greater than or equal to 99%, or more of the total number of nucleotides in the thermal barcode. For example, it may be advantageous for the barcode to include greater than or equal to 70% A, T, or U, in some embodiments. Of course, A, T, or U may be included in smaller proportions, as the disclosure is not so limited.

Conversely, in some embodiments, a thermal barcode comprises G or C in an amount greater than or equal to 45%, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 98%, greater than or equal to 99%, or more of the total number of nucleotides in the thermal barcode. For example, it may be advantageous for the thermal barcode to include greater than or equal to 65% G or C, in some embodiments. Without wishing to be operated particular theory, the higher bonding energy of G and C make them harder to melt, and their inclusion in an atypically high proportion in the thermal barcode can help separate the melting profile of the thermal barcode to temperatures above the melting profile of most target nucleic acids. Of course, G or C may be included in smaller proportions, as the disclosure is not so limited.

The melting profile of a thermal barcode may be useful, for example, for the purpose of detecting a target nucleic acid. For example, in some embodiments, the presence or absence of a target nucleic acid may be determined by amplifying the nucleic acid using barcoded primers, and determining the barcode (e.g., by detecting a melting profile of the barcode within an amplified sample). As another example, in some embodiments, the presence or absence of a target nucleic acid may be determined by barcoding the target nucleic acid (e.g., via ligation, reverse transcription, CRISPR, etc.) and amplifying the barcoded nucleic acid. In some embodiments, a target nucleic acid is amplified (as discussed in greater detail below, e.g., using PCR) using a primer comprising a thermal barcode. In some embodiments, the target nucleic acid is amplified using a primer without a thermal barcode (e.g., using a universal primer). During the amplification process, both the target nucleic acid and the thermal barcode may be amplified, such that the quantity of each is increased in a solution. Alternatively, the thermal barcode can be edited directly into the genome of an organism (e.g., using CRISPR) to barcode a target nucleic acid of the organism. The melting profile of the barcoded target nucleic acid(s) may be measured, such that the thermal barcode(s) of the barcoded nucleic acid(s) may then be detected. In some embodiments, the melting profile of the thermal barcode may be used to detect the target nucleic acid. For example, a barcoded primer may be used in a sufficiently dilute concentration that its melting is undetectable, unless it is amplified by the presence of a target nucleic acid.

A thermal barcode may have any of a variety of appropriate lengths. According to some embodiments, a thermal barcode includes greater than or equal to 5, greater than or equal to 10, greater than or equal to 15, greater than or equal to 20 nucleotides, greater than or equal to 25 nucleotides, greater than or equal to 30 nucleotides, greater than or equal to 35 nucleotides, greater than or equal to 40 nucleotides, greater than or equal to 45 nucleotides, greater than or equal to 50 nucleotides, greater than or equal to 60 nucleotides, greater than or equal to 70 nucleotides, greater than or equal to 80 nucleotides, greater than or equal to 90 nucleotides, greater than or equal to 100 nucleotides, greater than or equal to 125 nucleotides, greater than or equal to 150 nucleotides, or more. According to some embodiments, a thermal barcode includes less than or equal to 500 nucleotides, less than or equal to 400 nucleotides, less than or equal to 300 nucleotides, less than or equal to 200 nucleotides, less than or equal to 175 nucleotides, less than or equal to 150 nucleotides, less than or equal to 125 nucleotides, less than or equal to 100 nucleotides, less than or equal to 90 nucleotides, less than or equal to 80 nucleotides, less than or equal to 70 nucleotides, less than or equal to 60 nucleotides, less than or equal to 50 nucleotides, or fewer. Combinations of these ranges are also possible. For example, in some embodiments, a thermal barcode includes greater than or equal to 5 nucleotides and less than or equal to 500 nucleotides. Other ranges are also possible.

Multiple thermal barcodes may be used, in some embodiments, where it may be advantageous to be able to recognize multiple target nucleic acids. The target nucleic acid is, in some embodiments, amplified in a droplet (or other compartment) comprising a plurality of nucleic acids comprising: (i) primer sequences and (ii) a plurality of thermal barcode sequences connected to at least some of the primer sequences. For example, in some embodiments, a plurality of primers comprising a plurality of thermal barcode sequences are used to amplify a plurality of target nucleic acids, and at least some of the target nucleic acids of the plurality of target nucleic acids can be subsequently determined by identifying at least some thermal barcodes of the plurality of thermal barcodes that were amplified. In some embodiments, the target nucleic acid is amplified in a droplet (or other compartment) comprising a plurality of ligation tags comprising: (i) one or more primer sequences and (ii) a plurality of thermal barcode sequences. For example, in some embodiments, a plurality of ligation tags comprising a plurality of thermal barcode sequences are used to ligate a plurality of target nucleic acids to produce a plurality of barcoded target nucleic acids, and at least some of the barcoded target nucleic acids of the plurality of target nucleic acids can be subsequently determined by amplifying and identifying at least some thermal barcodes of the plurality of thermal barcodes of the plurality using the one or more primer sequences. In some embodiments, the target nucleic acid is amplified in a droplet (or other compartment) comprising a plurality of reverse transcription primers comprising: (i) one or more primer sequences and (ii) a plurality of thermal barcode sequences. For example, in some embodiments, a plurality of reverse transcription primers comprising a plurality of thermal barcode sequences are used to perform reverse transcription on a plurality of target RNAs to produce a plurality of barcoded target cDNAs, and at least some of the target RNAs of the plurality of target RNAs can be subsequently determined by amplifying the barcoded target cDNAs and identifying at least some thermal barcodes of the plurality of thermal barcodes of the plurality using the one or more primer sequences. Additionally or alternatively, the thermal barcodes may be determined to distinguish between nucleic acids originating from different sources (e.g., from different subjects). It may be advantageous to be able to distinguish thermal barcodes from one another, as well as from naturally-occurring nucleic acids.

A plurality of primers may comprise a plurality of thermal barcodes, and in some embodiments at least some of the thermal barcodes of the plurality of thermal barcodes are distinguishable from one another by their melting profile. Similarly, a plurality of ligation tags or reverse transcription primers may comprise a plurality of thermal barcodes, and in some embodiments at least some of the thermal barcodes of the plurality of thermal barcodes are distinguishable from one another by their melting profile. Any of the above-mentioned characteristics of melting profile may be used to distinguish between the melting profiles of distinct thermal barcodes. For example a first thermal barcodes can be distinguished by identifying a peak melting temperature of the thermal barcode, or by detecting a shape of the melting profile associated with the thermal barcode.

In some embodiments, a first thermal barcode of a plurality of thermal barcodes has a peak melting temperature that differs by greater than or equal to 0.1° C., greater than or equal to 0.2° C., greater than or equal to 0.5° C., greater than or equal to 0.8° C., greater than or equal to 1° C., greater than or equal to 1.5° C., or more from the peak melting temperature of another barcode of the plurality. On the other hand, barcodes with relatively close melting profiles may be useful since, if differences between the thermal barcodes can be resolved, a greater number of thermal barcodes may be used. In some embodiments, a first thermal barcode of a plurality of thermal barcodes has a peak melting temperature that differs by less than or equal to 5° C., less than or equal to 2° C., less than or equal to 1.5° C., less than or equal to 1° C., less than or equal to 0.8° C., less than or equal to 0.5° C., less than or equal to 0.2° C., or less from the peak melting temperature of another barcode of the plurality. Combinations of these ranges are possible. For example, in some embodiments, a first thermal barcode of a plurality of thermal barcodes has a peak melting temperature that differs by greater than or equal to 0.1° C. and less than or equal to 5° C. from the peak melting temperature of another barcode of the plurality. Other ranges are also possible.

It should, of course, be understood that the same ranges may distinguish the peak melting temperature of the first thermal barcode from the peak melting temperature of more than one other thermal barcode of the plurality. For example, the first thermal barcode may have a peak melting temperature that differs from the peak melting temperature of all other thermal barcodes of the plurality by a temperature difference described in the above-mentioned ranges.

As a specific, non-limiting example, a plurality of primers may comprise a first primer for a first target nucleic acid, wherein the first primer further comprises a first thermal barcode, and a second primer for a second target nucleic acid, wherein the second primer further comprises a second thermal barcode may be distinguished from the first thermal barcode by its melting profile. Thus, in this specific, non-limiting example, in a solution comprising the first target nucleic acid and the plurality of primers, nucleic acid amplification (e.g., PCR) will result in amplification of the first thermal barcode; in a solution comprising the second target nucleic acid plurality primers, nucleic acid amplification will result in amplification of the second thermal barcode; and because the first thermal barcode and the second thermal barcode have melting profiles that are distinguishable from one another, the presence or absence of the first target nucleic acid and the presence or absence of the second target nucleic acid may each independently be established by melting the nucleic acid resulting from nucleic acid amplification and observing the melting profile resulting therefrom. Likewise, a plurality of ligation tags may comprise a first ligation tag configured to specifically ligate a first target nucleic acid, wherein the first ligation tag comprises a first thermal barcode, and a second ligation tag configured to specifically ligate a second target nucleic acid, wherein the second primer comprises a second thermal barcode that may be distinguished from the first thermal barcode by its melting profile. And likewise, in this specific, non-limiting example, in a solution comprising the first target nucleic acid and the plurality of ligation tags, ligation followed by nucleic acid amplification (e.g., PCR) will result in amplification of the first thermal barcode; in a solution comprising the second target nucleic acid plurality primers, ligation followed by nucleic acid amplification will result in amplification of the second thermal barcode; and because the first thermal barcode and the second thermal barcode have melting profiles that are distinguishable from one another, the presence or absence of the first target nucleic acid and the presence or absence of the second target nucleic acid may each independently be established by melting the nucleic acid resulting from nucleic acid amplification and observing the melting profile resulting therefrom.

A nucleic acid (e.g., a primer, a ligation tag, or a target nucleic acid) may be barcoded with more than one thermal barcode. For example, according to some embodiments, a barcoded nucleic acid (e.g., a barcoded primer, a barcoded ligation tag, and/or a barcoded nucleic acid) comprises a first thermal barcode and a second thermal barcode. For example, in some embodiments, a barcoded nucleic acid includes, in order, a nucleic acid sequence, a first thermal barcode, and a second thermal barcode. The first thermal barcode may be directly adjacent in the sequence of the barcoded nucleic acid, according to some embodiments.

In some embodiments, a first thermal barcode of a barcoded nucleic acid has a peak melting temperature that differs by greater than or equal to 0.1° C., greater than or equal to 0.2° C., greater than or equal to 0.5° C., greater than or equal to 0.8° C., greater than or equal to 1° C., greater than or equal to 1.5° C., greater than or equal to 2° C., greater than or equal to 3° C., greater than or equal to 5° C., greater than or equal to 10° C., greater than or equal to 15° C., greater than or equal to 25° C., or more from a peak melting temperature a second thermal barcode of the barcoded nucleic acid. In some embodiments, a first thermal barcode the barcoded nucleic acid has a peak melting temperature that differs by less than or equal to 50° C., less than or equal to 30° C., less than or equal to 25° C., less than or equal to 20° C., less than or equal to 15° C., less than or equal to 10° C., or less from a peak melting temperature the second thermal barcode of the barcoded nucleic acid. Combinations of these ranges are possible. For example, in some embodiments, a first thermal barcode of a barcoded nucleic acid has a peak melting temperature that differs by greater than or equal to 0.1° C. and less than or equal to 50° C. from a peak melting temperature of a second thermal barcode of the barcoded nucleic acid. Other ranges are also possible.

According to some embodiments, the first thermal barcode and the second thermal barcode of a barcoded nucleic acid have large differences in peak melting temperature. For example, in some embodiments, the first thermal barcode and the second thermal barcode of a barcoded nucleic acid have peak melting temperatures differing by at least 10° C. In some embodiments, the nucleic acid has a melting temperature that falls between the melting temperature of the first thermal barcode and the second thermal barcode.

As discussed above, the relative melting temperatures of the first thermal barcode and the second thermal barcode of a barcoded nucleic acid may be designed by controlling the sequence of the first thermal barcode and the second thermal barcode. For example, in some embodiments, the first thermal barcode comprises A, T, or U (e.g., A or T) in an amount greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 98%, greater than or equal to 99%, or more of the total number of nucleotides in the first thermal barcode and the second thermal barcode comprises G or C in an amount greater than or equal to 45%, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 98%, greater than or equal to 99%, or more of the total number of nucleotides in the second thermal barcode. In some embodiments, the second thermal barcode comprises A, T, or U in an amount greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 98%, greater than or equal to 99%, or more of the total number of nucleotides in the second thermal barcode and the first thermal barcode comprises G or C in an amount greater than or equal to 45%, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 98%, greater than or equal to 99%, or more of the total number of nucleotides in the first thermal barcode. Other sequences may also be used for the first thermal barcode and/or the second thermal barcode, as the disclosure is not so limited.

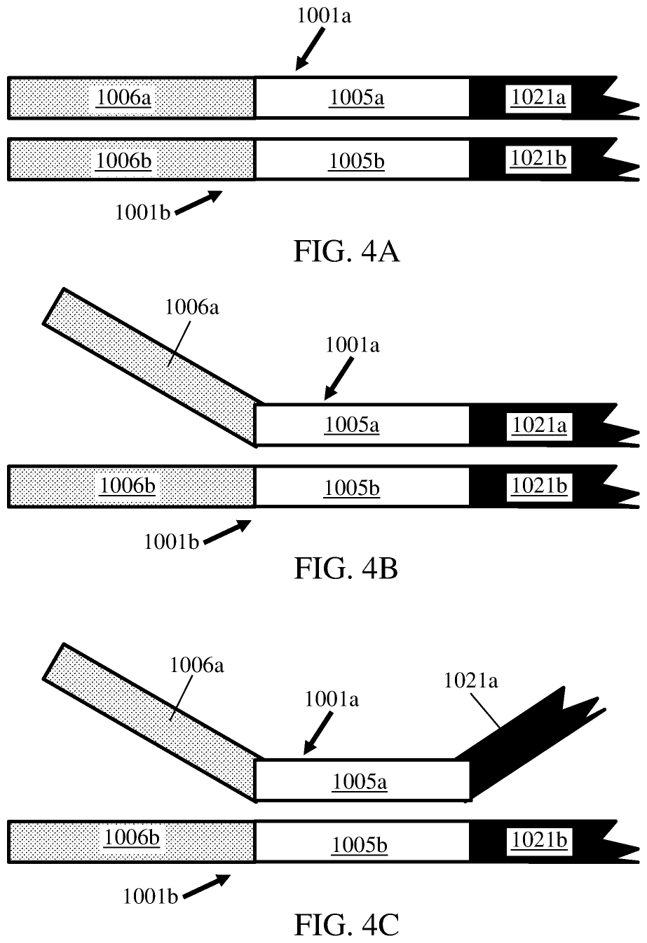

FIGS. 4A-4B provide non-limiting schematic illustrations of melting of a barcoded nucleic acid 1001a comprising first thermal barcode 1005a, second thermal barcode 1006a, and nucleic acid sequence 1021a (which may be a primer, for example).

The illustrated embodiment may be relevant where nucleic acid sequence 1021a is a primer or where nucleic acid sequence 1021a is a target nucleic acid (e.g., to which barcodes 1005a and 1006a have been attached by ligation or by amplification using barcoded primers). Also shown is a complementary barcoded nucleic acid 1001b comprising first complementary thermal barcode 1005b, second complementary thermal barcode 1006b, and complementary nucleic acid sequence 1021b, which are complementary to first thermal barcode 1005a, second thermal barcode 1006a, and nucleic acid sequence 1021a, respectively. Only a small portion of nucleic acid sequences 1021a and 1021b are shown, as indicated by the jagged cut-off of nucleic acid sequences 1021a and 1021b in the figures. FIG. 4A presents barcoded nucleic acids 1001a and 1001b at a first temperature, wherein barcoded nucleic acids 1001a and 1001b are totally double-stranded. In FIG. 4B, barcoded nucleic acids 1001a and 1001b have been heated to a second temperature, at which second thermal barcodes 1006a and 1006b have melted into single-stranded nucleic acids. In FIG. 4C, barcoded nucleic acids 1001a and 1001b have been heated to a third temperature, at which nucleic acid sequences 1021a and 1021b have melted into single-stranded nucleic acids, but first thermal barcodes 1005a and 1005b remain complexed as double-stranded DNA. Heating to an even higher temperature (not represented in these figures) would result in complete melting of nucleic acids 1001a and 1001b to form single stranded DNA. It should, of course, be understood that in some embodiments thermal barcodes may be situated on opposite ends of a target strand, rather than adjacent to the target strand as illustrated in FIGS. 4A-4C. For example, in some embodiments, primers comprising different thermal barcodes may be configured to bind to opposite ends of the target nucleic acid. As another example, in some embodiments, a first thermal barcode is ligated to a first end of the target nucleic acid and a second thermal barcode is ligated to a second end of the target nucleic acid, prior to amplification. It should also be understood that while FIGS. 4A-4C represent the melting of barcode 1006a first, the melting of nucleic acid 1021a second, and the melting of thermal barcode 1005a third, the barcodes and nucleic acid may melt in any order, depending on the embodiment. For example, it may be advantageous to choose thermal barcodes that both melt at a lower temperature or that both melt at a higher temperature than nucleic acid 1021a, depending on the embodiment.

According to some embodiments, an auxiliary barcode may be used in combination with a thermal barcode of a barcoded nucleic acid (e.g., of a barcoded primer). An auxiliary barcode is, according to some embodiments, a nucleic acid that is configured to bind to an incomplete portion of the thermal barcode of a barcoded nucleic acid. The auxiliary barcode may compete with the ability of the thermal barcode to re-solidify after melting, by binding to the incomplete portion of the thermal barcode. In some embodiments, the auxiliary barcode is configured to bind to a portion, such as an incomplete portion, of the target nucleic acid. The auxiliary barcode may compete with the ability of the target nucleic acid to re-solidify after melting, e.g., by binding to the incomplete portion of the target nucleic acid.

In some embodiments, the auxiliary barcode has a different melting profile from the thermal barcode of the thermal barcode of a barcoded nucleic acid. One advantage of using an auxiliary barcode is that, although distinct thermal barcodes may have similar melting profiles, an auxiliary barcode may be configured to bind to a particular thermal barcode sequence (e.g., by including a nucleic acid sequence complementary to the particular thermal barcode sequence). Thus, in some embodiments, the auxiliary barcode, exhibits an observable melting profile only if a thermal barcode to which it is configured to bind is present in a detectable quantity.

To provide a non-limiting example, in some embodiments, a first thermal barcode and a second thermal barcode are present in a solution (e.g., the first thermal barcode and the second thermal barcode may be bound to separate nucleic acids of a plurality of nucleic acids). The first thermal barcode and the second thermal barcode may have similar melting profiles to one another, such that it would be difficult to uniquely distinguish between the first thermal barcode and the second thermal barcode based on a melting profile, alone. According to some embodiments, an auxiliary barcode may be added to the solution, wherein the auxiliary barcode is configured to bind to a portion of the first thermal barcode but not the second thermal barcode. A melting profile of the auxiliary barcode would thus only be detectable in the presence of the first thermal barcode. Furthermore, the melting profile of the first thermal barcode would be diminished in the presence of the auxiliary barcode, due to the competitive binding of the auxiliary barcode. Thus, the first thermal barcode may be distinguished from the second thermal barcode using the auxiliary barcode.

The use of auxiliary barcodes may be particularly advantageous in the context of barcoded nucleic acids (e.g., barcoded primers) that have been barcoded with multiple thermal barcodes. Without wishing to be bound by any particular theory, in some embodiments, a single auxiliary barcode may be auxiliary to both the first thermal barcode and the second thermal barcode, e.g., by being configured to overlap a junction between the first thermal barcode and the second thermal barcode. According to some embodiments, the first thermal barcode and the second thermal barcode of the barcoded nucleic acid have large differences in peak melting temperature, as discussed above. In some embodiments, an auxiliary barcode may have a plurality of peak melting temperatures, including a peak melting temperature associated with the melting of the auxiliary barcode away from the first thermal barcode and a peak melting temperature associated with melting of the auxiliary barcode away from the second thermal barcode. The positions and relative scale of these peak melting temperatures may be controlled, in some embodiments, by changing a length and extent of overlap between the auxiliary barcode and the first thermal barcode, and/or between the auxiliary barcode and the second thermal barcode.

An auxiliary barcode may be configured to bind to any of a variety of appropriate proportions of a thermal barcode. For example, in some embodiments, an auxiliary barcode is configured to bind to greater than or equal to 5%, greater than or equal to 10%, greater than or equal to 15%, greater than or equal to 20%, greater than or equal to 25%, greater than or equal to 30%, greater than or equal to 50%, or more of the nucleotides of a thermal barcode. In some embodiments, an auxiliary barcode is configured to bind to less than or equal to 80%, less than or equal to 50%, less than or equal to 30%, less than or equal to 25%, less than or equal to 20%, less than or equal to 15%, or fewer of the nucleotides of a thermal barcode. Combinations of these ranges are possible. For example, in some embodiments, an auxiliary barcode is configured to bind to greater than or equal to 5% and less than or equal to 80% of the nucleotides of a thermal barcode. Other ranges are also possible.

An auxiliary barcode may be configured to bind to any of a variety of appropriate proportions of a target nucleic acid. For example, in some embodiments, an auxiliary barcode is configured to bind to greater than or equal to 5%, greater than or equal to 10%, greater than or equal to 15%, greater than or equal to 20%, greater than or equal to 25%, greater than or equal to 30%, greater than or equal to 40%, or more of the nucleotides of a target nucleic acid. In some embodiments, an auxiliary barcode is configured to bind to less than or equal to 80%, less than or equal to 50%, less than or equal to 40%, less than or equal to 30%, less than or equal to 25%, less than or equal to 20%, less than or equal to 15%, or fewer of the nucleotides of a target nucleic acid.

Combinations of these ranges are possible. For example, in some embodiments, an auxiliary barcode is configured to bind to greater than or equal to 5% and less than or equal to 80% of the nucleotides of a target nucleic acid. Other ranges are also possible.

Any of a variety of appropriate numbers of auxiliary barcodes may be used in combination with the thermal barcodes described herein. In some embodiments, greater than or equal to 1, greater than or equal to 2, greater than or equal to 5, greater than or equal to 10, greater than or equal to 15, greater than or equal to 20, or more auxiliary barcodes are used. In some embodiments, less than or equal to 1000, less than or equal to 500, less than or equal to 200, less than or equal to 100, less than or equal to 50, less than or equal to 20, less than or equal to 15, less than or equal to 10, less than or equal to 5, or fewer auxiliary barcodes are used. Combinations of these ranges are possible. For example, in some embodiments, greater than or equal to 1 and less than or equal to 1000 auxiliary barcodes are used. Other ranges are also possible.

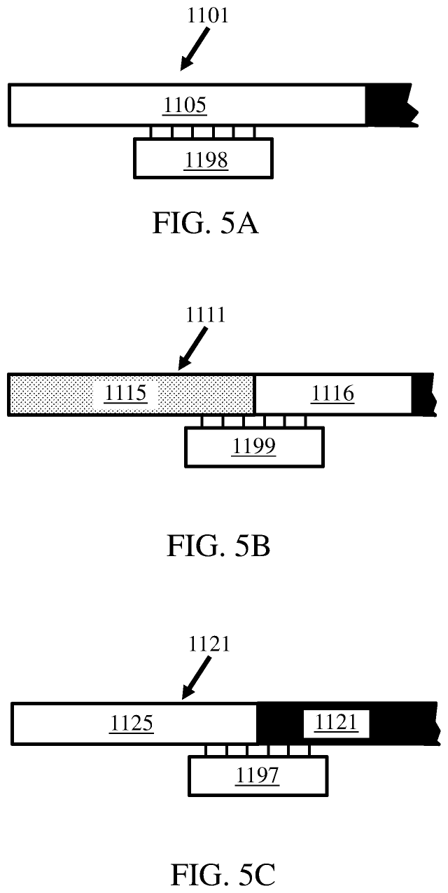

FIGS. 5A-5C present non-limiting, schematic representations of binding of auxiliary barcodes to barcoded nucleic acids, according to some embodiments. FIG. 5A presents binding of auxiliary barcode 1198 to thermal barcode 1105 of barcoded nucleic acid 1101. As shown, the auxiliary barcode is not configured to bind to all nucleotides of thermal barcode 1105, but is configured to bind to a subset of the nucleotides of thermal barcode 1105. FIG. 5B is similar to FIG. 5A, but shows an auxiliary barcode 1199 that is auxiliary to two thermal barcodes of barcoded nucleic acid 1111—first thermal barcode 1115 and second thermal barcode 1116. The melting profile of the auxiliary barcode may be designed by designing auxiliary barcode 1199 to have an appropriate overlap with thermal barcodes 1115 and 1116, as discussed in greater detail above. FIG. 5C is similar to FIGS. 5A-5B, but shows an auxiliary barcode 1197 that binds to target nucleic acid 1121 and thermal barcode 1125. As shown, auxiliary barcode 1197 may also bind to thermal barcode 1125; however, it should be understood that the auxiliary barcode can bond only to the target nucleic acid without binding to a thermal barcode, as the disclosure is not so limited.

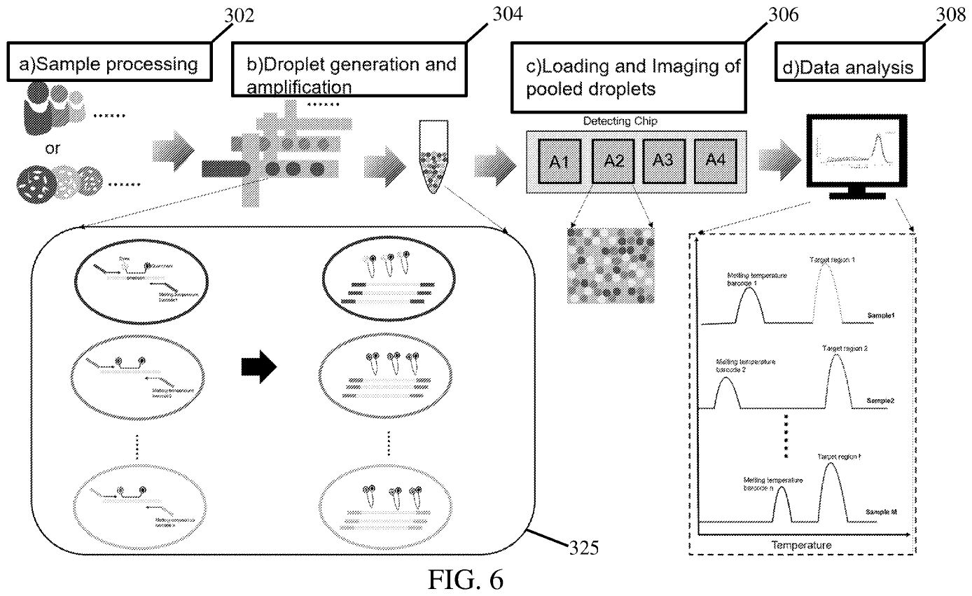

FIG. 6 presents a schematic illustration of a non-limiting method, according to some embodiments. In a first step 302, samples from a plurality of subjects and/or comprising a plurality of target nucleic acids are prepared. In a second step 304, a plurality of droplets are formed from each sample, and the nucleic acids of the droplets of the plurality of droplets are amplified (e.g., by PCR), as illustrated schematically in cutout 325, to barcode the target nucleic acid. Next, in step 306, the plurality of droplets is imaged during heating to determine the melting profile of the barcoded target nucleic acids in each droplet of the plurality. Finally, in step 308, the melting profiles of the target nucleic acids within the droplets of the plurality are analyzed to determine the nucleic acids of the droplets.

In some cases, certain primers are contained within the droplets (or other compartments) to promote amplification. Such primers may be present during formation of the droplets, and/or added to the droplets after formation of the droplets. It should be noted that the manner in which the primers are added to the droplets (or other compartments) may be the same or different from the manner in which the nucleic acids are added to the droplets (or other compartments).

A primer may be configured to bind to a target nucleic acid, if present. In some embodiments, greater than or equal to 5, greater than or equal to 10, greater than or equal to 15, greater than or equal to 20, greater than or equal to 25, greater than or equal to 30, or more consecutive nucleotides of a target nucleic acid are complimentary to complementary nucleotides of a primer. In some embodiments, less than or equal to 100, less than or equal to 75, less than or equal to 50, less than or equal to 40, less than or equal to 30, less than or equal to 25, or fewer consecutive nucleotides of a target nucleic acid are complimentary to complementary nucleotides of a primer. Combinations of these ranges are possible. For example, in some embodiments, greater than or equal to 5 and less than or equal to 100 consecutive nucleotides of a target nucleic acid are complimentary to complementary nucleotides of a primer. Other ranges are also possible.

Some or all of the primers may comprise thermal barcodes. According to some embodiments, primers without thermal barcodes may be used to amplify nucleic acids comprising thermal barcodes, thereby amplifying the thermal barcodes. This effect is advantageous, in some embodiments, since it may help ensure that thermal barcodes are only amplified if they are part of barcoded primers for a target nucleic acid that is actually present in a solution comprising the primer. However, the effect may be disadvantageous, since less thermal barcodes are likely to be produced, in total, during amplification.

In certain embodiments, a plurality of different types of primers may be added to the droplets (or other compartments). For instance, the primers may be distinguishable due to their having different sequences, and/or such that they are able to amplify different potential targets. In some cases, at least 2, at least 3, at least 5, at least 10, at least 15, at least 20, at least 25, at least 30, at least 40, at least 50, at least 60, at least 75, at least 100, at least 150, at least 200, at least 300, at least 400, at least 500, at least 1,000, at least 2,000, at least 3,000, at least 5,000, or at least 10,000, etc., different primers may be used. This may allow, for example, a variety of different target nucleic acids to be amplified within different droplets (or other compartments).

In some cases, certain ligation tags are contained within the droplets (or other compartments) to promote amplification. Such ligation tags may be present during formation of the droplets, and/or added to the droplets after formation of the droplets. It should be noted that the manner in which the ligation tags are added to the droplets (or other compartments) may be the same or different from the manner in which the nucleic acids are added to the droplets (or other compartments).

A ligation tag may be configured to bind to a specific target nucleic acid, if present. In some embodiments, greater than or equal to 5, greater than or equal to 10, greater than or equal to 15, greater than or equal to 20, greater than or equal to 25, greater than or equal to 30, or more consecutive nucleotides of a target nucleic acid are complimentary to complementary nucleotides of a ligation tag. In some embodiments, less than or equal to 100, less than or equal to 75, less than or equal to 50, less than or equal to 40, less than or equal to 30, less than or equal to 25, or fewer consecutive nucleotides of a target nucleic acid are complimentary to complementary nucleotides of a ligation tag. Combinations of these ranges are possible. For example, in some embodiments, greater than or equal to 5 and less than or equal to 100 consecutive nucleotides of a target nucleic acid are complimentary to complementary nucleotides of a ligation tag. Other ranges are also possible.

Some or all of the ligation tags may comprise thermal barcodes. In certain embodiments, a plurality of different types of ligation tags may be added to the droplets (or other compartments). For instance, the ligation tags may be distinguishable due to their having different sequences, and/or such that they are able to amplify different potential targets. In some cases, at least 2, at least 3, at least 5, at least 10, at least 15, at least 20, at least 25, at least 30, at least 40, at least 50, at least 60, at least 75, at least 100, at least 150, at least 200, at least 300, at least 400, at least 500, at least 1,000, at least 2,000, at least 3,000, at least 5,000, or at least 10,000, etc., different ligation tags may be used. This may allow, for example, a variety of different target nucleic acids to be amplified within different droplets (or other compartments).

Examples of techniques for forming droplets (or other compartments) include those described above. Examples of techniques for introducing primers after droplet formation include picoinjection or other methods such as those discussed in Int. Pat. Apl. Pub. No. WO 2010/151776, incorporated herein by reference, through fusion of the droplets with droplets containing primers, or the like. Similar methods may be used to introduce ligation tags after droplet formation. Other such techniques for either of these include, but are not limited to, any of those techniques described herein.

The primers may be present within the droplets (or other compartments) at any suitable density. The density may be independent of the density of target nucleic acids. In some cases, an excess of primers are used, e.g., such that the target nucleic acids controls the reaction. For instance, if a large excess of primers are used, then substantially all of the droplets will contain primer (regardless of whether or not the droplets also contain target nucleic acids). For example, in certain embodiments, at least about 50%, at least about 60%, at least about 70%, at least about 80%, at least about 90%, at least about 95%, at least about 97%, at least about 98%, or at least about 99% of the droplets (or other compartments) may contain at least one amplification primer.

Likewise, ligation tags may be present within the droplets (or other compartments) at any suitable density. The density may be independent of the density of target nucleic acids. In some cases, an excess of ligation tags are used, e.g., such that the target nucleic acids controls the reaction. For instance, if a large excess of ligation tags are used, then substantially all of the droplets will contain ligation tags (regardless of whether or not the droplets also contain target nucleic acids). For example, in certain embodiments, at least about 50%, at least about 60%, at least about 70%, at least about 80%, at least about 90%, at least about 95%, at least about 97%, at least about 98%, or at least about 99% of the droplets (or other compartments) may contain at least one ligation tag.

Droplets (or other compartments) containing both primer and a target nucleic acid may be treated to cause amplification of the nucleic acid to occur. This may allow a large amount or concentration of the target nucleic acids to be produced, e.g., without substantially altering the distribution of nucleic acids. In some cases, the primers are selected to allow substantially all, or only some, of the target nucleic acids suspected of being present to be amplified. Primers may be thermally barcoded or not depending on the embodiment. For example, universal primers may be used to amplify target nucleic acids barcoded using ligation tags. It should therefore be understood that the following discussion of amplification is general, and may be appropriate for embodiments with and without barcoded primers.

As examples, PCR (polymerase chain reaction) or other amplification techniques may be used to amplify nucleic acids, e.g., contained within droplets (or other compartments). Typically, in PCR reactions, the nucleic acids are heated (e.g., to a temperature of at least about 50° C., at least about 70° C., or least about 90° C. in some cases) to cause dissociation of the nucleic acids into single strands, and a heat-stable DNA polymerase (such as Taq polymerase) is used to amplify the nucleic acid. This process is often repeated multiple times to amplify the nucleic acids.

Thus, in one set of embodiments, PCR amplification may be performed within the droplets (or other compartments). For example, the droplets may contain a polymerase (such as Taq polymerase), and DNA nucleotides (deoxyribonucleotides), and the droplets may be processed (e.g., via repeated heated and cooling) to amplify the nucleic acid within the droplets. Suitable reagents for PCR or other amplification techniques, such as polymerases and/or deoxyribonucleotides, may be added to the droplets (or other compartments) during their formation, and/or afterwards (e.g., via merger with droplets containing such reagents, and/or via direct injection of such reagents, e.g., contained within a fluid). Various techniques for droplet injection or merger of droplets will be known to those of ordinary skill in the art. See, e.g., U.S. Pat. Apl. Pub. No. 2012/0132288, incorporated herein by reference. In some embodiments, primers may be added to the droplets (or other compartments), or the primers may be present on one or more of the nucleic acids within the droplets. Those of ordinary skill in the art will be aware of suitable primers, many of which can be readily obtained commercially.

Generally, any of a variety of nucleic acids may be target nucleic acids as described herein, and the disclosure is not limited to the targeting of any particular nucleic acid. As used herein, the term “target nucleic acid” may refer to a complete nucleic acid, or may refer to a portion of a larger nucleic acid that consists of a target sequence. For example, if the target nucleic acid were “ATAGCA,” any of the following nucleic acids could be targeted, but the portion of the sequence in bold-face would be considered the target nucleic acid: “ATAGCA”, “GTCCCAATAGCA” (SEQ ID NO: 71), “ATAGCAACTCT” (SEQ ID NO: 72), or “AAAATAGCAGGG” or (SEQ ID NO: 73). A target nucleic acid may be naturally occurring, in some embodiments. For example, in some embodiments a target nucleic acid is a disease indicator (e.g., an indicator of a viral, bacterial, fungal, or parasitic infection; a cancer; or a marker of any of a variety of other diseases). In some embodiments, the target nucleic acid is a portion of a subject's genome (e.g., a genetic marker). Target nucleic acids are not limited to naturally occurring sequences, and may instead be non-natural sequences (e.g., engineered sequences, randomly generated sequences, or mutants of naturally occurring nucleic acids). In some embodiments, the presence of the target nucleic acid is associated with a disease (e.g., presence of a bacterial nucleic acid). According to some embodiments, the absence of the target nucleic acid is associated with the disease (e.g., the target nucleic acid may be a gene whose absence, which could result from a genetic mutation, would negatively impact the health of an organism through its absence).

A method described herein may be sensitive to a relatively large number of target nucleic acids. In some embodiments, greater than or equal to 1, greater than or equal to 10, greater than or equal to 10², greater than or equal to 10³, greater than or equal to 10⁴, greater than or equal to 10⁵, greater than or equal to 10⁶, or more nucleic acids may be determined using a method described herein. In some embodiments, less than or equal to 10⁷, less than or equal to 10⁶, or fewer nucleic acids may be determined using a method described herein. Combinations of these ranges are possible. For example, in some embodiments, greater than or equal to 1 and less than or equal to 10⁷ nucleic acids may be determined using a method described herein. Other ranges are also possible.

A target nucleic acid is typically, but not necessarily, longer than a thermal barcode. For example, in some embodiments, a thermal barcode may be used to barcode a nucleic acid having a length that is greater than or equal to 60%, greater than or equal to 100%, greater than or equal to 150%, greater than or equal to 200%, greater than or equal to 300%, greater than or equal to 500%, greater than or equal to 1,000%, or more of a length of the thermal barcode in nucleotides. In some embodiments, a thermal barcode may be used to barcode a nucleic acid having a length that is less than or equal to 5,000%, less than or equal to 2,000%, less than or equal to 1,000%, less than or equal to 500%, less than or equal to 300%, less than or equal to 200%, or less of a length of the thermal barcode in nucleotides. Combinations of these ranges are possible. For example, in some embodiments, a thermal barcode may be used to barcode a nucleic acid having a length that is greater than or equal to 60% and less than or equal to 5,000% of a length of the thermal barcode in nucleotides. Other ranges are also possible.

When a target nucleic acid is amplified using barcoded primers, it may be determined, at least in part, by measuring the melting temperature of the barcode, as discussed in greater detail above. Similarly, when a target nucleic acid is barcoded using ligation tags and subsequently amplified, it may be determined, at least in part, by measuring the melting temperature of the barcode, as discussed in greater detail above. In some embodiments, the target nucleic acid may be determined, at least in part, by measuring the melting profile (e.g., the peak melting temperature) of both the thermal barcode and the target nucleic acid within the nucleic acids formed during nucleic acid amplification (e.g., PCR). This may be advantageous when, for example, multiple target nucleic acids may be present in the same analyte, and at least some of the target nucleic acids have different melting profiles. If a first target nucleic acid and a second target nucleic acid are known to have different melting profiles, they may each be barcoded using the same thermal barcode, since the first target nucleic acid and the second target nucleic acid may be mutually distinguished by the melting of the individual nucleic acid itself. For example, if a first target nucleic acid and a second target nucleic acid are known to have different melting profiles, they may each be amplified by primers using the same thermal barcode, since the first target nucleic acid and the second target nucleic acid may be mutually distinguished by the melting of the individual nucleic acid itself. Thus, in some embodiments, both the melting profile of the barcode and the melting profile of the target nucleic acid may be used to determine the target nucleic acid. The determination of the target nucleic acid need not be based on determination of the barcode alone, in some embodiments.

The thermal barcodes described herein may, according to some embodiments, be used for screening for a target substrate (e.g., a binding target) using a library of barcoded nucleic acids for their ability to interact with a substrate. According to some embodiments, for example, a library of barcoded nucleic acids can be prepared such by thermally barcoding the nucleic acids of the library. The barcoded nucleic acids of the library may comprise nucleic acids suitable for detection of a plurality of different possible binding targets, including the target substrate. The library of barcoded nucleic acids may be used to bind to determine (e.g., detect) a target substrate. According to some embodiments, by exposing the barcoded nucleic acids that bind to the target substrate and selecting the barcoded nucleic acids that are able to interact with that target substrate, the target substrate may be determined. For example, the method may comprise allowing the library of barcoded nucleic acids to interact with (e.g., bind to) the target substrate. In some embodiments, the melting profile of a thermal barcode bound to the target substrate may be determined. The melting profile may be used to determine the target substrate. In some embodiments, nucleic acids of the library that do not interact with the target substrate are removed (e.g., by washing the target substrate). According to some embodiments, the removal of non-binding nucleic acids increases a proportion of barcoded nucleic acids that are able to bind to the target substrate. Optionally, according to some embodiments, the nucleic acids remaining after removal of non-binding nucleic acids may be amplified. Any or all of the steps mentioned above may be iterated, to select for one or more barcoded nucleic acids that are capable of binding to the target substrate.

According to some embodiments, the one or more barcoded nucleic acids that are capable of binding to the target substrate may be identified, based at least in part on a melting profile of a thermal barcode. According to some embodiments, for example, a thermally barcoded nucleic acid of the library may be identified based, at least in part, on the melting profile of its thermal barcode. In some embodiments, one or more auxiliary barcodes are introduced to the barcoded nucleic acids. The barcoded nucleic acids of the library may thus be identified, according to some embodiments, based on the melting profile of an auxiliary barcode, as discussed in greater detail above. In some embodiments, by determining the melting profile of the thermal barcode, the identity of the associated target nucleic acid and/or the bound target substrate may be determined.

Articles and/or fluidic systems may be used to perform one or more of the methods described herein within a plurality of droplets (or other compartments). Droplet-based fluidic systems and articles may offer several advantages for determining nucleic acids. For example, microfluidic droplet-based fluidic systems and articles may facilitate high-throughput processing of samples of nucleic acids. In the context of the present disclosure, it has been recognized that the throughput and accuracy of droplet-based methods may be advantageously improved by thermal barcoding, which can help to distinguish the presence of distinct target nucleic acids, and/or to help identify a subject from which a target nucleic acid originated, as discussed in greater detail above.

Some aspects of the present disclosure are generally directed to systems and methods for containing or encapsulating nucleic acids such as those discussed herein within microfluidic droplets or other suitable compartments, for example, microwells of a microwell plate, individual spots on a slide or other surface, or the like. Thus, it should be understood that as discussed herein, when nucleic acids are contained within droplets, this is by way of example only, and in other embodiments, the nucleic acids may be contained within microwells, spots on a slide, or other suitable compartments.

A plurality of droplets generally comprises a first droplet and a second droplet, but can comprise any of an appropriate number of droplets. In some embodiments, a plurality of droplets contains greater than or equal to 5, greater than or equal to 10, greater than or equal to 10², greater than or equal to 10³, greater than or equal to 10⁴, greater than or equal to 10⁵, greater than or equal to 10⁶, greater than or equal to 10⁷, or more droplets. In some embodiments, a plurality of droplets contains less than or equal to 10⁸, less than or equal to 10⁷, less than or equal to 10⁶, less than or equal to 10⁵, or less droplets. Combinations of these ranges are possible. For example, in some embodiments, a plurality of droplets contains greater than or equal to 5 and less than or equal to 10⁸ droplets. Other ranges are also possible.

Primers, ligation tags, and/or target nucleic acids may be allocated to the droplets of the plurality in any of a variety of suitable ways. In some embodiments, it is advantageous to limit the number of target nucleic acids per droplet, while including a plurality of primers in the droplet, such that the plurality of primers may be used to uniquely identify the target nucleic acid present in each droplet. Likewise, in some embodiments, it is advantageous to limit the number of target nucleic acids per droplet, while including a plurality of ligation tags in the droplet, such that the plurality of ligation tags may be used to uniquely identify the target nucleic acid present in each droplet. This may reduce or eliminate confusion that could result from simultaneous melting of distinct thermal barcodes, amplified in the presence of distinct target nucleic acids. In some embodiments, a first plurality of droplets is formed such that greater than or equal to 80%, greater than or equal to 85%, greater than or equal to 90%, greater than or equal to 95%, greater than or equal to 98%, or more of the droplets contain either only one target nucleic acid or no target nucleic acid. In some embodiments, a first plurality of droplets is formed such that less than or equal to 100%, less than or equal to 98%, less than or equal to 95%, less than or equal to 90%, or less of the droplets contain either only one target nucleic acid or no target nucleic acid. Combinations of these ranges are possible. For example, in some embodiments, a first plurality of droplets is formed such that greater than or equal to 80% and less than or equal to 100% of the droplets contain either only one target nucleic acid or no target nucleic acid. Other ranges are also possible.

The droplets may comprise any of a variety of appropriate number of barcoded primers. In some embodiments, greater than or equal to 80%, greater than or equal to 85%, greater than or equal to 90%, greater than or equal to 95%, greater than or equal to 98%, or more of the droplets of a plurality of droplets comprise one or more barcoded primers. In some embodiments, less than or equal to 100%, less than or equal to 98%, less than or equal to 95%, less than or equal to 90%, less than or equal to 85%, or less of the droplets of a plurality of droplets comprise one or more barcoded primers. Combinations of these ranges are possible. For example, in some embodiments, greater than or equal to 80% and less than or equal to 100% of the droplets of a plurality of droplets comprise one or more barcoded primers. Other ranges are also possible. For example, the droplets may comprise no barcoded primers (e.g., if ligation tags are used).

The droplets may comprise any of a variety of appropriate number of ligation tags. In some embodiments, greater than or equal to 80%, greater than or equal to 85%, greater than or equal to 90%, greater than or equal to 95%, greater than or equal to 98%, or more of the droplets of a plurality of droplets comprise one or more ligation tags. In some embodiments, less than or equal to 100%, less than or equal to 98%, less than or equal to 95%, less than or equal to 90%, less than or equal to 85%, or less of the droplets of a plurality of droplets comprise one or more ligation tags. Combinations of these ranges are possible. For example, in some embodiments, greater than or equal to 80% and less than or equal to 100% of the droplets of a plurality of droplets comprise one or more ligation tags. Other ranges are also possible.

In some embodiments, a plurality of droplets comprises: a first droplet that comprises a first target nucleic acid and a first barcoded primer, wherein the first barcoded primer is suitable for amplifying the first target nucleic acid; and a second droplet that comprises a second target nucleic acid and a second barcoded primer, wherein the second barcoded primer is suitable for amplifying the second target nucleic acid. Thus, in some embodiments, a first target nucleic acid is detected in the first droplet and/or a second target nucleic acid is detected in a second droplet, using any of a variety of methods described above.

In some embodiments, a plurality of droplets comprises: a first droplet that comprises a first target nucleic acid and a first ligation tag, wherein the first ligation tag is suitable for amplifying the first target nucleic acid; and a second droplet that comprises a second target nucleic acid and a second ligation tag, wherein the second ligation tag is suitable for amplifying the second target nucleic acid. Thus, in some embodiments, a first target nucleic acid is detected in the first droplet and/or a second target nucleic acid is detected in a second droplet, using any of a variety of methods described above.

A plurality of droplets may be broken to the amplified nucleic acids (and amplified thermal barcodes) of the droplets. Breaking the droplets may be advantageous for any of a variety of reasons. For example breaking the droplets may allow the melting profile of a sample to be determined using a larger overall quantity of the amplified thermal barcode, which may improve detection efficiency. Of course, breaking the droplets can complicate the measurement of the temperature profile of amplified thermal barcodes, when multiple thermal barcodes are amplified within the plurality of droplets. As discussed in greater detail above, one advantage to the use of thermal barcodes with distinct melting profiles is that the thermal barcodes may be distinguished by their melting profiles, even when mixed together.

In some embodiments, a method comprises forming more than one plurality of droplets. For example, a method may comprise forming a first plurality of droplets, suspected of containing target nucleic acids from a first subject, and a second plurality of droplets, suspected of containing target nucleic acids from a second subject. Alternatively, different pluralities of droplets may be formed from the same source, but including different pluralities of barcoded primers. As another example, different pluralities of droplets may be formed from the same source, but including different pluralities of ligation tags primers. The use of multiple pluralities of droplets may permit the re-use of thermal barcodes for barcoding different target nucleic acids, since a barcoded target nucleic acid formed after amplification could be uniquely identified by knowing the barcode in combination with knowing the plurality of droplets in which the amplification of the target nucleic acid took place.

Thermal barcodes may, in some embodiments, be used in combination with other signaling entities to facilitate multiplexed detection of target nucleic acids. A signaling entity may be detected by any of a variety of suitable methods. For example, a signaling entity may be an optical signaling entity (e.g., a colorimetric signaling entity or a fluorescent signaling entity). The signaling entity may be used in any of a variety of suitable ways. For example, in some embodiments, a signaling entity may be used to indicate a subject of origin for a sample. As another example, the signaling entity may specifically indicate the presence or absence of a target nucleic acid. A signaling entity may comprise any of a variety of appropriate moieties that can be detected by a method known to one of ordinary skill. For example, the signaling entity may comprise a fluorophore, a quencher, or a dye.

Signaling entities (e.g., optical signaling entities) may be used in any of a variety of suitable ways. In some embodiments, a signaling entity does not have any specific interaction with a nucleic acid. For example, the signaling entity may simply be mixed with a sample prior to the sample's separation into droplets (or other compartments), so that the droplets can be uniquely distinguished from other droplets produced from another sample. Alternatively, a signaling entity may be designed to interact specifically with nucleic acids. For example, the signaling entity may be a modified nucleic acid, configured to emit a signal (e.g., fluoresce) in the presence of a target nucleic acid, but not in its absence. To provide a specific, non-limiting example, the signaling entity could include a fluorophore and a quencher, and could be designed such that, in the absence of a target nucleic acid, the fluorophore and the quencher are proximate, but in the presence of the target nucleic acid, the signaling entity binds to a portion of the target nucleic acid, separating the fluorophore from the quencher to produce a fluorescent signal.

As another example, a barcoded primer may be synthetically modified to include a signaling entity (e.g., an optical signaling entity such as a fluorophore). Likewise, a ligation tag may be synthetically modified to include a signaling entity. In this context, the optical signaling entity can serve as an additional mechanism for uniquely identifying a target nucleic acid. For example, in some embodiments, a plurality of barcoded primers includes a first primer connected to a first thermal barcode and a second primer connected to a second thermal barcode, wherein the first barcoded primer includes a signaling entity, the second barcoded primer does not include a signaling entity, and the first thermal barcode and the second thermal barcode are identical. In this situation, the melting profile alone might be unable to distinguish the first barcoded primer (or a nucleic acid amplified from the first barcoded primer) from the second barcoded primer (or a nucleic acid amplified from the first barcoded primer), since they include the same thermal barcode sequence and would be expected to have similar melting profiles, according to some embodiments. However, the signaling entity could be used to distinguish the first barcoded primer from the second barcoded primer by the presence or the absence of signal from the signaling entity. As another example, in some embodiments, a plurality of ligation tags includes a first ligation tag comprising a first thermal barcode and a second ligation tag comprising a second thermal barcode, wherein the first ligation tag includes a signaling entity, the second ligation tag does not include a signaling entity, and the first thermal barcode and the second thermal barcode are identical. In this situation, again, the melting profile alone might be unable to distinguish the first ligation tag (or a nucleic acid barcoded using the first ligation tag) from the second ligation tag (or a nucleic acid barcoded using the second ligation tag), since they include the same thermal barcode sequence and would be expected to have similar melting profiles, according to some embodiments. However, again, the signaling entity could be used to distinguish the first barcoded primer from the second barcoded primer by the presence or the absence of signal from the signaling entity. In this way, the presence or absence of an optical signaling entity can multiply a number of barcoded primers that can be used simultaneously, by providing an additional mechanism for distinguishing between distinct, barcoded primers.

As another example, in some embodiments one or more primers of the plurality include a signaling entity without including a thermal barcode. Thus, in some embodiments, a barcoded primer (or a nucleic acid formed therefrom) and a non-barcoded primer (or a nucleic acid formed therefrom) that both comprise the same signaling entity can be distinguished based on the presence or absence of the melting profile of the thermal barcode. Likewise, one or more ligation tags may include a signaling entity without including a thermal barcode. Thus, in some embodiments, a barcoded target nucleic acid and a non-barcoded target nucleic acid that both comprise the same signaling entity can be distinguished based on the presence or absence of the melting profile of the thermal barcode.

More than one type of signaling entity may be used, allowing for even greater multiplexing of the primers described herein. Likewise, more than one type of signaling entity may be used, allowing for even greater multiplexing of the ligation tags or target nucleic acids described herein. In some embodiments, greater than or equal to 0, greater than or equal to 1, greater than or equal to 2, greater than or equal to 3, greater than or equal to 4, greater than or equal to 5, or more signaling entities are used. In some embodiments, less than or equal to 20, less than or equal to 15, less than or equal to 10, less than or equal to 8, less than or equal to 5, less than or equal to 4, less than or equal to 3, less than or equal to 2, or fewer signaling entities are used. Combinations of these ranges are possible. For example, in some embodiments, greater than or equal to 0 and less than or equal to 20 signaling entities are used. Other ranges are also possible.

Combinations of signaling entities (e.g., melting temperature barcodes, optical signaling entities) may be used to provide any of a variety of suitable numbers of uniquely identifiable combinations suitable for distinguishing between nucleic acids. For example, in some embodiments, the number of uniquely identifiable combinations (N_{c}) is given by Equation (1):

$${N}_{C} = {a}_{1} \cdot {a}_{2} \cdot {b}^{2} \cdot N \cdot M + ( {a}_{1} + {a}_{2} ) \cdot b \cdot N \cdot M + N \cdot M \qquad ( 1 )$$

where a₁ is the number of distinguishable melting temperatures of a first thermal barcode, a₂ is the number of distinguishable melting temperatures of a second thermal barcode, b is a number of distinguishable optical signaling entities, N is a number of distinct global melting temperatures (e.g., distinct melting peaks corresponding to the overall melting profile of a first target nucleic acid), and M is a number of distinct global melting temperatures (e.g., distinct melting peaks corresponding to the overall melting profile of a second target nucleic acid). For example, if a₁=1, a₂=2, b=4, N=8 and M=8, 2880 unique combinations may be identified.

In some embodiments, a plurality of target nucleic acids may be barcoded using signaling entities that produce greater than or equal to 1, greater than or equal to 10, greater than or equal to 50, greater than or equal to 100, greater than or equal to 500, greater than or equal to 1,000, greater than or equal to 5,000, greater than or equal to 10,000, or more unique combinations of signaling entities. In some embodiments, a plurality of target nucleic acids may be barcoded using signaling entities that produce less than or equal to 100,000, less than or equal to 10,000, less than or equal to 5,000, less than or equal to 1,000, less than or equal to 500, less than or equal to 100, or less unique combinations of signaling entities. Combinations of these ranges are possible. For example, in some embodiments, a plurality of target nucleic acids may be barcoded using signaling entities that produce greater than or equal to 1 and less than or equal to 100,000 unique combinations of signaling entities. Other ranges, both higher and lower than those described above, are also possible, as the disclosure is not so limited.

Nucleic acids may be amplified by any of a variety of suitable protocols that would be known to one of ordinary skill in the art. This may be useful, for example, to produce a larger number or concentration of nucleic acids, e.g., for subsequent analysis, sequencing, or the like. Those of ordinary skill in the art will be familiar with various amplification methods that can be used, including, but are not limited to, polymerase chain reaction (PCR), reverse transcriptase (RT) PCR amplification, in vitro transcription amplification (IVT), multiple displacement amplification (MDA), multiplex ligation-dependent probe amplification (MLPA), isothermal amplification, or quantitative real-time PCR (qPCR).

In some cases, the nucleic acids may be amplified within droplets (or other compartments). This may allow amplification to occur “evenly” in some embodiments, e.g., such that the distribution of nucleic acids is not substantially changed after amplification, relative to before amplification. For example, according to certain embodiments, the nucleic acids within a plurality of droplets (or other compartments) may be amplified such that the number of nucleic acid molecules for each type of nucleic acid may have a distribution such that, after amplification, no more than about 5%, no more than about 2%, or no more than about 1% of the nucleic acids have a number less than about 90% (or less than about 95%, or less than about 99%) and/or greater than about 110% (or greater than about 105%, or greater than about 101%) of the overall average number of amplified nucleic acid molecules per droplet (or other compartment). In some embodiments, the nucleic acids within the droplets (or other compartments) may be amplified such that each of the nucleic acids that are amplified can be detected in the amplified nucleic acids, and in some cases, such that the mass ratio of the nucleic acid to the overall nucleic acid population changes by less than about 50%, less than about 25%, less than about 20%, less than about 15%, less than about 10%, or less than about 5% after amplification, relative to the mass ratio before amplification.

In some cases, certain primers are contained within the droplets (or other compartments) to promote amplification. Such primers may be present during formation of the droplets, and/or added to the droplets after formation of the droplets. Such primers may be barcoded. It should be noted that the manner in which the primers are added to the droplets (or other compartments) may be the same or different from the manner in which the nucleic acids are added to the droplets.

In certain embodiments, a plurality of different types of primers may be added to the droplets (or other compartments). For instance, the primers may be distinguishable due to their having different sequences, and/or such that they are able to amplify different potential targets. In some cases, at least 2, at least 3, at least 5, at least 10, at least 15, at least 20, at least 25, at least 30, at least 40, at least 50, at least 60, at least 75, at least 100, at least 150, at least 200, at least 300, at least 400, at least 500, at least 1,000, at least 2,000, at least 3,000, at least 5,000, or at least 10,000, etc., different primers may be used. This may allow, for example, a variety of different target nucleic acids to be amplified within different droplets (or other compartments). As discussed above, some or all of the primers may be barcoded (e.g., thermally barcoded). For example, in some embodiments, greater than or equal to 20%, greater than or equal to 30%, greater than or equal to 40%, greater than or equal to 50%, greater than or equal to 60%, greater than or equal to 70%, or more of the primers comprise thermal barcodes. In some embodiments, less than or equal to 100%, less than or equal to 90%, less than or equal to 80%, less than or equal to 70%, less than or equal to 60%, less than or equal to 50%, or less of the primers comprise thermal barcodes. Combinations of these ranges are possible. For example, in some embodiments, greater than or equal to 20% and less than or equal to 100% of the primers comprise thermal barcodes. Combinations of these ranges are possible. Other ranges are also possible.

In certain embodiments, a plurality of different types of ligation tags may be added to the droplets (or other compartments). For instance, the ligation tags may be distinguishable due to their having different sequences, and/or such that they are able to ligate different potential targets. In some cases, at least 2, at least 3, at least 5, at least 10, at least 15, at least 20, at least 25, at least 30, at least 40, at least 50, at least 60, at least 75, at least 100, at least 150, at least 200, at least 300, at least 400, at least 500, at least 1,000, at least 2,000, at least 3,000, at least 5,000, or at least 10,000, etc., different ligation tags may be used. This may allow, for example, a variety of different target nucleic acids to be amplified within different droplets (or other compartments). As discussed above, some or all of the ligation tags may be barcoded (e.g., thermally barcoded). For example, in some embodiments, greater than or equal to 20%, greater than or equal to 30%, greater than or equal to 40%, greater than or equal to 50%, greater than or equal to 60%, greater than or equal to 70%, or more of the ligation tags comprise thermal barcodes. In some embodiments, less than or equal to 100%, less than or equal to 90%, less than or equal to 80%, less than or equal to 70%, less than or equal to 60%, less than or equal to 50%, or less of the ligation tags comprise thermal barcodes. Combinations of these ranges are possible. For example, in some embodiments, greater than or equal to 20% and less than or equal to 100% of the ligation tags comprise thermal barcodes. Combinations of these ranges are possible. Other ranges are also possible.

Within the confines of a particular droplet (or other compartments), multiple primers may comprise different thermal barcodes. Such a configuration may be useful when, for example, there is a possibility that multiple target nucleic acids are present within the droplet, since the barcoded primers may be used to determine the presence of the target nucleic acids. Likewise, within the confines of a particular droplet (or other compartments), multiple ligation tags may comprise different thermal barcodes. However, the disclosure is not limited to the use of multiple thermal barcodes per droplet. In some embodiments, within the confines of a particular droplet (or other compartment), only one thermal barcode is used to label all primers. Likewise, in some embodiments, within the confines of a particular droplet (or other compartment), only one thermal barcode is used to label all ligation tags. Such a configuration may be useful when, for example, identifying the source of a target nucleic acid is desired. For example, the thermal barcode may be used to indicate that any target nucleic acids within the droplet are uniquely associated with one source (e.g., a particular subject). In this way samples determined to contain at least one target nucleic acid may be uniquely identified for further screening. Other configurations of primers within droplets are also possible, e.g., to accommodate other signaling entities (such as optical signaling entities) into a method of multiplexed detection of target nucleic acids. Likewise, other configurations of ligation tags within droplets are also possible, e.g., to accommodate other signaling entities (such as optical signaling entities) into a method of multiplexed detection of target nucleic acids.

In one set of embodiments, at least some primers may be distinguished, for example, using distinguishable fluorescent tags, barcodes, or other suitable identification tags. Examples of barcodes that can be contained within droplets include, but are not limited to, those described in U.S. Pat. Apl. Pub. No. 2018-0304222 or Int. Pat. Apl. Pub. No. WO 2015/164212, each incorporated herein by reference.

The nucleic acids may be amplified to any suitable extent. The degree of amplification may be controlled, for example, by controlling factors such as the temperature, cycle time, or amount of enzyme and/or deoxyribonucleotides contained within the droplets. For instance, in some embodiments, a population of droplets may have at least about 50,000, at least about 100,000, at least about 150,000, at least about 200,000, at least about 250,000, at least about 300,000, at least about 400,000, at least about 500,000, at least about 750,000, at least about 1,000,000 or more molecules of the amplified nucleic acid per droplet. Of course, it should be understood that amplification may occur even without multiplication of the number of molecules within the droplet. For example, the nucleic acids may be amplified using isothermal amplification, wherein the target nucleic acid (and the thermal barcode) are amplified multiple times within a nucleic acid molecule, with the result that successive amplification steps increase the length of the amplified nucleic acids.

In one set of embodiments, the droplets are broken down after amplification, e.g., to allow the amplified nucleic acids to be pooled together. A wide variety of methods for “breaking” or “bursting” droplets are available to those of ordinary skill in the art. For example, droplets contained in a carrying fluid may be disrupted using techniques such as mechanical disruption, chemical disruption, or ultrasound. Droplets may also be disrupted using chemical agents or surfactants, for example, 1H,1H,2H,2H-perfluorooctanol.

In some embodiments, a method comprises purifying nucleic acids (e.g., nucleic acids pooled from a plurality of droplets). Purification may be used, for example, to extract the nucleic acids from unwanted reagents used in earlier steps. For example, purification may be used to extract the nucleic acids from proteins transcribed therefrom. Any of a variety of appropriate techniques may be used to purify the nucleic acids. For example, the nucleic acids may be purified using any of a variety of suitable methods, such as column- or gel-based methods (including electrophoretic and centrifuge-based methods). For example, nucleic acids may be purified using a PCR clean-up kit.

After refinement of activity-producing nucleic acids, the nucleic acids may optionally be determined and/or sequenced, e.g., using techniques such as those described herein. In some embodiments, the droplets may be burst and the nucleic acids may be combined to facilitate determination and/or sequencing, although in some cases, the determination and/or sequencing may occur within the droplets.

In addition, in certain embodiments, the pool of amplified nucleic acids may be sequenced using droplet-based techniques, e.g., droplet-based PCR. For example, in some cases, the amplified nucleic acids may be collected into droplets and the droplets exposed to certain primers. In some cases, the amplified nucleic acids may be collected into droplets at relatively low concentrations, e.g., such that the droplets may, on the average, contain less than 1 nucleic acid per droplet, as described herein. In addition, in certain embodiments, the droplets may be divided into different groups of droplets, which are exposed to different primers. For instance, the droplets may be divided into at least 5, 10, 30, 100, etc. groups, which are exposed to various primers, e.g., in different spatial locations, to determine whether a target nucleic acid was present in the sample. However, it should be understood that in other embodiments, the amplified nucleic acids may be present at relatively higher concentrations, e.g., at least 1 nucleic acid per droplet or at least 1 target per droplet. In some cases, more than one primer or one amplicon may be present within a droplet.

Examples of methods for determining and/or sequencing nucleic acids include, but are not limited to, chain-termination sequencing, sequencing-by-hybridization, Maxam-Gilbert sequencing, dye-terminator sequencing, chain-termination methods, Massively Parallel Signature Sequencing (Lynx Therapeutics), polony sequencing, pyrosequencing, sequencing by ligation, ion semiconductor sequencing, DNA nanoball sequencing, single-molecule real-time sequencing (e.g., Pacbio sequencing), nanopore sequencing, Sanger sequencing, digital RNA sequencing (“digital RNA-seq”), Illumina sequencing, etc. In some cases, a microarray, such as a DNA microarray, may be used, for example, to determine, or to sequence, a nucleic acid. In some cases, the pool of amplified nucleic acids may be determined or identified, e.g., without any sequencing.

A binding detection agent may be a compound that is able to indicate whether DNA present in solution is double stranded (bound) or single stranded (unbound). A binding detection agent may be non-specific, and may be used to detect DNA binding in general, without significant dependence on the sequence of the DNA. Any of a variety of binding detection agents may be used, many of which are readily available commercially. For example, in some embodiments, a double stranded binding detection agent is used. The double stranded DNA binding detection agent may be configured such that, in the presence of double stranded DNA, the binding detection agent is activated and generates a detectable signal. The binding detection agent may be an optical detection agent, such as a dye. For example, in some embodiments, the binding detection agent is a non-specific double-strand DNA binding dye (e.g., a binSYBR Green Dye or Eva Green Dye), which is able to fluoresce upon encountering double-stranded DNA, but not upon encountering single-stranded DNA.

Additional details regarding systems and methods for manipulating droplets in a microfluidic system follow, in accordance with certain aspects. For example, various systems and methods for screening and/or sorting droplets are described in U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007, incorporated herein by reference. As a non-limiting example, in some aspects, by applying (or removing) a first electric field (or a portion thereof), a droplet may be directed to a first region or channel; by applying (or removing) a second electric field to the device (or a portion thereof), the droplet may be directed to a second region or channel; by applying a third electric field to the device (or a portion thereof), the droplet may be directed to a third region or channel; etc., where the electric fields may differ in some way, for example, in intensity, direction, frequency, duration, etc.

As mentioned, certain embodiments comprise a droplet contained within a carrying fluid. For example, there may be a first phase forming droplets contained within a second phase, where the surface between the phases comprises one or more proteins. For example, the second phase may comprise oil or a hydrophobic fluid, while the first phase may comprise water or another hydrophilic fluid (or vice versa). It should be understood that a hydrophilic fluid is a fluid that is substantially miscible in water and does not show phase separation with water at equilibrium under ambient conditions (typically 25° C. and 1 atm). Examples of hydrophilic fluids include, but are not limited to, water and other aqueous solutions comprising water, such as cell or biological media, ethanol, salt solutions, saline, blood, etc. In some cases, the fluid is biocompatible.

Similarly, a hydrophobic fluid is one that is substantially immiscible in water and will show phase separation with water at equilibrium under ambient conditions. As previously discussed, the hydrophobic fluid is sometimes referred to by those of ordinary skill in the art as the “oil phase” or simply as an oil. Non-limiting examples of hydrophobic fluids include oils such as hydrocarbons oils, silicon oils, fluorocarbon oils, organic solvents, perfluorinated oils, perfluorocarbons such as perfluoropolyether, etc. Additional examples of potentially suitable hydrocarbons include, but are not limited to, light mineral oil (Sigma), kerosene (Fluka), hexadecane (Sigma), decane (Sigma), undecane (Sigma), dodecane (Sigma), octane (Sigma), cyclohexane (Sigma), hexane (Sigma), or the like. Non-limiting examples of potentially suitable silicone oils include 2 cst polydimethylsiloxane oil (Sigma). Non-limiting examples of fluorocarbon oils include FC3283 (3M), FC40 (3M), Krytox GPL (Dupont), etc. In addition, other hydrophobic entities may be contained within the hydrophobic fluid in some embodiments. Non-limiting examples of other hydrophobic entities include drugs, immunologic adjuvants, or the like.

Thus, the hydrophobic fluid may be present as a separate phase from the hydrophilic fluid. In some embodiments, the hydrophobic fluid may be present as a separate layer, although in other embodiments, the hydrophobic fluid may be present as individual fluidic droplets contained within a continuous hydrophilic fluid, e.g. suspended or dispersed within the hydrophilic fluid. This is often referred to as an oil/water emulsion. The droplets may be relatively monodisperse, or be present in a variety of different sizes, volumes, or average diameters. In some cases, the droplets may have an overall average diameter of less than about 1 mm, or other dimensions as discussed herein. In some cases, a surfactant may be used to stabilize the hydrophobic droplets within the hydrophilic liquid, for example, to prevent spontaneous coalescence of the droplets. Non-limiting examples of surfactants include those discussed in U.S. Pat. Apl. Pub. No. 2010/0105112, incorporated herein by reference. Other non-limiting examples of surfactants include Span80 (Sigma), Span80/Tween-20 (Sigma), Span80/Triton X-100 (Sigma), Abil EM90 (Degussa), Abil we09 (Degussa), polyglycerol polyricinoleate “PGPR90” (Danisco), Tween-85, 749 Fluid (Dow Corning), the ammonium carboxylate salt of Krytox 157 FSL (Dupont), the ammonium carboxylate salt of Krytox 157 FSM (Dupont), or the ammonium carboxylate salt of Krytox 157 FSH (Dupont). In addition, the surfactant may be, for example, a peptide surfactant, bovine serum albumin (BSA), or human serum albumin.

The droplets may have any suitable shape and/or size. In some cases, the droplets may be microfluidic, and/or have an average diameter of less than about 1 mm. For instance, the droplet may have an average diameter of less than about 1 mm, less than about 700 micrometers, less than about 500 micrometers, less than about 300 micrometers, less than about 100 micrometers, less than about 70 micrometers, less than about 50 micrometers, less than about 30 micrometers, less than about 10 micrometers, less than about 5 micrometers, less than about 3 micrometers, less than about 1 micrometer, etc. The average diameter may also be greater than about 1 micrometer, greater than about 3 micrometers, greater than about 5 micrometers, greater than about 7 micrometers, greater than about 10 micrometers, greater than about 30 micrometers, greater than about 50 micrometers, greater than about 70 micrometers, greater than about 100 micrometers, greater than about 300 micrometers, greater than about 500 micrometers, greater than about 700 micrometers, or greater than about 1 mm in some cases. Combinations of any of these are also possible; for example, the diameter of the droplet may be between about 1 mm and about 100 micrometers. The diameter of a droplet, in a non-spherical droplet, may be taken as the diameter of a perfect mathematical sphere having the same volume as the non-spherical droplet.

In some embodiments, the droplets may be of substantially the same shape and/or size (i.e., “monodisperse”), or of different shapes and/or sizes, depending on the particular application. In some cases, the droplets may have a homogenous distribution of cross-sectional diameters, i.e., in some embodiments, the droplets may have a distribution of average diameters such that no more than about 20%, no more than about 10%, or no more than about 5% of the droplets may have an average diameter greater than about 120% or less than about 80%, greater than about 115% or less than about 85%, greater than about 110% or less than about 90%, greater than about 105% or less than about 95%, greater than about 103% or less than about 97%, or greater than about 101% or less than about 99% of the average diameter of the microfluidic droplets. Some techniques for producing homogenous distributions of cross-sectional diameters of droplets are disclosed in International Patent Application No. PCT/US2004/010903, filed Apr. 9, 2004, entitled “Formation and Control of Fluidic Species,” by Link, et al., published as WO 2004/091763 on Oct. 28, 2004, incorporated herein by reference. In addition, in some instances, the coefficient of variation of the average diameter of the droplets may be less than or equal to about 20%, less than or equal to about 15%, less than or equal to about 10%, less than or equal to about 5%, less than or equal to about 3%, or less than or equal to about 1%. However, in other embodiments, the droplets may not necessarily be substantially monodisperse, and may instead exhibit a range of different diameters.

Those of ordinary skill in the art will be able to determine the average diameter of a population of droplets, for example, using laser light scattering or other known techniques. The droplets so formed can be spherical, or non-spherical in certain cases. The diameter of a droplet, in a non-spherical droplet, may be taken as the diameter of a perfect mathematical sphere having the same volume as the non-spherical droplet.

In some embodiments, one or more droplets may be created within a channel by creating an electric charge on a fluid surrounded by a liquid, which may cause the fluid to separate into individual droplets within the liquid. In some embodiments, an electric field may be applied to the fluid to cause droplet formation to occur. The fluid can be present as a series of individual charged and/or electrically inducible droplets within the liquid. Electric charge may be created in the fluid within the liquid using any suitable technique, for example, by placing the fluid within an electric field (which may be AC, DC, etc.), and/or causing a reaction to occur that causes the fluid to have an electric charge.

The electric field, in some embodiments, is generated from an electric field generator, i.e., a device or system able to create an electric field that can be applied to the fluid. The electric field generator may produce an AC field (i.e., one that varies periodically with respect to time, for example, sinusoidally, sawtooth, square, etc.), a DC field (i.e., one that is constant with respect to time), a pulsed field, etc. Techniques for producing a suitable electric field (which may be AC, DC, etc.) are known to those of ordinary skill in the art. For example, in one embodiment, an electric field is produced by applying voltage across a pair of electrodes, which may be positioned proximate a channel such that at least a portion of the electric field interacts with the channel. The electrodes can be fashioned from any suitable electrode material or materials known to those of ordinary skill in the art, including, but not limited to, silver, gold, copper, carbon, platinum, copper, tungsten, tin, cadmium, nickel, indium tin oxide (“ITO”), etc., as well as combinations thereof.

In another set of embodiments, droplets of fluid can be created from a fluid surrounded by a liquid within a channel by altering the channel dimensions in a manner that is able to induce the fluid to form individual droplets. The channel may, for example, be a channel that expands relative to the direction of flow, e.g., such that the fluid does not adhere to the channel walls and forms individual droplets instead, or a channel that narrows relative to the direction of flow, e.g., such that the fluid is forced to coalesce into individual droplets. In some cases, the channel dimensions may be altered with respect to time (for example, mechanically or electromechanically, pneumatically, etc.) in such a manner as to cause the formation of individual droplets to occur. For example, the channel may be mechanically contracted (“squeezed”) to cause droplet formation, or a fluid stream may be mechanically disrupted to cause droplet formation, for example, through the use of moving baffles, rotating blades, or the like.

Some embodiments generally relate to systems and methods for fusing or coalescing two or more droplets into one droplet, e.g., where the two or more droplets ordinarily are unable to fuse or coalesce, for example, due to composition, surface tension, droplet size, the presence or absence of surfactants, etc. In certain cases, the surface tension of the droplets, relative to the size of the droplets, may also prevent fusion or coalescence of the droplets from occurring.

As a non-limiting example, two droplets can be given opposite electric charges (i.e., positive and negative charges, not necessarily of the same magnitude), which can increase the electrical interaction of the two droplets such that fusion or coalescence of the droplets can occur due to their opposite electric charges. For instance, an electric field may be applied to the droplets, the droplets may be passed through a capacitor, a chemical reaction may cause the droplets to become charged, etc. The droplets, in some cases, may not be able to fuse even if a surfactant is applied to lower the surface tension of the droplets. However, if the droplets are electrically charged with opposite charges (which can be, but are not necessarily of, the same magnitude), the droplets may be able to fuse or coalesce. As another example, the droplets may not necessarily be given opposite electric charges (and, in some cases, may not be given any electric charge), and are fused through the use of dipoles induced in the droplets that causes the droplets to coalesce. Also, the two or more droplets allowed to coalesce are not necessarily required to meet “head-on.” Any angle of contact, so long as at least some fusion of the droplets initially occurs, is sufficient. See also, e.g., U.S. patent application Ser. No. 11/698,298, filed Jan. 24, 2007, entitled “Fluidic Droplet Coalescence,” by Ahn, et al., published as U.S. Patent Application Publication No. 2007/0195127 on Aug. 23, 2007, incorporated herein by reference in its entirety.

In one set of embodiments, a fluid may be injected into a droplet. The fluid may be microinjected into the droplet in some cases, e.g., using a microneedle or other such device. In other cases, the fluid may be injected directly into a droplet using a fluidic channel as the droplet comes into contact with the fluidic channel. Other techniques of fluid injection are disclosed in, e.g., International Patent Application No. PCT/US2010/040006, filed Jun. 25, 2010, entitled “Fluid Injection,” by Weitz, et al., published as WO 2010/151776 on Dec. 29, 2010; or International Patent Application No. PCT/US2009/006649, filed Dec. 18, 2009, entitled “Particle-Assisted Nucleic Acid Sequencing,” by Weitz, et al., published as WO 2010/080134 on Jul. 15, 2010, each incorporated herein by reference in its entirety.

The following documents are each incorporated herein by reference in its entirety for all purposes: Int. Pat. Apl. Pub. No. WO 2016/168584, entitled “Barcoding System for Gene Sequencing and Other Applications,” by Weitz et al.; Int. Pat. Apl. Pub. No. WO 2015/161223, entitled “Methods and Systems for Droplet Tagging and Amplification,” by Weitz, et al.; U.S. Pat. Apl. Ser. No. 61/980,541, entitled “Methods and Systems for Droplet Tagging and Amplification,” by Weitz, et al.; U.S. Pat. Apl. Ser. No. 61/981,123, entitled “Systems and Methods for Droplet Tagging,” by Bernstein, et al.; Int. Pat. Apl. Pub. No. WO 2004/091763, entitled “Formation and Control of Fluidic Species,” by Link et al.; Int. Pat. Apl. Pub. No. WO 2004/002627, entitled “Method and Apparatus for Fluid Dispersion,” by Stone et al.; Int. Pat. Apl. Pub. No. WO 2006/096571, entitled “Method and Apparatus for Forming Multiple Emulsions,” by Weitz et al.; Int. Pat. Apl. Pub. No. WO 2005/021151, entitled “Electronic Control of Fluidic Species,” by Link et al.; Int. Pat. Apl. Pub. No. WO 2011/056546, entitled “Droplet Creation Techniques,” by Weitz, et al.; Int. Pat. Apl. Pub. No. WO 2010/033200, entitled “Creation of Libraries of Droplets and Related Species,” by Weitz, et al.; U.S. Pat. Apl. Pub. No. 2012-0132288, entitled “Fluid Injection,” by Weitz, et al.; Int. Pat. Apl. Pub. No. WO 2008/109176, entitled “Assay And Other Reactions Involving Droplets,” by Agresti, et al.; and Int. Pat. Apl. Pub. No. WO 2010/151776, entitled “Fluid Injection,” by Weitz, et al.; and U.S. Pat. Apl. Ser. No. 62/072,944, entitled “Systems and Methods for Barcoding Nucleic Acids,” by Weitz, et al.

In addition, the following are incorporated herein by reference in their entireties: U.S. Pat. Apl. Ser. No. 61/981,123 filed Apr. 17, 2014; PCT Pat. Apl. Ser. No. PCT/US2015/026338, filed Apr. 17, 2015, entitled “Systems and Methods for Droplet Tagging”; U.S. Pat. Apl. Ser. No. 61/981,108 filed Apr. 17, 2014; U.S. Pat. Apl. Ser. No. 62/072,944, filed Oct. 30, 2014; PCT Pat. Apl. Ser. No. PCT/US2015/026443, filed on Apr. 17, 2015, entitled “Systems and Methods for Barcoding Nucleic Acids”; U.S. Pat. Apl. Ser. No. 62/106,981, entitled “Systems, Methods, and Kits for Amplifying or Cloning Within Droplets,” by Weitz, et al.; U.S. Pat. Apl. Pub. No. 2010-0136544, entitled “Assay and Other Reactions Involving Droplets,” by Agresti, et al.; U.S. Pat. Apl. Ser. No. 61/981,108, entitled “Methods and Systems for Droplet Tagging and Amplification,” by Weitz, et al.; Int. Pat. Apl. Pub. No. PCT/US2014/037962, filed May 14, 2014, entitled “Rapid Production of Droplets,” by Weitz, et al.; and U.S. Provisional Patent Application Ser. No. 62/133,140, filed Mar. 13, 2015, entitled “Determination of Cells Using Amplification,” by Weitz, et al.

A fluidic system may be used to perform some or all of the method steps described above. In one aspect of the present invention, emulsions are formed by flowing two, three, or more fluids through a system of channels of a fluidic system. The fluidic system may be or comprise an article. The system or article may be a microfluidic system or article. “Microfluidic,” as used herein, refers to a device, apparatus or system including at least one fluid channel having a cross-sectional dimension (measured perpendicular to the direction of fluid flow) of less than about 1 millimeter (mm), and in some cases, a ratio of length to largest cross-sectional dimension of at least 3:1.

A “channel,” as used herein, means a feature on or in a system or article that at least partially directs flow of a fluid. The channel can have any cross-sectional shape (circular, oval, triangular, irregular, square or rectangular, or the like) and can be covered or uncovered. One or more of the channels may (but not necessarily), in cross section, have a height that is substantially the same as a width at the same point.

In embodiments where it is completely covered, at least one portion of the channel can have a cross-section that is completely enclosed, or the entire channel may be completely enclosed along its entire length with the exception of its inlet(s) and/or outlet(s). A channel may also have an aspect ratio (length to average cross sectional dimension) of at least 2:1, more typically at least 3:1, 5:1, 10:1, 15:1, 20:1, or more. An open channel generally will include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the channel may partially or completely fill the channel. In some cases where an open channel is used, the fluid may be held within the channel, for example, using surface tension (i.e., a concave or convex meniscus).

The channel may be of any size, for example, having a largest dimension perpendicular to fluid flow of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some cases the dimensions of the channel may be chosen such that fluid is able to freely flow through the article or substrate. The dimensions of the channel may also be chosen, for example, to allow a certain volumetric or linear flowrate of fluid in the channel. Of course, the number of channels and the shape of the channels can be varied by any method known to those of ordinary skill in the art. In some cases, more than one channel or capillary may be used. For example, two or more channels may be used, where they are positioned inside each other, positioned adjacent to each other, positioned to intersect with each other, etc.

The fluidic droplets within the channels may have a cross-sectional dimension smaller than about 100% of an average cross-sectional dimension of the channel, and in certain embodiments, smaller than about 90%, smaller than about 80%, about 70%, about 60%, about 50%, about 40%, about 30%, about 20%, about 10%, about 5%, about 3%, about 1%, about 0.5%, about 0.3%, about 0.1%, about 0.05%, about 0.03%, or about 0.01% of the average cross-sectional dimension of the channel.

During use, at least some processing of the droplets may be performed on an article. Thus, in some embodiments, an article comprises at least some of a plurality of droplets described above. For example, the article may comprise all droplets of a plurality of droplets. The droplets may be fluidically connected to one or more reservoirs of the fluidic system (e.g., to a pool used to form droplets, to a hydrophobic fluid used to form droplets, to a supply of a target substrate, to a supply of a signaling entity, to a supply of in vitro transcription and translation reagents, or any of a variety of other fluids described herein) via the article. For example, the droplets may be connected to one or more reservoirs of a fluidic system via the microchannel.

In some embodiments, the fluidic system comprises one or more additional components, such as a pressure source (for example, a pump), a detection tool (e.g., a sensor that may be used to detect fluorescence, luminescence, and/or colorimetric changes resulting from activity of a target substrate); and/or a waste stream.

The following examples are intended to illustrate certain embodiments of the present disclosure, but do not exemplify the full scope of the disclosure.

### Example 1

This example describes the preparation and characterization of barcoded nucleic acids, and demonstrates the use of thermal barcodes for determining a target nucleic acid. In this example, genomic DNA was extracted and purified using a Monarch DNA extraction kit (NEB). Nucleic acid targets for DNA detection were PCR amplified with Q5 High-Fidelity 2× master mix (NEB) and purified using KAPA Pure beads (Roche). The melting curves of thermal barcodes (TB) and target regions were simulated using uMelt BatchSM. TBs were designed with expected peak melting temperatures of between 55° C. and 90° C. and lengths of 20-55 nucleotides. Primers for target regions were designed using NCBI Primer-BLAST using default parameters, assuming a target nucleic acid size of 100-140 nucleotides and primer size of 18-20 nucleotides. Optical signaling entities comprising a terminal fluorophore and a terminal quencher were configured to bind to the target nucleic acid, and had a size between 30-40 nucleotides. Primers with TBs and signaling entities were ordered as DNA (Integrated DNA Technologies). All primers, signaling entities, and TBs used in the study are available in Tables 2-6.

TABLE 2
Sequences of target genes that comprised target nucleic acids.
Target regions are type-set in boldface, and sequence
portions complementary to primers for the
target region are underlined.

| | SEQ. | |
|---|---|---|
| Name | ID. NO. | Sequence |
| 3T3 cell | 1 | CACGGTAATGTTATGGTGGGTTTCTTGCTTTCAGCCTCGCTTTTATA |
| Fabp4 gene | | AACTTCCCCTAACGTTTTACTGCTCGAATTTACTCCACATGCACTAC |
| template | | TGTATGACTCATAAAACATGCTAATCATTGTGAATATCGGGACAGC |
| (990 base | | TTAAGAGCAAGAGGAAATGCGCCCTCTGCGCACTGACGGAGCTGG |
| pairs) | | GTTAGGTATGGGAGCTCCGAGGCTGTCAGTCCACCAAGGGAAGCT |
| | | TTTGTCCTTCCCCTTGAGCTGAAGTCGCTTCCTGCAGGCCGTG |
| | | GAATAGAAGCCTGGCCCCCCTCACTCTGCATCAGCTCCTTGTT |
| | | CCTCACCTTCACCTTCCTGTCGTCTGCGGTGATTTCATCGAATTCC |
| | | ACGCCCAGTTTGAAGGAAATCTCGGTGTTTTTAAAAGTACTCTCTG |
| | | ACCGGATGGTGACCAAATCCCCATTTACGCTGATGATCATGTTGGG |
| | | CTTGGCCATGCCTGCCACTTTCCTTGTGGCAAAGCCCACTCCTACA |
| | | GGGAGAAAGAGAACGCATGTAAAGCTAACATTTGTGTGTGTGATG |
| | | AAGAAGACACCAGGGTGCCGGAGGCTCCTTTGATGCTCTCAGAAC |
| | | CTGTCATCCATCCAGATTTACTGTGCCTGTCTGCTAACTACACTCCT |
| | | TCAGATAAGTCCTTAATTTCAACAAGAATGTATTAAGAGATTAAAT |
| | | CTTCATATCTCGATGTCTATGTGTTTTATATAGGTCTGTATGTCTAT |
| | | ATTTATTATAATATGCTAATAATAAGTGTATGTATATTATCTCTATT |
| | | TTCTATTTGTGATTGAGGCAGTTTGACCATTTTATTCTTTCTTAATT |
| | | ACTAAAAGTTTTTTTTTTTTTTAAAATCTAAGTCAAAGGCCAGGCCT |
| | | CACTTAGAGTTATTGTGTTAAAAGGTCATCTATTTTTGAGTGAACA |
| | | AAAATCACATTTCACAACACTTGTTGCCAAATTTAATTTTCTAGGCT |
| | | TCTTTCCCATATTACTCCCTCATGAA |
| Covid-19 | 2 | ATGTCTGATAATGGACCCCAAAATCAGCGAAATGCACCCCGCATTA |
| N gene | | CGTTTGGTGGACCCTCAGATTCAACTGGCAGTAACCAGAATGGAG |
| template | | AACGCAGTGGGGCGCGATCAAAACAACGTCGGCCCCAAGGTTTAC |
| (1260 base | | CCAATAATACTGCGTCTTGGTTCACCGCTCTCACTCAACATGGCAA |
| pairs) | | GGAAGACCTTAAATTCCCTCGAGGACAAGGCGTTCCAATTAACACC |
| | | AATAGCAGTCCAGATGACCAAATTGGCTACTACCGAAGAGCTACC |
| | | AGACGAATTCGTGGTGGTGACGGTAAAATGAAAGATCTCAGTCCA |
| | | AGATGGTATTTCTACTACCTAGGAACTGGGCCAGAAGCTGGACTTC |
| | | CCTATGGTGCTAACAAAGACGGCATCATATGGGTTGCAACTGA |
| | | GGGAGCCTTGAATACACCAAAAGATCACATTGGCACCCGCAAT |
| | | CCTGCTAACAATGCTGCAATCGTGCTACAACTTCCTCAAGGAA |
| | | CAACATTGCCAAAAGGCTTCTACGCAGAAGGGAGCAGAGGCGG |
| | | CAGTCAAGCCTCTTCTCGTTCCTCATCACGTAGTCGCAACAGTTCA |
| | | AGAAATTCAACTCCAGGCAGCAGTAGGGGAACTTCTCCTGCTAGA |
| | | ATGGCTGGCAATGGCGGTGATGCTGCTCTTGCTTTGCTGCTGCTTG |
| | | ACAGATTGAACCAGCTTGAGAGCAAAATGTCTGGTAAAGGCCAAC |
| | | AACAACAAGGCCAAACTGTCACTAAGAAATCTGCTGCTGAGGCTT |
| | | CTAAGAAGCCTCGGCAAAAACGTACTGCCACTAAAGCATACAATG |
| | | TAACACAAGCTTTCGGCAGACGTGGTCCAGAACAAACCCAAGGAA |
| | | ATTTTGGGGACCAGGAACTAATCAGACAAGGAACTGATTACAAAC |
| | | ATTGGCCGCAAATTGCACAATTTGCCCCCAGCGCTTCAGCGTTCTT |
| | | CGGAATGTCGCGCATTGGCATGGAAGTCACACCTTCGGGAACGTG |
| | | GTTGACCTACACAGGTGCCATCAAATTGGATGACAAAGATCCAAA |
| | | TTTCAAAGATCAAGTCATTTTGCTGAATAAGCATATTGACGCATAC |
| | | AAAACATTCCCACCAACAGAGCCTAAAAAGGACAAAAAGAAGAA |
| | | GGCTGATGAAACTCAAGCCTTACCGCAGAGACAGAAGAAACAGCA |
| | | AACTGTGACTCTTCTTCCTGCTGCAGATTTGGATGATTTCTCCAAAC |
| | | AATTGCAACAATCCATGAGCAGTGCTGACTCAACTCAGGCCTAA |

TABLE 3
Sequences of primers for target genes.

| | SEQ. | |
|---|---|---|
| | ID. | |
| Name | NO. | Sequence |
| Forward Primer-Fabp4 | 3 | CACGGTAATGTTATG |
| gene | | GTGGGTTTCTTGCTT |
| Reverse Primer-Fabp4 | 4 | TTCATGAGGGAGTAA |
| | | TATGGGAAAGAAGCC |
| gene | | TAG |

TABLE 4
Sequences of primers for target nucleic
acids (sub-portions of target genes),
designed using NCBI Primer-BLAST.

| | SEQ. | |
|---|---|---|
| | ID. | |
| Name | NO. | Sequence |
| Forward Primer- | 5 | TTGTCCTTCC |
| Fabp4 gene-TR | | CCTTGAGCTG |
| Reverse Primer- | 6 | CAGGAAGGTG |
| Fabp4 gene-TR | | AAGGTGAGGA |
| Forward Primer- | 7 | GTGCTAACAA |
| N gene-TR | | AGACGGCA |
| Reverse Primer- | 8 | GCGTAGAAGC |
| N gene-TR | | CTTTTGGC |

TABLE 5
Sequences of optical signaling entities
for target nucleic acids, including a
5'Hex fluorophore or a 5'Cy5 fluorophore
and a 3' Iowa Black fluorescent quencher
(31ABKFQ), a TAO Quencher, and/or a 3'
Iowa Black red fluorescent quencher
(3IAbRQSp).

| | SEQ. | |
|---|---|---|
| Name | ID. NO. | Sequence |
| HEX- | 9 | /5HEX/CCGGCTTGTCCTTCCCCTT |
| Fabp4 | | GAGCTGAAGTCGCCGG/3IABKFQ/ |
| Cy5- | 10 | /5Cy5/TTGTCCTTC/TAO/CCCTT |
| Fabp4 | | GAGCTGAAGTC/31AbRQSp/ |

TABLE 6
Sequences of thermal barcodes used for forward or reverse primers.

| | SEQ. ID. | | | SEQ. ID. | |
|---|---|---|---|---|---|
| Name | NO. | Forward | Name | NO. | Reverse |
| TB1 | 11 | GGGTTTTTTTTTTTTT | TB11 | 21 | CCCCCTTTTTTTTTTTTT |
| | | TTCCTTTTTTTTTTTC | | | TTTTTTTTTTTTTTTTTT |
| | | CTTTTTTT | | | TTTTT |
| TB2 | 12 | GGGTTTTTCCCTTTT | TB12 | 22 | CCCCCTTTTTTTTTTTTT |
| | | TTTCCTTTTTTTTTTT | | | TTTTCCCTTTTTTTTTTT |
| | | CCTTTTTTTTT | | | TTTTT |
| TB3 | 13 | GGGTTTTTCCCTTTT | TB13 | 23 | CCCCCTTTTTTCCTTTTT |
| | | TTTCCTTTTCCTTTTT | | | TTTTCCCTTTTTTTTTTT |
| | | CCTTTTTTT | | | TTTTT |
| TB4 | 14 | GGGTTTTTCCCTTTC | TB14 | 24 | CCCCCTTTTTTCCTTCTT |
| | | TTTCCTTTTCCTTTTT | | | CTTTCCCTTTTTTTTTTT |
| | | CCTTTTTTT | | | TTTTT |
| TB5 | 15 | GGGTTTTTCCCTTTC | TB15 | 25 | CCCCCTTCTTTCCTTCTT |
| | | TTTCCTTTTCCTTTTT | | | CTTTCCCTTTTTTTTTTT |
| | | CCTTCCTTT | | | CTTTT |
| TB6 | 16 | GGGTTTTTTCTTTTTT | TB16 | 26 | CCCCCTTTTTTTTTTTTT |
| | | TTCCTTTTTTTTTTTC | | | TCTTTTTTTTTTTTCTTT |
| | | CTTTTTTT | | | TTTTT |
| TB7 | 17 | GGGTTCTTCCCTTTC | TB17 | 27 | CCCCCTTCTTTCCTTCTT |
| | | TTTCCTTTTCCTTTTT | | | CTTTCCCTTTTTTTTTTT |
| | | CCTTCCTTT | | | CTGGT |
| TB8 | 18 | GAGTTTTTTTTTCTTT | TB18 | 28 | TCACCTTTTTTTTTTTTT |
| | | TTTCTTTTTTTTCTTC | | | TTTTTTTTTTTTTTTTTT |
| | | TTTTTTTT | | | TTTTC |
| TB9 | 19 | GAGTTTTTCTTTTTT | TB19 | 29 | TCACCTTTTTTTTTTTTT |
| | | CTTTTTTTTCTTCTTT | | | TTTTTTTTTTTTTTTTTC |
| | | TTTTT | | | |
| TB10 | 20 | GAGTTTTTCTTTTAT | TB20 | 30 | TTCACCTTTTTTTTTTAT |
| | | TTAAAAATTTTCTTA | | | ATATTTTTTTTTTTTTTT |
| | | TTTTAAAATT | | | TTTC |

TABLE 6
Sequences of barcoded primers for target nucleic acids above. These
sequences are concatenations of thermal barcode sequences provided
in Table 6 and target nucleic acid primers provided in Table 4.

| | SEQ. | Forward primer sequence | | SEQ. | Reverse primer sequence |
|---|---|---|---|---|---|
| Name | ID. NO. | with Forward-TB | Name | ID. NO. | with Reverse-TB |
| TB1- | 31 | GGGTTTTTTTTTTTTTTT | TB11- | 51 | CCCCCTTTTTTTTTTTTT |
| Fabp4 | | CCTTTTTTTTTTTCCTTT | Fabp4 | | TTTTTTTTTTTTTTTTTT |
| | | TTTTTTGTCCTTCCCCTT | | | TTTTTCAGGAAGGTGA |
| | | GAGCTG | | | AGGTGAGG |
| TB2- | 32 | GGGTTTTTCCCTTTTTTT | TB12- | 52 | CCCCCTTTTTTTTTTTTT |
| Fabp4 | | CCTTTTTTTTTTTCCTTT | Fabp4 | | TTTTCCCTTTTTTTTTTT |
| | | TTTTTTGTCCTTCCCCTT | | | TTTTTCAGGAAGGTGA |
| | | GAGCTG | | | AGGTGAGG |
| TB3- | 33 | GGGTTTTTCCCTTTTTTT | TB13- | 53 | CCCCCTTTTTTCCTTTTT |
| Fabp4 | | CCTTTTCCTTTTTCCTTT | Fabp4 | | TTTTCCCTTTTTTTTTTT |
| | | TTTTTTGTCCTTCCCCTT | | | TTTTTCAGGAAGGTGA |
| | | GAGCTG | | | AGGTGAGG |
| TB4- | 34 | GGGTTTTTCCCTTTCTT | TB14- | 54 | CCCCCTTTTTTCCTTCTT |
| Fabp4 | | TCCTTTTCCTTTTTCCTT | Fabp4 | | CTTTCCCTTTTTTTTTTT |
| | | TTTTTTTGTCCTTCCCCT | | | TTTTTCAGGAAGGTGA |
| | | TGAGCTG | | | AGGTGAGG |
| TB5- | 35 | GGGTTTTTCCCTTTCTT | TB15- | 55 | CCCCCTTCTTTCCTTCTT |
| Fabp4 | | TCCTTTTCCTTTTTCCTT | Fabp4 | | CTTTCCCTTTTTTTTTTT |
| | | CCTTTTTGTCCTTCCCC | | | CTTTTCAGGAAGGTGA |
| | | TTGAGCTG | | | AGGTGAGG |
| TB6- | 36 | GGGTTTTTTCTTTTTTTT | TB16- | 56 | CCCCCTTTTTTTTTTTTT |
| Fabp4 | | CCTTTTTTTTTTTCCTTT | Fabp4 | | TCTTTTTTTTTTTTCTTT |
| | | TTTTTTGTCCTTCCCCTT | | | TTTTTCAGGAAGGTGA |
| | | GAGCTG | | | AGGTGAGG |
| TB7- | 37 | GGGTTCTTCCCTTTCTT | TB17- | 57 | CCCCCTTCTTTCCTTCTT |
| Fabp4 | | TCCTTTTCCTTTTTCCTT | Fabp4 | | CTTTCCCTTTTTTTTTTT |
| | | CCTTTTTGTCCTTCCCC | | | CTGGTCAGGAAGGTGA |
| | | TTGAGCTG | | | AGGTGAGG |
| TB8- | 38 | GAGTTTTTTTTTCTTTTT | TB18- | 58 | TCACCTTTTTTTTTTTTT |
| Fabp4 | | TCTTTTTTTTCTTCTTTT | Fabp4 | | TTTTTTTTTTTTTTTTTT |
| | | TTTTTTGTCCTTCCCCTT | | | TTTTCCAGGAAGGTGA |
| | | GAGCTG | | | AGGTGAGG |
| TB9- | 39 | GAGTTTTTCTTTTTTCTT | TB19- | 59 | TCACCTTTTTTTTTTTTT |
| Fabp4 | | TTTTTTCTTCTTTTTTTT | Fabp4 | | TTTTTTTTTTTTTTTTTC |
| | | TTGTCCTTCCCCTTGAG | | | CAGGAAGGTGAAGGTG |
| | | CTG | | | AGGA |
| TB10- | 40 | GAGTTTTTCTTTTATTT | TB20- | 60 | TTCACCTTTTTTTTTTAT |
| Fabp4 | | AAAAATTTTCTTATTTT | Fabp4 | | ATATTTTTTTTTTTTTTT |
| | | AAAATTTTGTCCTTCCC | | | TTTCCAGGAAGGTGAA |
| | | CTTGAGCTG | | | GGTGAGGA |
| TB1-N | 41 | GGGTTTTTTTTTTTTTTT | TB11-N | 61 | CCCCCTTTTTTTTTTTTT |
| gene | | CCTTTTTTTTTTTCCTTT | gene | | TTTTTTTTTTTTTTTTTT |
| | | TTTTGTGCTAACAAAGA | | | TTTTTGCGTAGAAGCCT |
| | | CGGCA | | | TTTGGC |
| TB2-N | 42 | GGGTTTTTCCCTTTTTTT | TB12-N | 62 | CCCCCTTTTTTTTTTTTT |
| gene | | CCTTTTTTTTTTTCCTTT | gene | | TTTTCCCTTTTTTTTTTT |
| | | TTTTTTGTGCTAACAAA | | | TTTTTGCGTAGAAGCCT |
| | | GACGGCA | | | TTTGGC |
| TB3-N | 43 | GGGTTTTTCCCTTTTTTT | TB13-N | 63 | CCCCCTTTTTTCCTTTTT |
| gene | | CCTTTTCCTTTTTCCTTT | gene | | TTTTCCCTTTTTTTTTTT |
| | | TTTTGTGCTAACAAAGA | | | TTTTTGCGTAGAAGCCT |
| | | CGGCA | | | TTTGGC |
| TB4-N | 44 | GGGTTTTTCCCTTTCTT | TB14-N | 64 | CCCCCTTTTTTCCTTCTT |
| gene | | TCCTTTTCCTTTTTCCTT | gene | | CTTTCCCTTTTTTTTTTT |
| | | TTTTTGTGCTAACAAAG | | | TTTTTGCGTAGAAGCCT |
| | | ACGGCA | | | TTTGGC |
| TB5-N | 45 | GGGTTTTTCCCTTTCTT | TB15-N | 65 | CCCCCTTCTTTCCTTCTT |
| gene | | TCCTTTTCCTTTTTCCTT | gene | | CTTTCCCTTTTTTTTTTT |
| | | CCTTTGTGCTAACAAAG | | | CTTTTGCGTAGAAGCCT |
| | | ACGGCA | | | TTTGGC |
| TB6-N | 46 | GGGTTTTTTCTTTTTTTT | TB16-N | 66 | CCCCCTTTTTTTTTTTTT |
| gene | | CCTTTTTTTTTTTCCTTT | gene | | TCTTTTTTTTTTTTCTTT |
| | | TTTTGTGCTAACAAAGA | | | TTTTTGCGTAGAAGCCT |
| | | CGGCA | | | TTTGGC |
| TB7-N | 47 | GGGTTCTTCCCTTTCTT | TB17-N | 67 | CCCCCTTCTTTCCTTCTT |
| gene | | TCCTTTTCCTTTTTCCTT | gene | | CTTTCCCTTTTTTTTTTT |
| | | CCTTTGTGCTAACAAAG | | | CTGGTGCGTAGAAGCC |
| | | ACGGCA | | | TTTTGGC |
| TB8-N | 48 | GAGTTTTTTTTTCTTTTT | TB18-N | 68 | TCACCTTTTTTTTTTTTT |
| gene | | TCTTTTTTTTCTTCTTTT | gene | | TTTTTTTTTTTTTTTTTT |
| | | TTTTGTGCTAACAAAGA | | | TTTTCGCGTAGAAGCCT |
| | | CGGCA | | | TTTGGC |
| TB9-N | 49 | GAGTTTTTCTTTTTTCTT | TB19-N | 69 | TCACCTTTTTTTTTTTTT |
| gene | | TTTTTTCTTCTTTTTTTT | gene | | TTTTTTTTTTTTTTTTTC |
| | | GTGCTAACAAAGACGG | | | GCGTAGAAGCCTTTTG |
| | | CA | | | GC |
| TB10-N | 50 | GAGTTTTTCTTTTATTT | TB20-N | 70 | TTCACCTTTTTTTTTTAT |
| gene | | AAAAATTTTCTTATTTT | gene | | ATATTTTTTTTTTTTTTT |
| | | AAAATTGTGCTAACAA | | | TTTCGCGTAGAAGCCTT |
| | | AGACGGCA | | | TTGGC |

To determine the melting profiles of the thermal barcodes, optical signaling entities, and targeted regions in a bulk liquid, qPCR was used in 1× Q5 High-Fidelity master mix (NEB), 2× Evagreen plus dye (Biotium), 500 nM barcoded forward primer, 500 nM barcoded reverse primer, 250 nM optical signaling entity, 15.25 microliters of ddH₂O, and 1 microliter of the target gene comprising the target nucleic acid were mixed in a 96-well PCR plate to form a reaction mixture. PCR and melting profile analysis were performed on a qPCR platform (Bio-Rad CFX96 Touch Real-Time PCR Detection System).

The PCR thermal conditions were: 1 cycle of 95° C. for 3 min; 35 cycles of 95° C. for 30 s, 55° C. for 10 s and 72° C. for 10 s; and 1 cycle of 72° C. 5 min and 12° C. 30 s. Fluorescence was monitored during cycling. To measure the melting profiles, the reaction mixture was heated gradually from 55 to 95° C. at a rate of 0.2 to 1° C./s, holding each temperature for 0.05 s, with continuous monitoring of the fluorescence of the system.

To determine the melting profiles of barcoded nucleic acids, the reaction mixture was partitioned into nanoliter-sized droplets using microfluidic chips or using a QX200 Droplet Generator (Bio-Rad). Emulsions comprising the droplets were transferred into a 96 well PCR plate. After PCR was performed, melting profiles were extracted by adding Eva Green Dye as a non-specific binding detection agent and continuously monitoring the fluorescence of the binding detection agent with the increasing temperature on the qPCR platform (Bio-Rad CFX96 Touch Real-Time PCR Detection System). Melting curve analysis was performed by using uAnalyze℠ to process the measured intensity of the binding detection agent, and to differentiate the measured integrated melting profile.

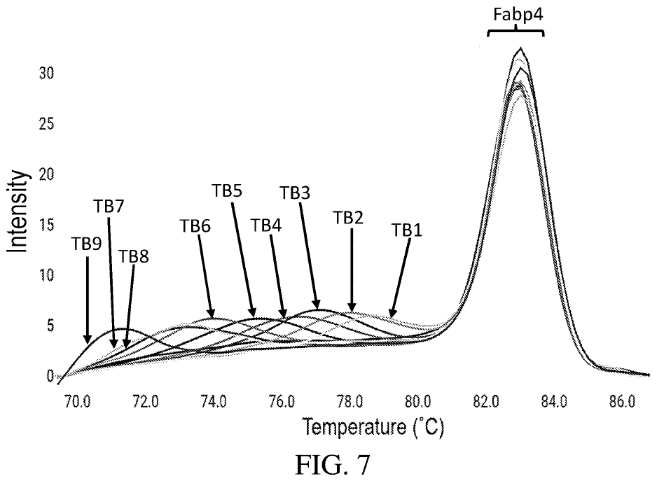

FIG. 7 presents the melting profiles of barcoded target nucleic acids amplified from the Fabp4 gene. FIG. 7 includes 9 overlaid melting profiles, representing the melting profile of each barcoded target nucleic acid. The curves are marked by arrows that indicate the thermal barcode used in each barcoding step. The common peaks of the curves, labeled collectively “Fabp4”, indicate the portion of the melting profile that results from melting of the target nucleic acid from the Fabp4 gene. As shown, the thermal barcodes TB1-TB9 have well-separated melting profiles, and the barcode had little to no impact on the melting profile of the target nucleic acid.

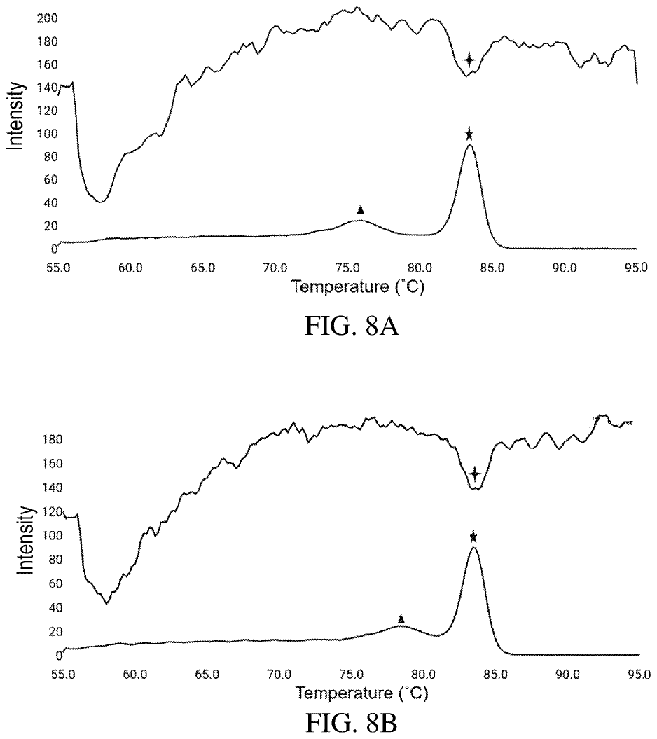

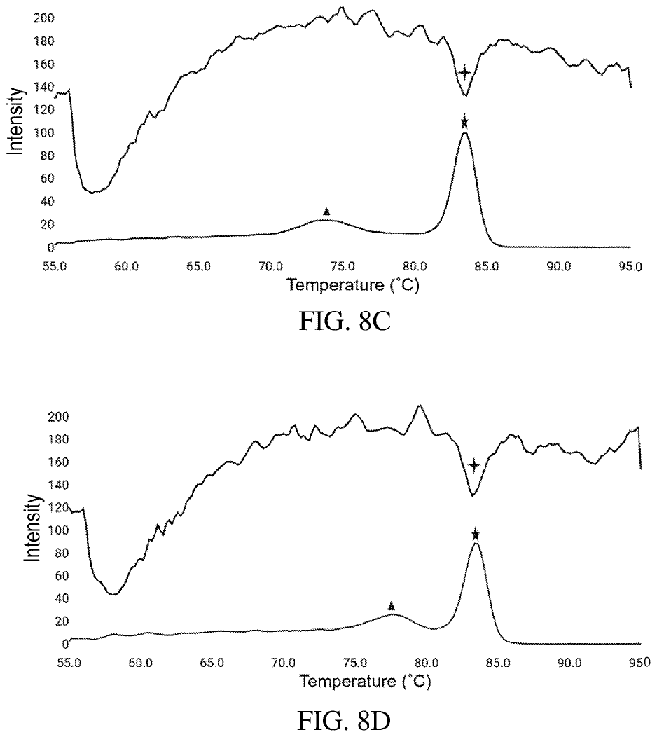

FIGS. 8A-8D present the melting profiles of the target Fabp4 nucleic acid, barcoded with TB1-TB4, respectively, in separate graphs. These melting profiles are the same as those shown in FIG. 7. In each of FIGS. 8A-8D, a black triangle denotes a peak melting temperature corresponding to the thermal barcode and a black five-pointed star denotes a peak melting temperature corresponding to the melting of the target nucleic acid. Additionally, FIGS. 8A-8D overlay the derivative of fluorescent signal detected from the optical signaling entity. The valley within the fluorescence derivative, indicated by the four-pointed star, corresponds to a decrease in fluorescence resulting from the melting of the target nucleic acid, which results from the melting of the optical signaling entity away from the target nucleic acid. Once the optical signaling entity was released by the melting process, the quencher had a closer average proximity to the fluorophore, decreasing the fluorescence of the optical signaling entity. This explains why the peak melting temperature of the target nucleic acid (indicated by the five-pointed star) corresponds so closely to the point at which the fluorescence changes most rapidly, indicated by the four-pointed star, in FIGS. 8A-8D.

These examples demonstrate that the melting profile of thermally barcoded target nucleic acids can be established, and can be used to distinguish between the thermally barcoded nucleic acids. Furthermore, they demonstrate that optical signaling entities may be used as a secondary detectors, and they demonstrate the overall viability of the methods detailed herein.

### Example 2

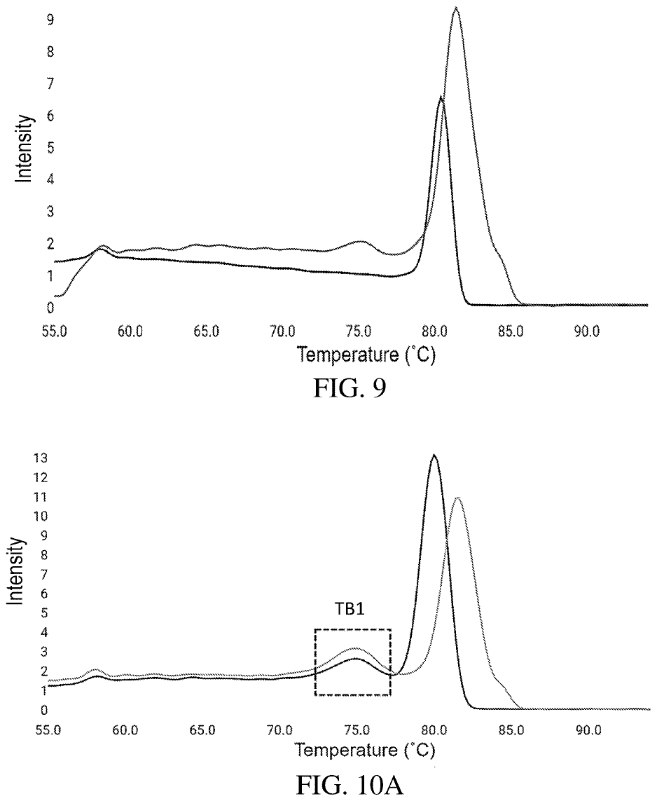

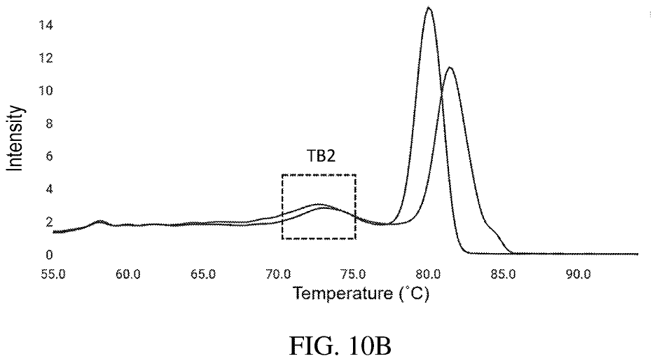

In this example, the target nucleic acid of the Fabp4 gene and the target nucleic acid of the COVID-19 N-gene (described in Table 2 of Example 1) were compared with and without barcoding. The appropriate protocols and primers of Example 1 were used. The melting profiles of the target nucleic acids without thermal barcodes are presented in FIG. 9. As shown, the melting profiles of the Fabp4 target nucleic acid was distinguishable from the melting profile of the COVID-19 N-gene, even without barcoding. FIGS. 10A-10B, meanwhile, show the melting profile of the barcoded target nucleic acids. FIG. 10A presents the melting profile of the target nucleic acids, barcoded with TB1. FIG. 10B presents the melting profile of the target nucleic acids, barcoded with TB2. As shown, the barcodes had approximately similar melting profiles in both cases. Furthermore, the melting profiles of the target nucleic acids themselves was unaffected by barcoding, as can be seen by comparing FIGS. 10A-10B with FIG. 9. The change in relative heights of the peaks is an arbitrary artifact of the labeling and measurement process—the shape and temperature range of the melting profile of the target nucleic acids did not change as a result of amplification using barcoded primers.

This example demonstrates that barcoding is a reliable method for modifying nucleic acids, and that thermal barcodes can be used as a reliable index for multiplex determination of target nucleic acids. Furthermore, this example demonstrates that, as shown in FIGS. 10A-10B, a thermal barcode may be used multiple times to label target nucleic acids with non-overlapping melting profiles. Thus, while thermal barcodes can be used to distinguish target nucleic acids with closely overlapping melting peaks, it is the overall uniqueness of the melting profile of the barcoded nucleic acid that determines how many target nucleic acids may simultaneously be processed using a plurality of primers.

### Example 3

This example demonstrates the use of thermal barcoding to determine a target nucleic acid in a plurality of droplets, rather than within a bulk solution.

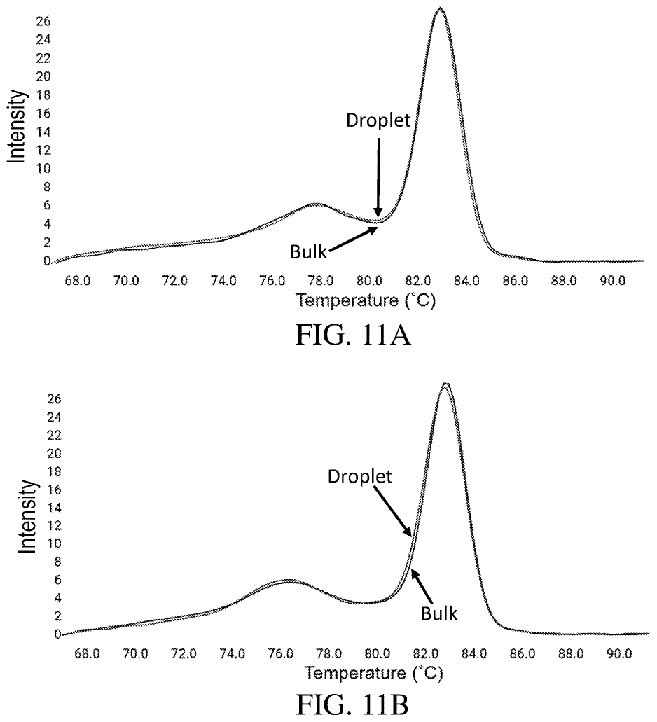

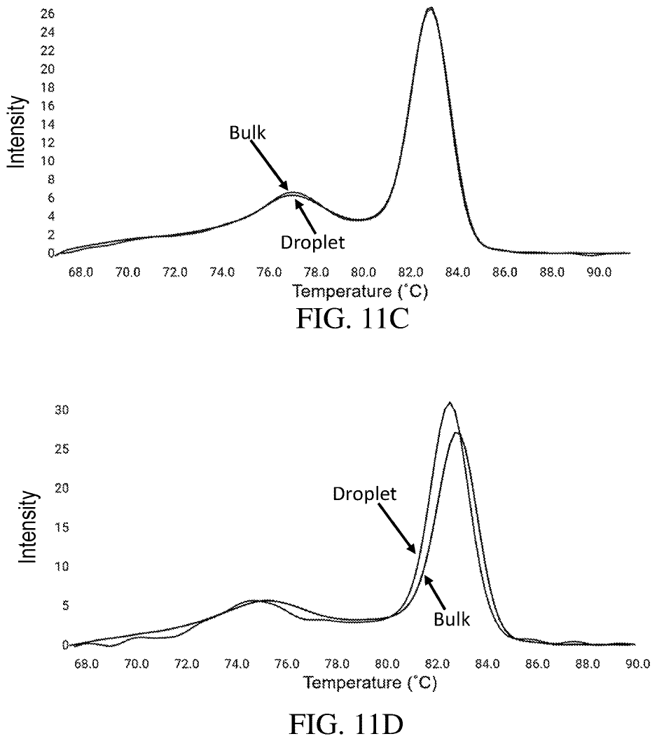

To determine the melting profiles of each droplet, reaction mixtures described in Example 1 above were partitioned into nanoliter-sized droplets. Droplets were generated using a microfluidic chip or QX200 Droplet Generator (Bio-Rad). After PCR on a thermal cycler, droplets were loaded into a microfluidic detection chip. Images of the droplets were acquired with increasing temperature at different color channels using a commercial microscope (e.g., AxioZoom V16 Microscope) or a homemade camera. The fluorescent intensity of each droplet over temperature was measured by ImageJ. The melting curves of each droplet were analyzed by uAnalyze℠. FIGS. 11A-11D present the results, comparing the melting profiles observed in droplets to the melting profiles reported in Example 1 for TB1-TB4, respectively. As indicated, the droplet-based method agrees with the bulk method regarding the melting profile of both the target nucleic acid and the thermal barcode in every case. This example demonstrates the viability of nanoscale measurement of droplet melting profiles, and thus demonstrates the viability of droplet-based methods of target nucleic acid determination.

### Example 4

This example describes the preparation and characterization of a plurality of primers suitable for multiplex determination of nucleic acids. The primers comprised either a first thermal barcode T1 (low-melting) or a second thermal barcode T2 (high-melting). Each thermal barcode included between 15 and 20 base pairs. The primers further comprised a hairpin region of about 10 base pairs comprising either a first optical signaling entity comprising a 5′Hex fluorophore and a quencher (the first optical signaling entity is referred to hereafter as the “Hex probe”), or a second optical signaling entity comprising a ROX (carboxy-X-rhodamine) fluorophore and a quencher (the second optical signaling entity is referred to hereafter as the “ROX probe”). When used together to amplify a nucleic acid strand to be determined, the amplified strand comprised both the first and the second thermal barcodes and both the first and the second optical signaling entities, resulting in an amplified strand comprising four uniquely identifiable melting transitions.

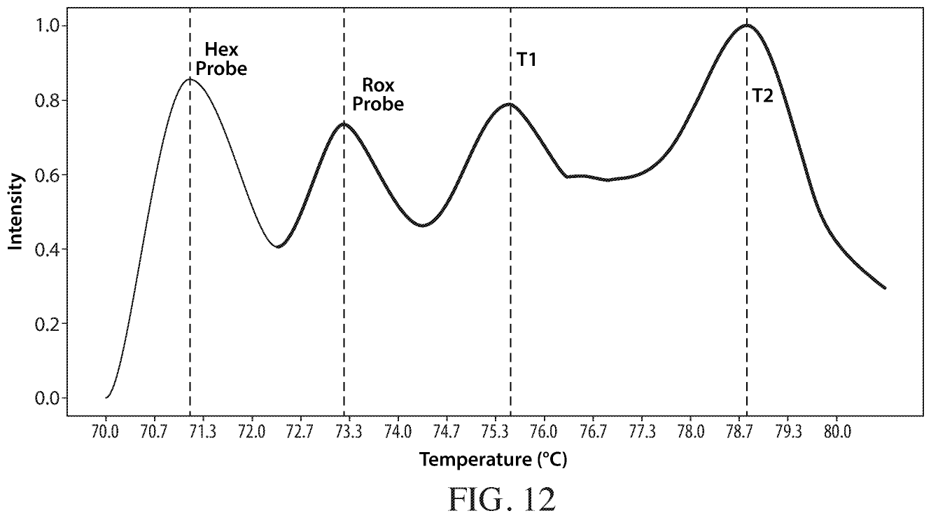

FIG. 12 presents the melting transitions associated with an exemplary pair of primers, illustrating the four distinct melting peaks useful for multiplex detection. This example demonstrates the viability of multiplex detection. By altering the sequences of both the optical signaling moieties and the thermal barcodes to change their melting profiles, a diverse plurality of primers may be produced (e.g., in one or more droplets) and used to label amplified nucleic acids using their unique melting temperature patterns.

### Example 5

This example describes the preparation and characterization of target nucleic acids amplified using a plurality of 60 unique primers suitable for multiplex determination of nucleic acids. The primers comprised either (i) an optical signaling moiety comprising a ROX fluorophore and quencher; or (ii) an optical signaling moiety comprising a Hex fluorophore and quencher, as previously described. To perform these measurements, a dPCR chip available from Thermo-Fisher was used to separate barcodes into 20,000 wells, each having a diameter of 50 μm and a depth of 370 μm for a total volume of 725 μL per well.

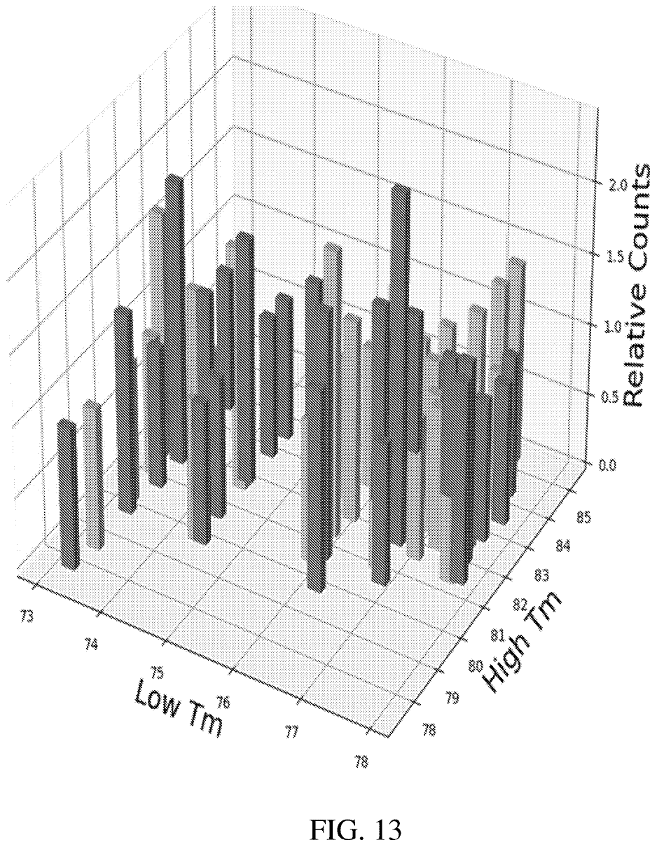

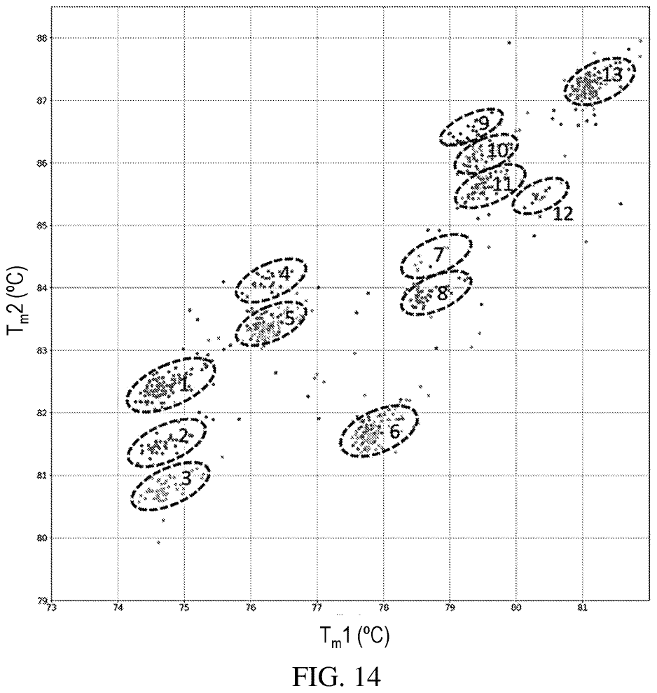

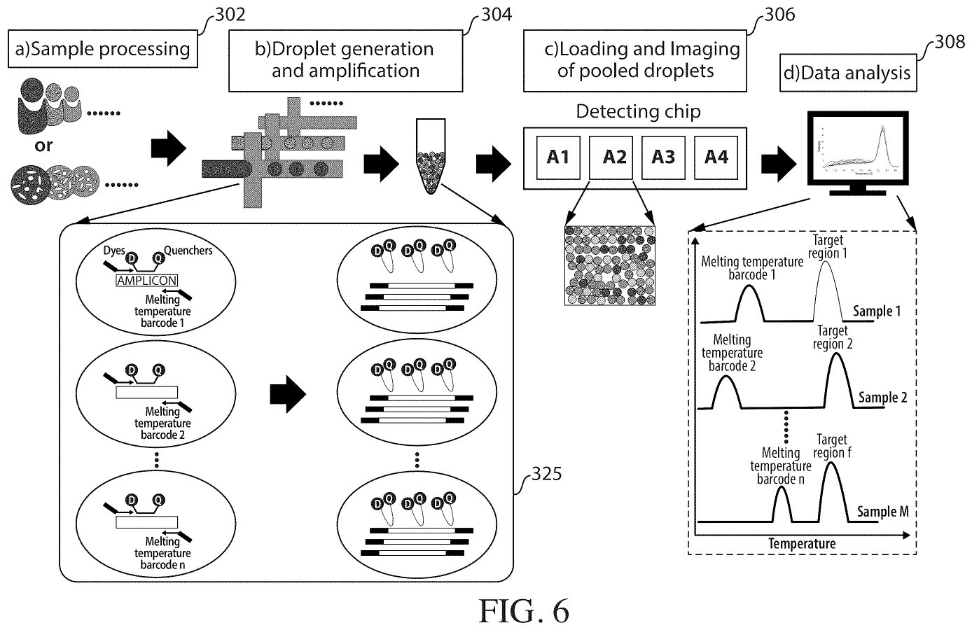

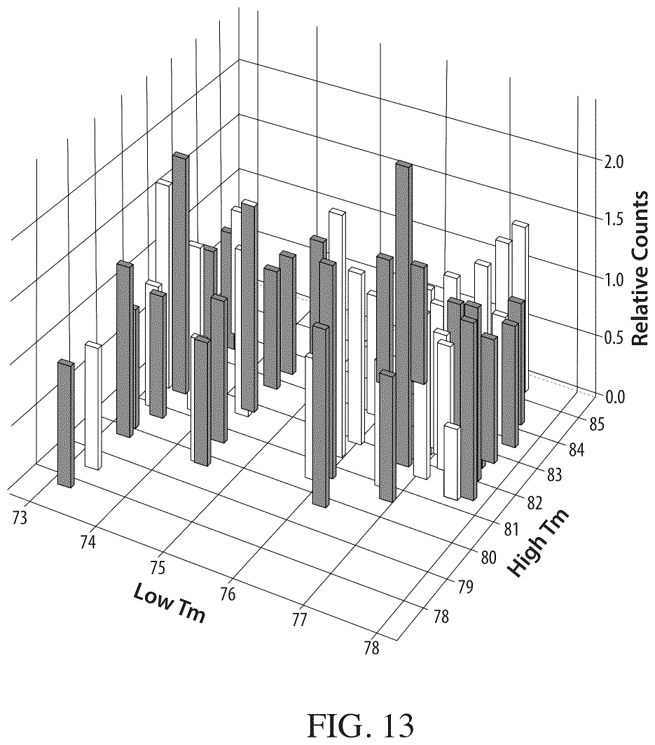

Fluorescence imaging of each well indicated that the primers were typically separated into individual wells, and that the presence or absence of optical signaling moieties could be uniquely detected. FIG. 13 shows the distribution of relative counts of barcodes at various melting temperatures (Tₘ) associated with the low-melting thermal barcode (low Tₘ) and the high-melting thermal barcode (high Tₘ) measured for the barcodes. The bars in the histogram are color-coded based on the type of optical signaling moiety used. The highly discretized distribution clearly illustrates how the primers may be used to determine and distinguish the amplified target nucleic acids in each well—the distinct melting profiles and optical signals associated with each nucleic acid resulted in clearly distinct count distributions.

### Example 6

This example describes the determination of a plurality of genetic variations crucial for cancer diagnostics using a plurality of nucleic acids comprising thermal barcodes. To identify these variations, a ligation-based method was used to join the nucleic acids comprising thermal barcodes to target nucleic acids comprising specific DNA segments. Each segment was marked with two unique tags: a left half tag (LHT) and a right half tag (RHT), which were designed for hybridization with universal primers and both of which included a thermal barcode suitable for identifying one of the target nucleic acids. Only perfectly matched tags could be covalently linked by DNA ligase, ensuring specificity. Subsequently, the universal primers were used to amplify the barcoded nucleic acids.

Each amplified sequence included either a first thermal barcode having a first melting temperature (Tₘ1) and a second thermal barcode having a second melting temperature (Tₘ2), such that the amplified gene comprised both the first and the second thermal barcode. Additionally, one of the ligation tags associated with each gene comprised an optical signaling entity, which was used to provide an additional verification of amplification of the target gene. The optical signaling entities comprised either an EvaGreen fluorophore, a Hex fluorophore, or a ROX fluorophore, along with a quencher, as previously described.

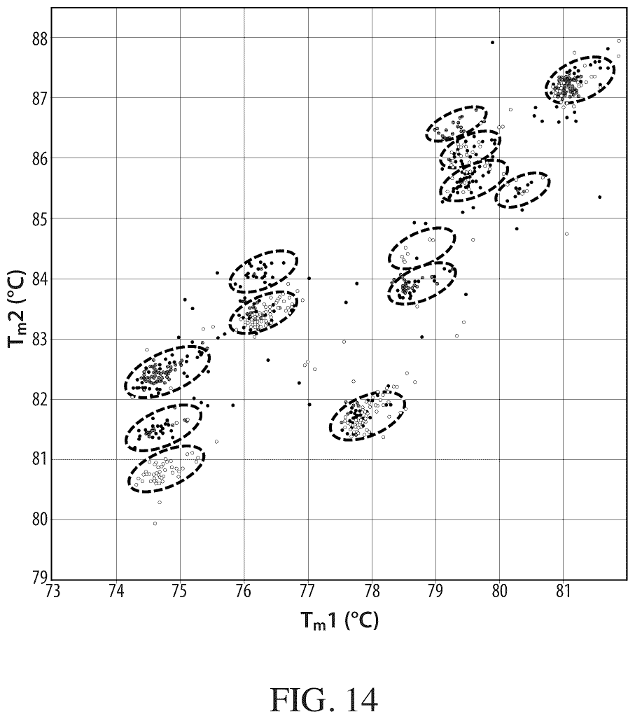

TABLE 7
Clusters identified in FIG. 14 and associated genes
and optical signaling entities identified.

| | | Signaling Moiety/ |
|---|---|---|
| Cluster | Gene | Moieties Present |
| 1 | PIK3CA-E545A | ROX |
| 2 | PIK3CA-ex9 | ROX |
| 3 | PIK3CA-E545K | Hex |
| 4 | PIK3CA-H1047R | ROX |
| 5 | PIK3CA-ex20 | ROX |
| 5 | PIK3CA-H1047L | Hex |
| 6 | BRAF-ex15 | ROX |
| 6 | BRAF-V600E | Hex |
| 7 | EGFR-delete | Hex (one ROX |
| | | observed) |
| 8 | EGFR-ex19 | ROX |
| 9 | KRAS-G12R | EvaGreen |
| 10 | KRAS-ex2 | ROX |
| 10 | KRAS-G12A | Hex |
| 11 | KRAS-G12D | ROX |
| 11 | KRAS-G12V | HEX |
| 12 | KRAS-G12S | ROX |
| 12 | KRAS-G12C | HEX |
| 13 | EGFR-T790 | ROX |
| 13 | EGFR-T790M | HEX |

The plurality of ligation tags was used to amplify and determine 19 gene variations based on their optical signaling and on the melting temperatures of their barcodes. FIG. 14 presents the results. The melting peak positions Tₘ1 and Tₘ2 as measured for each amplified gene are indicated on the x-axis and y-axis respectively. Thirteen total clusters of Tₘ1 and Tₘ2 were uniquely identified, some including optical signal from multiple genes, and those clusters are labeled in FIG. 14. Table 7 identifies the gene variants associated with each cluster, along with a qualitative indication of the optical signal observed for measurements within each cluster. Qualitatively, each cluster matched the predicted optical profile except as indicated in Table 7. Where multiple optical signals were present in each cluster, multiple genes were expected and identified by their ligation tags, as indicated in Table 7, where multiple clusters appear twice. This demonstrates how different optical probes may be used to distinguish between target nucleic acids with identical thermal barcodes. Although FIG. 14 includes a few stray/unidentified measurements, the vast majority of measurements fell within the identified clusters, allowing each gene variant to be uniquely detected. This result demonstrates the unique advantages associated with the methods provided herein.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
amplifying a target nucleic acid using a barcoded primer comprising a primer sequence and a thermal barcode sequence to produce a barcoded nucleic acid, the barcoded nucleic acid comprising a sequence identical to the thermal barcode sequence and a sequence identical to the target nucleic acid,
wherein the thermal barcode sequence exhibits a first peak melting point and the target nucleic acid exhibits a second peak melting point, the first peak melting point and the second peak melting point differing by at least 0.1° C.

2. The method of claim 1, further comprising determining the barcoded nucleic acid by determining the first peak melting point and the second peak melting point.

3. A method, comprising:
amplifying a target nucleic acid using a barcoded primer comprising a primer sequence and a thermal barcode sequence to produce a barcoded nucleic acid, the barcoded nucleic acid comprising a sequence identical to the thermal barcode sequence and a sequence identical to the target nucleic acid,
wherein the thermal barcode sequence has a first ratio of total nearest-neighbor binding enthalpy to total nearest-neighbor binding entropy and the target nucleic acid has a second ratio of total nearest-neighbor binding enthalpy to total nearest-neighbor binding entropy, wherein the first ratio differs from the second ratio by at least 1% of the second ratio.

4. A method, comprising:
amplifying a target nucleic acid using a barcoded primer comprising a primer sequence and a thermal barcode sequence to produce a barcoded nucleic acid, the barcoded nucleic acid comprising a sequence identical to the thermal barcode sequence and a sequence identical to the target nucleic acid,
wherein the thermal barcode sequence comprises less than or equal to 500 nucleotides and wherein at least 55% of the nucleotides of the thermal barcode sequence are A or T or wherein at least 45% of the nucleotides of the thermal barcode are G or C, and
wherein the thermal barcode sequence exhibits a peak melting temperature first peak melting point and the target nucleic acid exhibits a second peak melting point, the first peak melting point and the second peak melting point differing by at least 0.1° C.

5. A method as in any preceding claim, wherein determining the barcoded nucleic acid comprises determining the presence or the absence of the target nucleic acid within a sample.

6. A method as in any preceding claim, wherein determining the barcoded nucleic acid comprises using one or more auxiliary barcodes configured to bind to at least a portion of the thermal barcode sequence or at least a portion of the target nucleic acid.

7. A method as in any preceding claim, wherein the presence of the barcoded nucleic acid is indicative of a disease or condition.

8. A method as in any preceding claim, wherein the absence of the barcoded nucleic acid is indicative of a disease or condition.

9. A method as in any preceding claim, wherein determining the barcoded nucleic acid comprises determining a subject from which the target nucleic acid originated.

10. A method as in any preceding claim, wherein the target nucleic acid is a first target nucleic acid of a plurality of target nucleic acids.

11. The method of any preceding claim, wherein a compartment comprises the nucleic acid.

12. The method of any preceding claim, wherein a droplet comprises the nucleic acid.

13. The method of any preceding claim, wherein a microwell comprises the nucleic acid.

14. A method as in any preceding claim, wherein the target nucleic acid is amplified in a droplet comprising a plurality of barcoded primers comprising primer sequences and thermal barcode sequences.

15. A method as in any preceding claim, wherein the method comprises amplifying a plurality of target nucleic acids using a plurality of primers comprising a plurality of thermal barcode sequences to produce a plurality of barcoded nucleic acids, and determining at least some of the plurality of barcoded nucleic acids at least in part by measuring a peak melting temperature or a temperature-dependent melting profile of the thermal barcode sequences.

16. A method as in any preceding claim, wherein the method further comprises forming a first plurality of droplets, at least 90% of which contain either only one target nucleic acid or no target nucleic acid, and at least 90% of which contain at least one primer.

17. A method as in any preceding claim, wherein the method further comprises breaking the first plurality of droplets to mix the amplified nucleic acids.

18. A method as in any preceding claim, wherein the method further comprises forming a second plurality of droplets, at least 90% of which contains either one of the amplified nucleic acids or no amplified nucleic acid, and at least 90% of which contain at least one primer.

19. A method as in any preceding claim, wherein determining the nucleic acid further comprises exposing detecting the target nucleic acid and/or the barcoded nucleic acid using an optical signaling entity.

20. A method as in any preceding claim, wherein the optical signaling entity comprises a fluorophore.

21. A method as in any preceding claim, wherein the optical signaling entity comprises a fluorescent quencher.

22. An article, comprising:
a plurality of droplets comprising a first droplet and a second droplet, wherein:
the first droplet comprises a first target nucleic acid and a first barcoded primer comprising a first primer sequence for the first target nucleic acid and a first thermal barcode sequence; and
the second droplet comprises a second target nucleic acid and a barcoded primer comprising a second primer sequence for the second target nucleic acid and a second thermal barcode sequence;
wherein a peak melting temperature of the first thermal barcode sequence differs from a peak melting temperature of the second thermal barcode sequence by at least 0.1° C.

23. An article as in claim 22, wherein the second target nucleic acid is the same as the first target nucleic acid.

24. An article, comprising:
a plurality of droplets comprising a first droplet and a second droplet, wherein:
the first droplet comprises a first target nucleic acid and a first barcoded primer comprising a first primer sequence for the first target nucleic acid and a first thermal barcode sequence; and
the second droplet comprises a second target nucleic acid and a second barcoded primer comprising a second primer sequence for the second target nucleic acid and a second thermal barcode sequence;
wherein the first thermal barcode sequence has a first ratio of total nearest-neighbor binding enthalpy to total nearest-neighbor binding entropy and the second thermal barcode sequence has a second ratio of total nearest-neighbor binding enthalpy to total nearest-neighbor binding entropy, wherein the first ratio differs from the second ratio by at least 1% of the second ratio.

25. A method or article as in any preceding claim, wherein a barcoded primer comprises a plurality of thermal barcode sequences.

26. A method or article as in any preceding claim, wherein the first thermal barcode sequence has a peak melting temperature that differs by at least 1° C. from a peak melting temperature of all other thermal barcode sequences of the plurality of thermal barcode sequences.

27. A method or article as in any preceding claim, wherein the plurality of thermal barcode sequences each have distinct temperature-dependent melting profiles.

28. A method or article as in any preceding claim, wherein the temperature-dependent melting profile of the thermal barcode sequence differs from a temperature-dependent melting profile of the target nucleic acid.

29. A method or article as in any preceding claim, wherein the thermal barcode sequence melts within a first temperature range and the target nucleic acid melts within a second temperature range different from the first temperature range.

30. A method or article as in any preceding claim, wherein the first temperature range is separated from the second temperature range by greater than or equal to 2° C.

31. A method or article as in any preceding claim, wherein the first temperature range is totally below the second temperature range.

32. A method or article as in any preceding claim, wherein the first temperature range is totally above the second temperature range.

33. A method or article as in any preceding claim, wherein the article comprises a first plurality of droplets, at least 90% of which contain either only one target nucleic acid or no target nucleic acid, and at least 90% of which contain at least one primer.

34. A method of determining a target substrate, the method comprising:
exposing a library of barcoded nucleic acids to the target substrate such that at least some of the barcoded nucleic acids are able to bind to the target substrate;
removing barcoded nucleic acids that do not bind to the target substrate; and
identifying at least some of the barcoded nucleic acids that are able to bind to the target substrate using a melting profile of at least one thermal barcode of the barcoded nucleic acids.

35. The method of claim 34, further comprising amplifying the barcoded nucleic acids that are able to bind to the target substrate.

36. The method of any one of claims 34-35, further comprising identifying the at least some of the barcoded nucleic acids that are able to bind to the target substrate using an auxiliary barcode.

37. The method of any one of claims 34-36, wherein the step of identifying at least some of the barcoded nucleic acids comprises identifying a peak melting temperature of at least one barcode of the barcoded nucleic acids.

38. An article, comprising:
a plurality of compartments comprising a first compartment and a second compartment, wherein:
the first compartment comprises a first target nucleic acid and a first barcoded primer comprising a first primer sequence for the first target nucleic acid and a first thermal barcode sequence; and
the second compartment comprises a second target nucleic acid and a barcoded primer comprising a second primer sequence for the second target nucleic acid and a second thermal barcode sequence;
wherein a peak melting temperature of the first thermal barcode sequence differs from a peak melting temperature of the second thermal barcode sequence by at least 0.1° C.

39. The article of claim 38, wherein the plurality of compartments comprises a plurality of droplets.

40. The article of claim 38, wherein the plurality of compartments comprises a plurality of microwells.

41. An article, comprising:
a plurality of compartments comprising a first compartment and a second compartment, wherein:
the first compartment comprises a first target nucleic acid and a first barcoded primer comprising a first primer sequence for the first target nucleic acid and a first thermal barcode sequence; and
the second compartment comprises a second target nucleic acid and a second barcoded primer comprising a second primer sequence for the second target nucleic acid and a second thermal barcode sequence;
wherein the first thermal barcode sequence has a first ratio of total nearest-neighbor binding enthalpy to total nearest-neighbor binding entropy and the second thermal barcode sequence has a second ratio of total nearest-neighbor binding enthalpy to total nearest-neighbor binding entropy, wherein the first ratio differs from the second ratio by at least 1% of the second ratio.

42. The article of claim 41, wherein the plurality of compartments comprises a plurality of droplets.

43. The article of claim 41, wherein the plurality of compartments comprises a plurality of microwells.

44. A method, comprising:
selectively barcoding a target nucleic acid using a thermal barcode to form a barcoded target nucleic acid, and
amplifying the barcoded target nucleic acid,
wherein the thermal barcode exhibits a first peak melting point and the target nucleic acid exhibits a second peak melting point, the first peak melting point and the second peak melting point differing by at least 0.1° C.

45. The method of claim 44, further comprising determining the barcoded target nucleic acid by determining the first peak melting point and the second peak melting point.

46. A method, comprising:
selectively barcoding a target nucleic acid using a thermal barcode to form a barcoded target nucleic acid, and
amplifying the barcoded target nucleic acid,
wherein the thermal barcode sequence has a first ratio of total nearest-neighbor binding enthalpy to total nearest-neighbor binding entropy and the target nucleic acid has a second ratio of total nearest-neighbor binding enthalpy to total nearest-neighbor binding entropy, wherein the first ratio differs from the second ratio by at least 1% of the second ratio.

47. A method, comprising:
selectively barcoding a target nucleic acid using a thermal barcode to form a barcoded target nucleic acid, and
amplifying the barcoded target nucleic acid,
wherein the thermal barcode sequence comprises less than or equal to 500 nucleotides and wherein at least 55% of the nucleotides of the thermal barcode sequence are A or T or wherein at least 45% of the nucleotides of the thermal barcode are G or C, and
wherein the thermal barcode sequence exhibits a peak melting temperature first peak melting point and the target nucleic acid exhibits a second peak melting point, the first peak melting point and the second peak melting point differing by at least 0.1° C.

48. A method as in any one of claims 44-47, wherein the barcoding is performed by ligation and/or reverse transcription of the target nucleic acid to form a complimentary, barcoded target nucleic acid.

49. A method as in any one of claims 44-47, wherein barcoding is performed using CRISPR.

50. A method as in any one of claims 44-49, wherein determining the barcoded target nucleic acid comprises determining the presence or the absence of the target nucleic acid within a sample.

51. A method as in any one of claims 44-50, wherein determining the barcoded target nucleic acid comprises using one or more auxiliary barcodes configured to bind to at least a portion of the thermal barcode sequence or at least a portion of the target nucleic acid.

52. A method as in any one of claims 44-51, wherein the presence of the barcoded target nucleic acid is indicative of a disease or condition.

53. A method as in any one of claims 44-52, wherein the absence of the barcoded target nucleic acid is indicative of a disease or condition.

54. A method as in any one of claims 44-53, wherein determining the barcoded target nucleic acid comprises determining a subject from which the target nucleic acid originated.

55. A method as in any one of claims 44-54, wherein the target nucleic acid is a first target nucleic acid of a plurality of target nucleic acids.

56. The method of any one of claims 44-55, wherein a compartment comprises the nucleic acid.

57. The method of any one of claims 44-56, wherein a droplet comprises the nucleic acid.

58. The method of any one of claims 44-57, wherein a microwell comprises the nucleic acid.

59. A method as in any one of claims 44-58, wherein the method comprises ligating a plurality of target nucleic acids using a plurality of ligation tags comprising a plurality of thermal barcode sequences to produce a plurality of barcoded target nucleic acids, and determining at least some of the plurality of barcoded target nucleic acids at least in part by measuring a peak melting temperature or a temperature-dependent melting profile of the thermal barcode sequences.

60. A method as in any one of claims 44-59, wherein the method further comprises forming a first plurality of droplets, at least 90% of which contain either only one target nucleic acid or no target nucleic acid, and at least 90% of which contain at least one thermal barcode.

61. A method as in any one of claims 44-60, wherein the method further comprises breaking the first plurality of droplets to mix the amplified nucleic acids.

62. A method as in any one of claims 44-61, wherein the method further comprises forming a second plurality of droplets, at least 90% of which contains either one of the amplified nucleic acids or no amplified nucleic acid, and at least 90% of which contain at least one thermal barcode.

63. A method as in any one of claims 44-62, wherein determining the nucleic acid further comprises exposing detecting the target nucleic acid and/or the barcoded target nucleic acid using an optical signaling entity.

64. A method as in any one of claims 44-63, wherein the optical signaling entity comprises a fluorophore.

65. A method as in any one of claims 44-64, wherein the optical signaling entity comprises a fluorescent quencher.

66. A method as in any one of claims 44-65, wherein the first thermal barcode sequence has a peak melting temperature that differs by at least 1° C. from a peak melting temperature of all other thermal barcode sequences of the plurality of thermal barcode sequences.

67. A method as in any one of claims 44-66, wherein the plurality of thermal barcode sequences each have distinct temperature-dependent melting profiles.

68. A method as in any one of claims 44-67, wherein the temperature-dependent melting profile of the thermal barcode sequence differs from a temperature-dependent melting profile of the target nucleic acid.

69. A method as in any one of claims 44-68, wherein the thermal barcode sequence melts within a first temperature range and the target nucleic acid melts within a second temperature range different from the first temperature range.

70. A method as in any one of claims 44-69, wherein the first temperature range is separated from the second temperature range by greater than or equal to 2° C.

71. A method as in any one of claims 44-70, wherein the first temperature range is totally below the second temperature range.

72. A method as in any one of claims 44-71, wherein the first temperature range is totally above the second temperature range.
